(19) United States
(12) Patent Application Publication
(10) Pub. No.: US 2022/0202900 A1
(43) Pub. Date: Jun. 30, 2022

(54) COMPOSITIONS AND METHODS FOR CRISPR/CAS9 KNOCK-OUT OF CD33 IN HUMAN HEMATOPOIETIC STEM / PROGENITOR CELLS FOR ALLOGENIC TRANSPLANTATION IN PATIENTS WITH RELAPSED - REFRACTORY ACUTE MYELOID LEUKEMIA

(71) Applicant: THE TRUSTEES OF THE UNIVERSITY OF PENNSYLVANIA, Philadelphia, PA (US)

(72) Inventors: Saar GILL, Philadelphia, PA (US); Miriam Y. KIM, Philadelphia, PA (US)

(21) Appl. No.: 17/432,880

(22) PCT Filed: Feb. 21, 2020
(86) PCT No.: PCT/US2020/019370

§ 371 Date: Aug. 20, 2021

Related U.S. Application Data
(60) Provisional application No. 62/809,448, filed on Feb. 22, 2019.

(51) Int. Cl. A61K 38/17; C12N 15/11; C12N 15/90; C12N 9/22; A61K 35/17; A61K 35/28; A61K 31/7105; A61P 35/00

(52) U.S. Cl. CPC A61K 38/1774; C12N 15/11; C12N 15/907; C12N 9/22; C12N 2800/80; A61K 35/28; A61K 31/7105; A61P 35/00; C12N 2310/20; A61K 35/17

(57) ABSTRACT

The present invention includes compositions and methods utilizing chemically modified CD33-targeting guide RNAs.

### CROSS-REFERENCE TO RELATED APPLICATION

The present application is entitled to priority under 35 U.S.C. § 119(e) to U.S. Provisional Patent Application No. 62/809,448, filed Feb. 22, 2019, which is hereby incorporated by reference in its entirety herein.

### STATEMENT REGARDING FEDERALLY SPONSORED RESEARCH OR DEVELOPMENT

This invention was made with government support under CA214278-02 awarded by the National Institutes of Health. The government has certain rights in the invention.

### BACKGROUND OF THE INVENTION

Recent advances in cancer immunotherapy with chimeric antigen receptor (CAR) T cells have enabled eradication of cells expressing a specific surface antigen. While this approach has been successful in targeting CD19 in B cell neoplasms, it does not discriminate between normal and malignant B cells, and thus, the feasibility of this therapy rests on the tolerability of prolonged B cell aplasia. However, most malignancies do not have an expendable normal tissue counterpart, and whether the success of CAR-T cells can be extrapolated beyond B cell neoplasms will depend on the ability to develop strategies to mitigate toxicity to normal cells.

The majority of acute myeloid leukemia (AML) patients relapse despite intensive therapy. AML cell surface antigens are shared with normal myeloid progenitors; therefore, targeting AML also generates toxicity to the myeloid system. While strategies to produce transient CAR T cells are being explored in clinical trials to avoid long-term myeloablation while targeting AML, this negates a fundamental strength of this therapy—namely, its long-term anti-tumor immune surveillance. Therefore, in the absence of a truly AML-specific antigen, a need exists for novel approaches to definitively target AML while sparing normal hematopoiesis. The present invention addresses this need.

### SUMMARY OF THE INVENTION

One aspect of the invention includes a chemically modified CD33-targeting guide RNA comprising a crRNA portion comprising (i) a guide sequence capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides.

Another aspect of the invention includes a chemically modified CD33-targeting guide RNA comprising a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence, wherein the crRNA portion and/or the tracrRNA portion comprises one or more modified nucleotides.

Yet another aspect of the invention includes a CD33-targeting guide RNA comprising a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence.

Another aspect of the invention includes a chemically modified CD33-targeting guide RNA comprising: a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence, wherein the crRNA portion and/or the tracrRNA portion comprise(s) one or more modified nucleotides.

Another aspect of the invention includes a CD33-targeting guide RNA comprising: a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence.

Still another aspect of the invention includes a method for generating a population of modified hematopoietic stem and progenitor cells (HSPCs). The method comprises introducing into the HSPCs a CD33-targeting guide RNA of the present invention, wherein the guide RNA produces an insertion and/or deletion capable of downregulating gene expression of CD33.

In another aspect, the invention includes a method for generating a population of modified HSPCs, comprising introducing into the HSPCs a CD33-targeting guide RNA comprising: a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence.

In yet another aspect, the invention includes a method for generating a population of modified HSPCs. The method comprises introducing into the HSPCs a chemically modified CD33-targeting guide RNA comprising: a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides, and the chemically modified guide RNA produces an insertion and/or deletion capable of downregulating gene expression of CD33.

In still another aspect, the invention includes a method for generating a population of modified HSPCs, comprising introducing into the HSPCs a CD33-targeting guide RNA comprising: a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. Also introduced into the HSPCs is a single-stranded oligonucleotide donor (ssODN) sequence, comprising the nucleotide sequence GCAGGAGTCAGTGACGGTACAAGGAGGGTTTG TGCGTCC (SEQ ID NO:3).

Another aspect of the invention includes a method for generating a population of modified HSPCs comprising introducing into the HSPCs a chemically modified CD33-targeting guide RNA. The guide RNA comprises a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides and the chemically modified guide RNA produces an insertion and/or deletion capable of downregulating gene expression of CD33. Also introduced into the HSPCs is a single-stranded oligonucleotide donor (ssODN) sequence comprising the nucleotide sequence

(SEQ ID NO: 3)
GCAGGAGTCAGTGACGGTACAAGGAGGGTTTGTGCGTCC.

Yet another aspect of the invention includes a method for generating a population of modified HSPCs, comprising introducing into the HSPCs a chemically modified CD33-targeting guide RNA comprising: a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides, and the chemically modified guide RNA produces an insertion and/or deletion capable of downregulating gene expression of CD33.

Another aspect of the invention includes a method for generating a population of modified hematopoietic stem and progenitor cells (HSPCs) comprising introducing into the HSPCs a CD33-targeting guide RNA comprising: a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence.

Another aspect of the invention includes a method for generating a population of modified hematopoietic stem and progenitor cells (HSPCs) comprising introducing into the HSPCs a chemically modified CD33-targeting guide RNA comprising: a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides, and the chemically modified guide RNA produces an insertion and/or deletion capable of downregulating gene expression of CD33.

Yet another aspect of the invention includes a method for generating a population of modified hematopoietic stem and progenitor cells (HSPCs) comprising: introducing into the HSPCs a CD33-targeting guide RNA comprising: a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence; and introducing into the HSPCs a single-stranded oligonucleotide donor (ssODN) sequence, wherein the ssODN sequence comprises the nucleotide sequence GCAGGAGTCAGTGACGGTACAAGGAGGGTTTGTGCGTCC (SEQ ID NO:3).

Still another aspect of the invention includes a method for generating a population of modified hematopoietic stem and progenitor cells (HSPCs). The method comprises introducing into the HSPCs a chemically modified CD33-targeting guide RNA comprising: a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides. The chemically modified guide RNA produces an insertion and/or deletion capable of downregulating gene expression of CD33. Also introduced into the HSPCs is a single-stranded oligonucleotide donor (ssODN) sequence. The ssODN sequence comprises the nucleotide sequence GCAGGAGTCAGTGACGGTACAAGGAGGGTTTGTGCGTCC (SEQ ID NO:3).

Another aspect of the invention includes a method for generating a population of modified hematopoietic stem and progenitor cells (HSPCs) comprising introducing into the HSPCs a chemically modified CD33-targeting guide RNA comprising: a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprise(s) one or more modified nucleotides, and the chemically modified guide RNA produces an insertion and/or deletion capable of downregulating gene expression of CD33.

Still another aspect of the invention includes a population of modified hematopoietic stem and progenitor cells (HSPCs) generated by any one of the methods of the present invention.

In one aspect, the invention includes a population of modified HSPCs, wherein the HSPCs comprise an insertion and/or deletion in an endogenous gene locus encoding for CD33. The insertion and/or deletion is mediated by a CD33-targeting guide RNA comprising: a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence.

In another aspect, the invention includes a population of modified HSPCs, wherein the HSPCs comprise an insertion and/or deletion in an endogenous gene locus encoding for CD33. The insertion and/or deletion is mediated by a CD33-targeting guide RNA and a single-stranded oligonucleotide donor (ssODN) sequence. The guide RNA comprises a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. The ssODN sequence comprises the nucleotide sequence GCAGGAGTCAGTGACGGTACAAGGAGGGTTTGTG CGTCC (SEQ ID NO:3).

In another aspect, the invention includes a population of modified HSPCs, wherein the HSPCs comprise an insertion and/or deletion in an endogenous gene locus encoding for CD33. The insertion and/or deletion is mediated by a chemically modified CD33-targeting guide RNA comprising: a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides, and the insertion and/or deletion is capable of downregulating gene expression of CD33.

In yet another aspect, the invention includes a population of modified HSPCs. The HSPCs comprise an insertion and/or deletion in an endogenous gene locus encoding for CD33. The insertion and/or deletion is mediated by: a chemically modified CD33-targeting guide RNA and a single-stranded oligonucleotide donor (ssODN) sequence. The guide RNA comprises a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides, and the insertion and/or deletion is capable of downregulating gene expression of CD33. The ssODN sequence comprises the nucleotide sequence GCAGGAGTCAGTGACGGTACAAGGAGGGTTTGTGCGTCC (SEQ ID NO:3).

Another aspect of the invention includes a population of modified hematopoietic stem and progenitor cells (HSPCs), wherein the HSPCs comprise an insertion and/or deletion in an endogenous gene locus encoding for CD33. The insertion and/or deletion is mediated by a CD33-targeting guide RNA comprising: a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence.

Yet another aspect of the invention includes a population of modified hematopoietic stem and progenitor cells (HSPCs), wherein the HSPCs comprise an insertion and/or deletion in an endogenous gene locus encoding for CD33. The insertion and/or deletion is mediated by a CD33-targeting guide RNA comprising: a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence; and a single-stranded oligonucleotide donor (ssODN) sequence. The ssODN sequence comprises the nucleotide sequence GCAGGAGTCAGTGACGGTACAAGGAGGG TTTGTGCGTCC (SEQ ID NO:3).

Still another aspect of the invention includes a population of modified hematopoietic stem and progenitor cells (HSPCs), wherein the HSPCs comprise an insertion and/or deletion in an endogenous gene locus encoding for CD33. The insertion and/or deletion is mediated by a chemically modified CD33-targeting guide RNA comprising: a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides, and wherein the insertion and/or deletion is capable of downregulating gene expression of CD33.

Another aspect of the invention includes a population of modified hematopoietic stem and progenitor cells (HSPCs), wherein the HSPCs comprise an insertion and/or deletion in an endogenous gene locus encoding for CD33. The insertion and/or deletion is mediated by a chemically modified CD33-targeting guide RNA comprising: a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence, wherein the crRNA portion and/or the tracrRNA portion comprise(s) one or more modified nucleotides, and wherein the insertion and/or deletion is capable of downregulating gene expression of CD33; and a single-stranded oligonucleotide donor (ssODN) sequence. The ssODN sequence comprises the nucleotide sequence

(SEQ ID NO: 3)
GCAGGAGTCAGTGACGGTACAAGGAGGGTTTGTGCGTCC.

In still another aspect, the invention includes a method of treating a cancer in a subject in need thereof. The method comprises administering to the subject a CD33-targeted cell therapy comprising a CD33 CAR-T cell and a population of modified HSPCs that are resistant to the CD33-targeted therapy disclosed herein.

In one aspect, the invention includes a method of treating a cancer in a subject in need thereof, comprising administering to the subject a CD33-targeted cell therapy comprising a CD33 CAR-T cell and a population of modified HSPCs that are resistant to the CD33-targeted therapy. The HSPCs comprise an insertion and/or deletion in an endogenous gene locus encoding for CD33. The insertion and/or deletion is mediated by a CD33-targeting guide RNA comprising: a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. The insertion and/or deletion is capable of downregulating gene expression of CD33. Thereby the method treats cancer in the subject.

In another aspect, the invention includes a method of treating a cancer in a subject in need thereof, comprising administering to the subject a CD33-targeted cell therapy comprising a CD33 CAR-T cell and a population of modified HSPCs that are resistant to the CD33-targeted therapy. The HSPCs comprise an insertion and/or deletion in an endogenous gene locus encoding for CD33. The insertion and/or deletion is mediated by a CD33-targeting guide RNA and a single-stranded oligonucleotide donor (ssODN) sequence. The guide RNA comprises a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. The ssODN sequence comprises the nucleotide sequence GCAGGAGTCAGTGACGGTACAAGGAGGGTTTGT GCGTCC (SEQ ID NO:3). The insertion and/or deletion is capable of downregulating gene expression of CD33, and the method thereby treats cancer in the subject.

In another aspect, the invention includes a method of treating a cancer in a subject in need thereof, comprising administering to the subject a CD33-targeted cell therapy comprising a CD33 CAR-T cell and a population of modified HSPCs that are resistant to the CD33-targeted therapy. The HSPCs comprise an insertion and/or deletion in an endogenous gene locus encoding for CD33, wherein the insertion and/or deletion is mediated by a chemically modified CD33-targeting guide RNA. The guide RNA comprises a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides. The insertion and/or deletion is capable of downregulating gene expression of CD33, and the method thereby treats cancer in the subject.

In another aspect, the invention includes a method of treating a cancer in a subject in need thereof, comprising administering to the subject a CD33-targeted cell therapy comprising a CD33 CAR-T cell; and a population of modified HSPCs that are resistant to the CD33-targeted therapy The HSPCs comprise an insertion and/or deletion in an endogenous gene locus encoding for CD33. The insertion and/or deletion is mediated by a chemically modified CD33-targeting guide RNA and a single-stranded oligonucleotide donor (ssODN) sequenc. The guide RNA comprises a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides. The ssODN sequence comprises the nucleotide sequence GCAGGAGTCAGTGACGGTACAAGGAGGG TTTGTGCGTCC (SEQ ID NO:3). The insertion and/or deletion is capable of downregulating gene expression of CD33, and the cancer is thereby treated in the subject.

In yet another aspect, the invention includes a method of treating a cancer in a subject in need thereof. The method comprises administering to the subject a CD33-targeted cell therapy comprising a CD33 CAR-T cell a population of modified hematopoietic stem and progenitor cells (HSPCs) that are resistant to the CD33-targeted therapy. The HSPCs comprise an insertion and/or deletion in an endogenous gene locus encoding for CD33. The insertion and/or deletion is mediated by a CD33-targeting guide RNA comprising: a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. The insertion and/or deletion is capable of downregulating gene expression of CD33, thereby treating cancer in the subject.

In still another aspect, the invention includes a method of treating a cancer in a subject in need thereof. The method comprises administering to the subject a CD33-targeted cell therapy comprising a CD33 CAR-T cell and a population of modified hematopoietic stem and progenitor cells (HSPCs) that are resistant to the CD33-targeted therapy. The HSPCs comprise an insertion and/or deletion in an endogenous gene locus encoding for CD33. The insertion and/or deletion is mediated by: a CD33-targeting guide RNA comprising: a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence; and a single-stranded oligonucleotide donor (ssODN) sequence. The ssODN sequence comprises the nucleotide sequence GCAGGAGTCAGTGACGGTACAAGGAGGGTTTGTGCGTCC (SEQ ID NO:3). The insertion and/or deletion is capable of downregulating gene expression of CD33, thereby treating cancer in the subject.

Another aspect of the invention includes a method of treating a cancer in a subject in need thereof. The method comprises administering to the subject a CD33-targeted cell therapy comprising a CD33 CAR-T cell and administering to the subject a population of modified hematopoietic stem and progenitor cells (HSPCs) that are resistant to the CD33-targeted therapy. The HSPCs comprise an insertion and/or deletion in an endogenous gene locus encoding for CD33. The insertion and/or deletion is mediated by a chemically modified CD33-targeting guide RNA comprising: a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides, and the insertion and/or deletion is capable of downregulating gene expression of CD33, thereby treating cancer in the subject.

Yet another aspect of the invention includes a method of treating a cancer in a subject in need thereof. The method comprises administering to the subject a CD33-targeted cell therapy comprising a CD33 CAR-T cell and administering to the subject a population of modified hematopoietic stem and progenitor cells (HSPCs) that are resistant to the CD33-targeted therapy. The HSPCs comprise an insertion and/or deletion in an endogenous gene locus encoding for CD33. The insertion and/or deletion is mediated by a chemically modified CD33-targeting guide RNA comprising: a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. The crRNA portion and/or the tracrRNA portion comprises one or more modified nucleotides; and a single-stranded oligonucleotide donor (ssODN) sequence. The ssODN sequence comprises the nucleotide sequence GCAGGAGTCAGTGACGGTACAAGGAGGGTTTGTGCGTCC (SEQ ID NO:3). The insertion and/or deletion is capable of downregulating gene expression of CD33, thereby treating cancer in the subject.

Another aspect of the invention includes a gene editing system comprising: a) a chemically modified CD33-targeting guide RNA comprising a crRNA portion and a tracrRNA portion and b) an RNA-guided nuclease. The crRNA portion comprises a guide sequence capable of hybridizing to a target endogenous gene locus encoding for CD33, and a repeat sequence. The tracrRNA portion comprises an anti-repeat sequence that is complementary to the repeat sequence. The crRNA portion and/or the tracrRNA portion comprise(s) one or more modified nucleotides.

In various embodiments of the above aspects or any other aspect of the invention delineated herein, the crRNA portion of the guide RNA comprises one or more modified nucleotides. In certain embodiments, the tracrRNA portion of the guide RNA comprises one or more modified nucleotides. In certain embodiments, the crRNA portion and the tracrRNA portion comprises one or more modified nucleotides.

In certain embodiments, the one or more modified nucleotides comprise a modification of a ribose group, a phosphate group, or a combination thereof. In certain embodiments, the modification of the ribose group is selected from 2′-O-methyl, 2′-fluoro, 2′-deoxy, 2′-O-(2-methoxyethyl) (MOE), 2′-NH₂, or a bicyclic nucleotide such as locked nucleic acid (LNA), 2′-(S)-constrained ethyl (S-cEt), constrained MOE, or 2′-0,4′-C-aminomethylene bridged nucleic acid (2′,4′-BNA^{NC}). In certain embodiments, the modification of the phosphate group comprises a phosphorothioate, phosphonoacetate (PACE), thiophosphonoacetate (thioPACE), amide, triazole, phosphonate, or phosphotriester modification.

In certain embodiments, the nucleotides at positions 1-3 from the 5′ end of the crRNA portion comprise 2′-O-methyl modifications. In certain embodiments, nucleotides at positions 2-4 from the 3′ end of the tracrRNA portion comprise 2′-O-methyl modifications. In certain embodiments, nucleotides at positions 1-4 from the 5′ end of the crRNA portion comprise phosphorothioate modifications. In certain embodiments, nucleotides at positions 1-4 from the 3′ end of the tracrRNA portion comprise phosphorothioate modifications.

In certain embodiments, the guide sequence comprises the nucleic acid sequence GUCAGUGACGGUACAGGA (SEQ ID NO:1). In certain embodiments, the guide sequence consists of the nucleic acid sequence GUCAGUGACGGUACAGGA (SEQ ID NO:1).

In certain embodiments, the guide sequence comprises the nucleic acid sequence mG*mU*mC*AGUGACGGUACAGGA (SEQ ID NO: 2), wherein m indicates a 2′-O-methyl modification, and wherein * indicates a phosphorothioate modification. In certain embodiments, the guide sequence consists of the nucleic acid sequence mG*mU*mC*AGUGACGGUACAGG A (SEQ ID NO: 2), wherein m indicates a 2′-O-methyl modification, and wherein * indicates a phosphorothioate modification.

In certain embodiments, the guide sequence comprises the nucleic acid sequence GAGUCAGUGACGGUACAGGA (SEQ ID NO:16). In certain embodiments, the guide sequence consists of the nucleic acid sequence GAGUCAGUGACGGUACAGGA (SEQ ID NO:16).

In certain embodiments, the guide sequence comprises the nucleic acid sequence mG*mA*mG*UCAGUGACGGUACAGGA (SEQ ID NO: 17), wherein m indicates a 2′-O-methyl modification, and wherein * indicates a phosphorothioate modification. In certain embodiments, the guide sequence consists of the nucleic acid sequence mG*mA*mG*UCAGUGACGGUACAGGA (SEQ ID NO: 17), wherein m indicates a 2′-O-methyl modification, and wherein * indicates a phosphorothioate modification.

In certain embodiments, the guide RNA is in a complex with a Cas9 nuclease.

In certain embodiments, the Cas9 nuclease is an S. pyogenes Cas9 nuclease or an S. aureus Cas9 nuclease. In certain embodiments, the Cas9 nuclease is a variant S. pyogenes Cas9 nuclease. In certain embodiments, the variant Cas9 nuclease comprises reduced off-target activity, and maintained or increased on-target activity relative to a wild-type Cas9. In certain embodiments, the variant Cas9 nuclease is a SpyFi™ Cas9 nuclease.

In certain embodiments, the HSPCs are resistant to a CD33-targeted cell therapy. In certain embodiments, the CD33-targeted cell therapy comprises a CD33 CAR-T therapy.

In certain embodiments, the HSPCs are autologous cells obtained from a source selected from the group consisting of peripheral blood mononuclear cells, cord blood cells, bone marrow, lymph node, and spleen. In certain embodiments, the HSPCs are CD34+ HSPCs.

In certain embodiments, the method of the invention further comprises introducing into the HSPCs a homology-directed repair (HDR) template comprising a single-stranded oligonucleotide donor (ssODN) sequence.

In certain embodiments, the ssODN sequence comprises (i) the nucleotide sequence GCAGGAGTCAGTGACGGTACAGGAGGGTTTGTGCGTCC (SEQ ID NO:18) with an extra adenine inserted, or (ii) the nucleotide sequence

(SEQ ID NO: 3)
GCAGGAGTCAGTGACGGTACAAGGAGGGTTTGTGCGTCC.

In certain embodiments, the ssODN sequence consists of (i) the nucleotide sequence GCAGGAGTCAGTGACGGTACAGGAGGGTTTGTGCGTCC (SEQ ID NO: 18) with an extra adenine inserted, or (ii) the nucleotide sequence GCAGGAGTCAGTGACGGTACAAGGAGG GTTTGTGCGTCC (SEQ ID NO:3).

In certain embodiments, the ssODN comprises the nucleotide sequence

(SEQ ID NO: 4)
GGTTGTCGGGCTGGGCCGAGCTGACCCTCGTTTCCCCACAGGGGCCCTGG
CTATGGATCCAAATTTCTGGCTGCAAGTGCAGGAGTCAGTGACGGTACAA
GGAGGGTTTGTGCGTCCTCGTGCCCTGCACTTTCTTCCATCCCATACCCT
ACTACGACAAGAACTCCCCAGTTCATGGTTACTGGTTCCGGGAAGGAGC.

In certain embodiments, the ssODN consists of the nucleotide sequence

(SEQ ID NO: 4)
GGTTGTCGGGCTGGGCCGAGCTGACCCTCGTTTCCCCACAGGGGCCCTGG
CTATGGATCCAAATTTCTGGCTGCAAGTGCAGGAGTCAGTGACGGTACAA
GGAGGGTTTGTGCGTCCTCGTGCCCTGCACTTTCTTCCATCCCATACCCT
ACTACGACAAGAACTCCCCAGTTCATGGTTACTGGTTCCGGGAAGGAGC.

In certain embodiments, the population of modified HSPCs comprise at least about 20%, 25%, 30%, 35%, 40%, 45%, 50%, 55%, 60%, 65%, 70%, 75%, 80%, 85%, 90%, 95% modified HSPCs comprising the insertion and/or deletion capable of downregulating gene expression of CD33.

In certain embodiments, the HSPCs are administered to the subject prior to the CD33-targeted therapy. In certain embodiments, the cancer is acute myeloid leukemia (AML).

### BRIEF DESCRIPTION OF THE DRAWINGS

The following detailed description of specific embodiments of the invention will be better understood when read in conjunction with the appended drawings. For the purpose of illustrating the invention, exemplary embodiments are shown in the drawings. It should be understood, however, that the invention is not limited to the precise arrangements and instrumentalities of the embodiments shown in the drawings.

FIGS. 1A-1L illustrate the in vitro generation of CD33 knockout human HSPCs. FIG. 1A displays representative flow plots showing the effects of full-length (20 bp) and truncated (17-18 bp) guide RNAs (gRNAs) on CD33 expression. gRNAs were tested for editing efficiency in a human AML cell line, Molm14, after electroporation with Cas9. FIG. 1B displays results from human CD34+ cells that were electroporated with Cas9 protein and truncated CD33-targeting gRNA4 from FIG. 1A. Surface expression of CD33 was assessed following 7 days of in vitro culture by flow cytometry (left) and DNA mutations were quantified by SURVEYOR assay (right). FIG. 1C shows the Sanger sequencing of individual alleles and reveals a high proportion of mutations containing a single A insertion (top); a representative chromatogram is shown (bottom). FIG. 1D is a schematic of a single-stranded oligo deoxynucleotide (ssODN) designed to contain 99-bp homology arms around the gRNA cut site and the A insertion mutation (top). CD34+ cells were electroporated with Cas9 protein/gRNA and different concentrations of the ssODN, after which CD33 expression was assessed by flow cytometry (bottom left) following 7 days of culture. Addition of the ssODN increases the frequency of CD33 mutations in three different donors in three independent experiments (bottom right; paired t test). FIG. 1E shows an experimental scheme for electroporating human CD34+ cells with either Cas9 complexed with EMX1-gRNA (Control) or Cas9/CD33-gRNA4 and the ssODN (CD33 KO) and cultured for 7 days. FIG. 1F shows that cultured CD33 KO CD34+ cells have decreased levels of surface CD33 expression by flow cytometry compared to controls (n=21, 18 different donors, 14 independent experiments). Representative flow cytometry plots are shown on the right (unpaired t test). FIG. 1G shows the growth kinetics of control and CD33 KO CD34+ cells maintained in StemSpan SFEM with SCF (100 ng/mL), Flt3L (100 ng/mL), TPO (50 ng/mL), and IL-6 (50 ng/uL) (n=3, 3 donors, 2 independent experiments; unpaired t test). FIG. 1H shows colony formation of CD34+ cells plated in Methocult H4435 1 day after electroporation (n=3, 3 donors, 2 independent experiments; unpaired t test). FIG. 1I shows the frequency of expression of myeloid markers CD33, CD14, and CD11b in methocult cultures (n=3, 3 donors, 2 independent experiments; unpaired t test). FIG. 1J reveals that in vitro-differentiated control and CD33 KO HSPCs show normal myeloid morphology by Wright-Giemsa staining with decreased CD33 expression by immunocytochemistry in the CD33 KO cells only. FIG. 1K illustrates comparison of levels of CD33 protein loss by flow cytometry and CD33 gene mutations by TIDE analysis show a high level of correlation (n=18). Percent of CD33 (protein) was calculated as follows: {1−(% CD33+ in KO)/(% CD33+ in control)}*100. FIG. 1L illustrates targeted amplicon sequencing of the gRNA site from CD33 KO cell DNA confirms high levels of total mutations, with the majority consisting of A insertions. All data are represented as means±SD. ns, not significant (p>0.05), *p<0.05, ****p<0.0001.

FIGS. 2A-2F illustrate the finding that CD33 KO human HSPCs show sustained loss of CD33 in vivo without impairment of growth and differentiation. FIG. 2A shows an experimental schematic where NSG mice were injected with 1-5×10⁵ control or CD33 KO HSPCs, and human CD45+ hematopoiesis was analyzed over time. FIG. 2B illustrates a decreased fraction of CD33+ monocytes in CD33 KO HSPC-engrafted mice compared to controls following longitudinal monitoring of human CD14+ monocytes ****p<0.0001 (unpaired t test). FIG. 2C shows CD33 KO HSPC-engrafted mice have significantly decreased numbers of CD33+ cells in the peripheral blood at 12 weeks post-transplant, while total numbers of cells expressing CD45, CD14, and CD11b are similar to controls (n=57 mice, 4 independent experiments, 5 donors). ns, not significant (p>0.05); ***p<0.001 (unpaired t test). FIG. 2D illustrates that bone marrow harvested from mice after 12-16 weeks of HSPC engraftment shows equivalent levels of human CD45+ cell engraftment, with differentiation into both lymphoid (B cells: CD19+, T cells: CD3+) and myeloid lineages, while myeloid cells have markedly decreased levels of CD33. Human hematopoietic stem (CD34+38) and progenitor (CD34+38+) cell levels are comparable between control and CD33 KO HSPC-engrafted mice. ****p<0.0001; ns, not significant (p>0.05) (unpaired t test) (n=20 mice, 3 independent experiments, 3 donors). FIG. 2E illustrates results from an experiment wherein marrow was harvested from mice engrafted with control or CD33 KO HSPCs after 16 weeks (primary), and human CD45+ cells were purified and injected into secondary recipients (secondary; each donor transplanted into a single recipient) and monitored for an additional 12 weeks. Both groups of mice show sustained human hematopoiesis after long-term engraftment (left), and the CD33 KO HSPC-engrafted group had persistent loss of CD33 expression (right; n=12 mice, 3 independent experiments, 4 donors). ****p<0.0001; ns, not significant (p>0.05) (unpaired t test). FIG. 2F shows results of PCR performed on marrow from secondary recipients and confirms continued presence of mutations in the CD33 gene by SURVEYOR assay (left). Mutation levels as quantified by TIDE are similar between the initial infusion product (input) and the bone marrow 28 weeks after transplantation (output; right). ns, not significant (p>0.05) (paired t test). All data are represented as mean±SD.

FIGS. 3A-3H illustrate the finding that that CD33 is not essential for human myeloid cell function. FIG. 3A illustrates bone marrow cells from NSG mice engrafted with control or CD33 KO human CD34+ cells exhibit normal human myeloid cell morphology, with decreased CD33 expression confirmed by immunocytochemistry. FIGS. 3B-3F show results from control and CD33 KO CD34+ cells differentiated in vitro with SCF, TPO, Flt3L, IL-3, IL-6, and GM-CSF for 7-14 days prior to functional assays. CD33+/cells within the CD33 KO group were gated separately by flow cytometry for analysis as compared to ungated control cells, which are all CD33+. FIG. 3B illustrates that in vitro-differentiated CD33 KO myeloid cells retain phagocytosis ability as measured by internalization of pH rodo green E. coli bioparticles (n=6 per group, one-way ANOVA). FIG. 3C displays the levels of reactive oxygen species (ROS) production after phorbol myristate acetate (PMA) stimulation. Levels are similar among the three groups of cells. ROS production was measured by fluorescence of CellROX Green reagent (n=5 per group, one-way ANOVA). FIG. 3D shows the expression of cytokines as measured by ICS. Expression is not significantly different in CD33 cells as compared to internal CD33+ or unedited controls, whether under basal conditions or after lipopolysaccharide (LPS) stimulation (n=5 per group, one-way ANOVA). FIG. 3E displays results of cells treated with the indicated stimuli (GM-CSF, G-CSF, IFNa, IFNg, IL-4, IL-6, LPS, PMA/ionomycin, or TPO) for 15 min followed by mass cytometry analysis using antibodies to 20 surface markers and 10 phosphoproteins for a comprehensive analysis of signaling pathways (n=3 per group). Control (ungated) and CD33+/gated cells within the CD33 KO cell group display identical signaling profiles within the myeloid progenitor population as defined by SPADE analysis. Representative plots of CD33 KO cells show that CD33 and residual CD33+ cell populations respond to the indicated stimuli to the same degree (gated on live CD64+HLADR+ events). FIG. 3F shows the gene expression profile of in vitro-differentiated control and CD33 KO CD34+ cells (with 70%-85% CD33 KO) that were analyzed by RNA-seq (n=5 per group). Log-scale scatterplot of mean gene expression values of control and CD33 KO samples show high correlation between the two groups (left), while volcano plot shows CD33 as the most significant differentially expressed gene (right). FIG. 3G illustrates that LPS injection induces similar levels of human inflammatory cytokine secretion in the serum of mice engrafted with control or CD33 KO HSPC (control: n=10, CD33 KO: n=12) (unpaired t test). FIG. 3H displays results from NSG mice engrafted with control or CD33 KO HSPCs that were injected with G-CSF and numbers of peripheral blood human myeloid cells were evaluated before and after treatment (n=9 per group, 2 donors, 2 independent experiments). Representative flow cytometry plot shows increase in both % CD14+CD33+ and % CD14+CD33 cells in the CD33 KO group after G-CSF injection (left), gating on single live human CD45+ events. Increased numbers of neutrophils (CD66b+) and monocytes (CD14+) were detected in the peripheral blood of both cohorts after G-CSF, and within the CD33 KO HSPC-engrafted mice, no difference in CD33+ or CD33− cell populations was detected (right) (one-way ANOVA). ns, not significant (p>0.05).

FIGS. 4A-4D illustrate the finding that CD33− human cells show no functional defects compared to CD33+ controls. FIGS. 4A-4D were generated from CD33 KO CD33+ cells that were differentiated in vitro with SCF, TPO, Flt3L, IL-3, IL-6 and GM-CSF for 7 days after which cells were sorted based on CD33 expression, and functional assays were performed as in FIGS. 3A-3D (n=5/group, 2 independent experiments). FIG. 4A displays data showing that CD33− cells CD33− cells can perform phagocytosis of E. coli bioparticles to the same degree as CD33+ cells. FIG. 4B shows that ROS production at basal and after PMA stimulation is almost identical between CD33− and CD33+ cells. FIG. 4C shows that intracellular cytokine production is similar between CD33+ and CD33− cells after LPS stimulation. FIG. 4D displays the results of cytokine/chemokine secretion measured in the supernatant before and after LPS stimulation for 24 hours. CD33-negative cells show the same degree of cytokine/chemokine secretion after LPS stimulation. All statistical tests were performed with unpaired Student's t test. ns=not significant.

FIGS. 5A-5C illustrate mass cytometry analysis of control and CD33 KO primary human CD34+ shows no difference in surface markers or intracellular signaling profiles. FIG. 5A is a SPADE diagram of a representative donor showing CD33 expression of in vitro differentiated control and CD33 KO primary CD34+ cells. SPADE clustering was performed on all 6 samples (3 donors, each with control and CD33 KO) simultaneously to generate a single tree structure for all samples, and all events from each sample were mapped to the common tree structure. CD33 expression is globally decreased across all groups in the CD33 KO cells, while differentiation profile (as depicted by node size) is similar to control cells. FIG. 5B displays heatmaps showing signaling profiles of myeloid cell clusters within control and CD33 KO cells in response to external stimuli. In vitro differentiated control and CD33 KO HSPC from three donors were treated with various stimuli (GM-CSF, G-CSF, IFNa, IFNg, IL-4, IL-6, LPS, PMA/ionomycin, and TPO), and mass cytometry was performed with antibodies to 20 surface markers and 10 phosphoproteins for comprehensive analysis of signaling. CD33+ and CD33− cells were analyzed separately within the CD33 KO cell population and compared to ungated controls. Groups are defined by the SPADE tree shown in (FIG. 5A). Scaled to arcsinh ratio versus basal of the same donor. One representative donor is shown. FIG. 5C displays additional flow cytometry plots of CD33 KO cells showing the indicated phosphoprotein response (on x axis) to stimuli depicted in box (gated on live CD64+HLA-DR+ events). CD33 is depicted on y axis. Shown are plots from one representative donor.

FIGS. 6A-6E illustrate the finding that human CD33 KO HSPC do not display any significant off-target events. FIG. 6A shows cytogenetic analysis of control and CD33 KO CD34+ cells cultured in vitro. No gross chromosomal abnormalities were observed. Shown is one representative karyogram of 6 different donors assessed. FIG. 6B shows an evaluation of chromosomal rearrangements between SIGLEC22P and CD33 from CRISPR/Cas9-mediated genetic modification. Left panel: Schematic of PCR primers (A, B, C, D) used to detect deletion or inversion of the DNA segment between the two genes. Guide RNA cut site is depicted by arrows within each gene. Right panel: PCR of DNA extracted from 6 control/CD33 KO HSPC human donors shows that deletion between the two genes (AD) occurs in all CD33 KO samples, while inversion (AC/BD) is not detected. FIG. 6C shows targeted deep sequencing of on-target and in silico predicted off-target sites in genomic DNA extracted from 5 human HSPC donors (A-E) after CD33 KO. FIG. 6D shows targeted deep sequencing of on-target and CIRCLE-seq predicted off-target sites in 3 human HSPC donors (E-G) after CD33 KO. 16 off-target sites were selected based on high CIRCLE-seq read count and/or low numbers of mismatch with the target site. FIG. 6E displays a visualization of all on-target and off-target sites detected by CIRCLE-seq in a primary human genomic DNA sample. CIRCLEseq was performed by in vitro digestion of genomic DNA with Cas9/CD33-gRNA; validation by targeted deep sequencing was performed on genomic DNA from cells that underwent the CD33 KO procedure then differentiated in vitro (donors E,G) as outlined in FIG. 1E, or differentiated in mice (donor F) as outlined in FIG. 2A.

FIGS. 7A-7I illustrate the finding that human myeloid cells lacking expression of CD33 are resistant to CD33-targeted therapy. FIG. 7A displays an experimental schema where NSG mice engrafted with control or CD33 KO HSPCs were treated with autologous anti-CD33 CAR T cells (CART33) followed by serial retro-orbital bleeding. Mice were euthanized, and bone marrow (BM) and spleen were analyzed 4 weeks after CART33 infusion. A subset of mice in each group did not receive CART33. (Control-no CART33: n=4, CD33 KO-no CART33: n=4, Control+CART33: n=15, CD33 KO+CART33: n=15, 2 independent experiments, 2 donors). FIG. 7B shows the numbers of CD14+ monocytes in the peripheral blood of CD33 KO HSPC-engrafted mice after CART33 treatment are similar to control HSPC mice without CART33 treatment (one-way ANOVA) (left). Representative flow cytometry plot (right) shows complete eradication of CD33+ cells in both groups after CART33 treatment, which leads to loss of CD14+ monocytes in the control HSPC-engrafted mice, while CD14+CD33 cells are still detected in CD33 KO HSPC-engrafted mice. Plots were gated on live singlet human CD45+CD19CD3 cells. FIG. 7C shows that CD14+ cells are present in significantly higher numbers in the spleen and bone marrow of CD33 KO HSPC-engrafted mice after CART33 treatment compared to controls (unpaired t test). FIG. 7D shows that bone marrow human stem cells (CD34+38) and progenitors (CD34+38+) are significantly higher after CART33 treatment in CD33 KO HSPC mice compared to controls (unpaired t test). Representative flow cytometry plot, gated on live singlet human CD45+ lineage-negative cells, shown on right. FIG. 7E displays an experimental schema where control or CD33 KO HSPC-engrafted mice were injected with Molm14-GFP/luciferase prior to treatment with CART33 (n=26 mice, 2 independent experiments, 3 donors). FIG. 7F illustrates that mice show the expected reduction in AML burden as measured by bioluminescent imaging (BLI) after CART33 treatment (unpaired t test). FIG. 7G shows that co-engraftment of AML does not impair the survival of CD33-negative myeloid cells in vivo after CART33 treatment in the peripheral blood (top; one-way ANOVA), spleen, and bone marrow (bottom; unpaired t test). FIG. 7H illustrates that peripheral blood CD33 levels decline after CART33 treatment in both cohorts of mice, resulting in undetectable levels of CD14+ cells in the control mice, while CD14+ cells persist in CD33 KO HSPC-engrafted mice (shown is one representative cohort of mice, single donor, n=4 per group). FIG. 7I shows that bone marrow human stem and progenitor cells continue to survive CART33-mediated attack, even in the setting of co-existing AML (unpaired t test). All data are represented as means±SD. ns, not significant (p>0.05); **p<0.01; ***p<0.001; ****p<0.0001.

FIG. 8A illustrates the finding that viability post electroporation was lower with in vitro transcribed (IVT) gRNA compared with synthetic modified gRNA (NTC=no template control, IDT=Integrated DNA Technologies gRNA, EO=pulse code EO-100, DZ=pulse code DZ-100). FIG. 8B illustrates the finding that no difference was seen in post thaw viability of electroporated cells between three different freeze media; CVPF and HUP are in-house products, and CS-10 is available commercially (CVPF IC+CD freeze media=7.5% DMSO, HUP freeze media 7.5% DMSO, CS-10=10% DMSO). FIGS. 8C-8D illustrate the finding that post electroporation viability was superior with culture in StemSpan (S) media compared with X-Vivo15 (X) across a range of conditions including different gRNA sources (no EP=no electroporation) and two different pulse codes EO-100 and DZ-100.

FIG. 9A shows a representative example of CD33 expression in myeloid cells cultured in vitro derived from CD34 cells 7 days after KO, with in vitro transcribed (IVT) gRNA consistently achieving higher KO efficiency, and IDT (Integrated DNA Technologies) synthetic gRNA out-performing Synthego (No EP=no electroporation, NTC=no template control). FIG. 9B illustrates the finding that KO efficiency was similar between StemLine (SL) and StemSpan (SP) media, however the addition of more CD34+ cells per ribonuclear protein (RNP) complex reduced KO efficiency in StemSpan media. FIG. 9C illustrates the finding that residual Cas9 levels were higher after electroporation with IDT gRNA compared with IVT gRNA, with no significant degradation of Cas9 at 48 hours compared with 24 hours post electroporation. FIG. 9D illustrates the finding that StemSpan media yielded a higher number of both erythroid and non-erythroid colonies after plating 1000 CD34+ cells after electroporation and freeze/thaw, with higher colony numbers seen when compared with StemLine media. Colony formation was higher in cells treated with IDT gRNA versus IVT.

FIG. 10 shows cytokine analysis of supernatant from CD34+ cell culture 24 hours after electroporation indicates less derangement with synthetic modified gRNA compared with IVT gRNA (IVT gRNA=in vitro transcribed guide RNA, IDT=Integrated DNA Technologies synthetic modified guide RNA, Synthego=Synthego synthetic modified guide RNA, NTC=no template control, No EP=no electroporation, EO=pulse code E0100, DZ=pulse code DZ100).

FIG. 11 illustrates titration of the double stranded oligodeoxynucleotide (dsODN) performed at the dilutions indicated, with no difference in cell viability noted. Note that RNP-cells are included to detect background double stranded break frequency not related to CRISPR/Cas9 activity.

FIG. 12 depicts a chemically-modified CD33-targeting guide RNA.

FIG. 13 illustrates two gRNAs used in the study. The difference in the gRNAs illustrated is two nucleotides at the 5′ end of the targeting sequence.

FIG. 14 illustrates on-target efficiency analyzed by flow cytometry. In descending order: 1.) TFv2 Cas9 with 20nt gRNA=SpyFi™ Cas9 with 20nt gRNA, 2.) TFv2 Cas9 with 18nt gRNA, 3.) SpyFi™ Cas9 with 18nt gRNA. TFv2=ThermoFisher® version 2; SpyFi=SpyFi™ Cas9 from Aldevron®. All gRNAs were chemically-modified.

FIG. 15 illustrates on-target efficiency, in descending order (most efficient to least efficient), analyzed by GUIDEseq: 1.) SpyFi™ Cas9 with 20nt gRNA, 2.) TFv2 Cas9 with 20nt gRNA, 3.) TFv2 Cas9 with 18nt gRNA, 4.) SpyFi™ Cas9 with 18nt gRNA; and off-target risk, in descending order (least risk to most risk), analyzed by GUIDEseq: 1.) SpyFi™ Cas9 with 20nt gRNA, 2.) TFv2 Cas9 with 20nt gRNA, 3.) SpyFi™ Cas9 with 18nt gRNA, 4.) TFv2 Cas9 with 18nt gRNA. TruCD33=truncated CD33 exon guide (18nt); CD33=full length CD33 exon 2 guide (20nt). TF=ThermoFisher® version 2 Cas9; Ald.SpyFi=SpyFi™ Cas9 protein from Aldevron®.

FIG. 16 illustrates off-target detection (with correction). Top row indicates the Cas9 protein used in the experiments. TF=ThermoFisher® version 2; Ald.SpyFi=SpyFi™ Cas9 protein from Aldevron®. Bottom row indicates the gRNA used. TruCD33=truncated CD33 exon guide (18nt); CD33=full length CD33 exon 2 guide (20nt). Timepoint indicates the duration of the experiment (only the Day 1 timepoint is thought to be biologically relevant).

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably +1%, and still more preferably +0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa and lambda light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

“Allogeneic” refers to any material derived from a different animal of the same species.

“Xenogeneic” refers to any material derived from an animal of a different species.

The term “chimeric antigen receptor” or “CAR,” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs has specificity to a selected target, for example a B cell surface receptor. CARs may also comprise an intracellular activation domain, a transmembrane domain and an extracellular domain comprising a tumor associated antigen binding region. In some aspects, CARs comprise an extracellular domain comprising an anti-B cell binding domain fused to CD3-zeta transmembrane and intracellular domain

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

When “an immunologically effective amount,” “an autoimmune disease-inhibiting effective amount,” or “therapeutic amount” is indicated, the precise amount of the compositions of the present invention to be administered can be determined by a physician or researcher with consideration of individual differences in age, weight, tumor size, extent of infection or metastasis, and condition of the patient (subject).

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “limited toxicity” as used herein, refers to the peptides, polynucleotides, cells and/or antibodies of the invention manifesting a lack of substantially negative biological effects, anti-tumor effects, or substantially negative physiological symptoms toward a healthy cell, non-tumor cell, non-diseased cell, non-target cell or population of such cells either in vitro or in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides compositions and methods that utilize optimized CD33− targeting guide RNAs. In certain embodiments, the invention provides chemically modified CD33-targeting guide RNAs.

### Compositions

In certain aspects, the invention provides a chemically modified CD33-targeting guide RNA (gRNA). In one embodiment, the gRNA comprises a crRNA portion comprising (i) a guide sequence capable of hybridizing to a target endogenous gene locus encoding for CD33, and (ii) a repeat sequence; and a tracrRNA portion comprising an anti-repeat sequence that is complementary to the repeat sequence, wherein the crRNA portion and/or the tracrRNA portion comprises one or more modified nucleotides.

As used herein, the term “guide RNA” or “gRNA” refers to any nucleic acid that promotes the specific association (or “targeting”) of an RNA-guided nuclease such as a Cas9 or variant thereof to a target sequence (e.g., an endogenous gene locus encoding for CD33) in a cell.

As used herein, a “guide sequence” refers to the nucleotide sequence of a gRNA, whether unimolecular or modular, that is fully or partially complementary to a target domain or target polynucleotide within a DNA sequence in the genome of a cell where editing is desired (e.g., an endogenous gene locus encoding for CD33). Guide sequences are typically 10-30 nucleotides in length, preferably 16-24 nucleotides in length (e.g., 16, 17, 18, 19, 20, 21, 22, 23, or 24 nucleotides in length. In certain embodiments, the guide sequence is 18 nucleotides in length. In certain embodiments, the guide sequence is 20 nucleotides in length.

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

In addition to guide sequences, gRNAs typically include a plurality of domains that influence the formation or activity of gRNA/Cas9 complexes. For example, the duplexed structure formed by first and secondary complementarity domains of a gRNA (also referred to as a repeat: anti-repeat duplex) interacts with the recognition (REC) lobe of Cas9 and may mediate the formation of Cas9/gRNA complexes (Nishimasu et al. Cell 156: 935-949 (2014); Nishimasu et al. Cell 162(2), 1113-1126 (2015), both incorporated by reference herein).

Along with the first and second complementarity domains, Cas9 gRNAs typically include two or more additional duplexed regions that are necessary for nuclease activity in vivo but not necessarily in vitro (Nishimasu 2015, supra). A first stem-loop near the 3′ portion of the second complementarity domain is referred to variously as the “proximal domain,” “stem loop 1” (Nishimasu 2014, supra; Nishimasu 2015, supra) and the “nexus” (Briner et al. Mol. Cell, 56(2), 333-339 (2014)). One or more additional stem loop structures are generally present near the 3′ end of the gRNA, with the number varying by species: S. pyogenes gRNAs typically include two 3′ stem loops (for a total of four stem loop structures including the repeat: anti-repeat duplex), while S. aureus and other species have only one (for a total of three). A description of conserved stem loop structures (and gRNA structures more generally) organized by species is provided in Briner 2014, supra, which is incorporated herein by reference. Additional details regarding guide RNAs generally may be found in WO2018026976A1, which is incorporated herein by reference.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); and Jinek et al. Science, 337(6096), 816-821 (2012); which are incorporated by reference herein.

The chemically modified guide RNAs of the present disclosure may possess improved in vivo stability, improved genome editing efficacy, and/or reduced immunotoxicity relative to unmodified gRNAs.

In certain embodiments, the crRNA portion of the gRNA comprises one or more modified nucleotides. In certain embodiments, the tracrRNA portion of the gRNA comprises one or more modified nucleotides. In certain embodiments, the crRNA portion and the tracrRNA portion of the gRNA comprises one or more modified nucleotides.

The gRNA nucleotides can comprise any type of modification known to one of ordinary skill in the art.

In certain embodiments, the one or more modified nucleotides comprise a modification of a ribose group, a phosphate group, or a combination thereof. In certain embodiments, the modification of the ribose group is selected from 2′-O-methyl, 2′-fluoro, 2′-deoxy, 2′-O-(2-methoxyethyl) (MOE), 2′-NH2, or a bicyclic nucleotide such as locked nucleic acid (LNA), 2′-(S)-constrained ethyl (S-cEt), constrained MOE, or 2′-0,4′-C-aminomethylene bridged nucleic acid (2′,4′-BNANC).

In certain embodiments, the modification of the phosphate group comprises a phosphorothioate, phosphonoacetate (PACE), thiophosphonoacetate (thioPACE), amide, triazole, phosphonate, or phosphotriester modification. In certain embodiments, nucleotides at positions 1-3 from the 5′ end of the crRNA portion comprise 2′-O-methyl modifications.

In certain embodiments, nucleotides at positions 2-4 from the 3′ end of the tracrRNA portion comprise 2′-O-methyl modifications. In certain embodiments, nucleotides at positions 1-4 from the 5′ end of the crRNA portion comprise phosphorothioate modifications. In certain embodiments, nucleotides at positions 1-4 from the 3′ end of the tracrRNA portion comprise phosphorothioate modifications.

In certain embodiments, the guide sequence comprises the nucleic acid sequence GUCAGUGACGGUACAGGA (SEQ ID NO:1). In certain embodiments, the guide sequence consists of the nucleic acid sequence GUCAGUGACGGUACAGGA (SEQ ID NO:1). In certain embodiments, the guide sequence comprises the nucleic acid sequence mG*mU*mC*AGUGACGGUACAGGA (SEQ ID NO: 2), wherein m indicates a 2′-O-methyl modification, and wherein * indicates a phosphorothioate modification. In certain embodiments, the guide sequence consists of the nucleic acid sequence mG*mU*mC*AGUGACGGUACAGGA (SEQ ID NO: 2), wherein m indicates a 2′-O-methyl modification, and wherein * indicates a phosphorothioate modification.

In certain embodiments, the guide sequence comprises the nucleic acid sequence GAGUCAGUGACGGUACAGGA (SEQ ID NO:16). In certain embodiments, the guide sequence consists of the nucleic acid sequence GAGUCAGUGACGGUACAGGA (SEQ ID NO:16). In certain embodiments, the guide sequence comprises the nucleic acid sequence mG*mA*mG*UCAGUGACGGUACAGGA (SEQ ID NO: 17), wherein m indicates a 2′-O-methyl modification, and wherein * indicates a phosphorothioate modification. In certain embodiments, the guide sequence consists of the nucleic acid sequence mG*mA*mG*UCAGUGACGGUACAGGA (SEQ ID NO: 17), wherein m indicates a 2′-O-methyl modification, and wherein * indicates a phosphorothioate modification.

Certain aspects of the invention provide a chemically modified CD33-targeting guide RNA comprising a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence, wherein the crRNA portion and/or the tracrRNA portion comprises one or more modified nucleotides.

In certain aspects, the invention also provides a CD33-targeting guide RNA comprising a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence.

In certain aspects, the invention also provides a chemically modified CD33-targeting guide RNA comprising a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence, wherein the crRNA portion and/or the tracrRNA portion comprises one or more modified nucleotides.

In certain aspects, the invention also provides a CD33-targeting guide RNA comprising a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence.

Also provided in the invention are populations of modified HSPCs. In certain embodiments, the HSPCs comprise an insertion and/or deletion in an endogenous gene locus encoding for CD33, wherein the insertion and/or deletion is mediated by a CD33-targeting guide RNA. In certain embodiments, the guide RNA comprises a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. In certain embodiments, the HSPCs comprise an insertion and/or deletion in an endogenous gene locus encoding for CD33, wherein the insertion and/or deletion is mediated by a CD33-targeting guide RNA. In certain embodiments, the guide RNA comprises a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence.

Another aspect of the invention includes a population of modified HSPCs, wherein the HSPCs comprise an insertion and/or deletion in an endogenous gene locus encoding for CD33. In certain embodiments, the insertion and/or deletion is mediated by a CD33-targeting guide RNA comprising a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. In certain embodiments, the insertion and/or deletion is mediated by a CD33-targeting guide RNA comprising a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence.

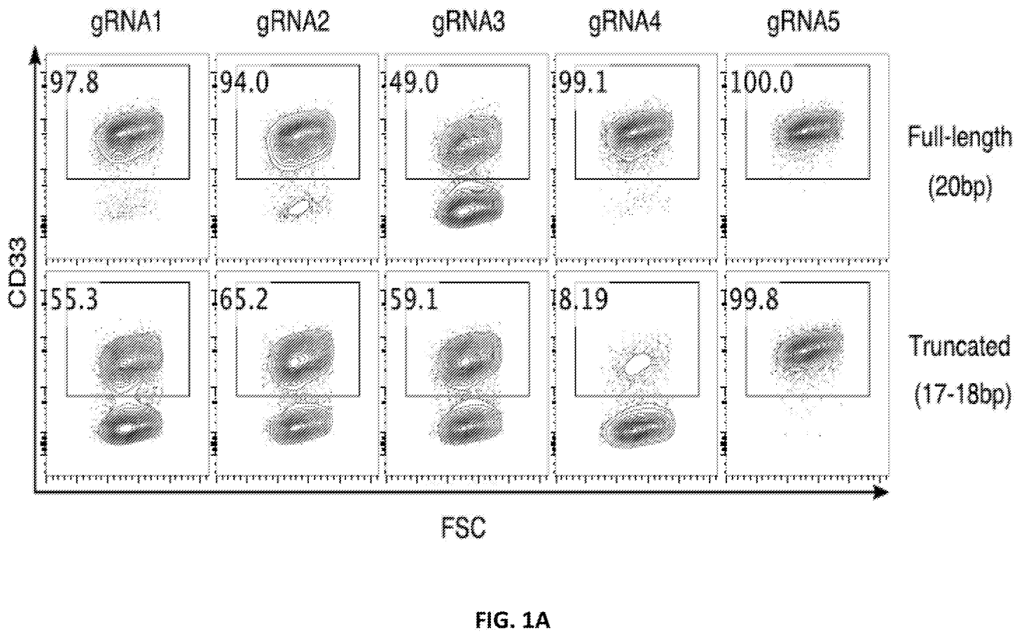

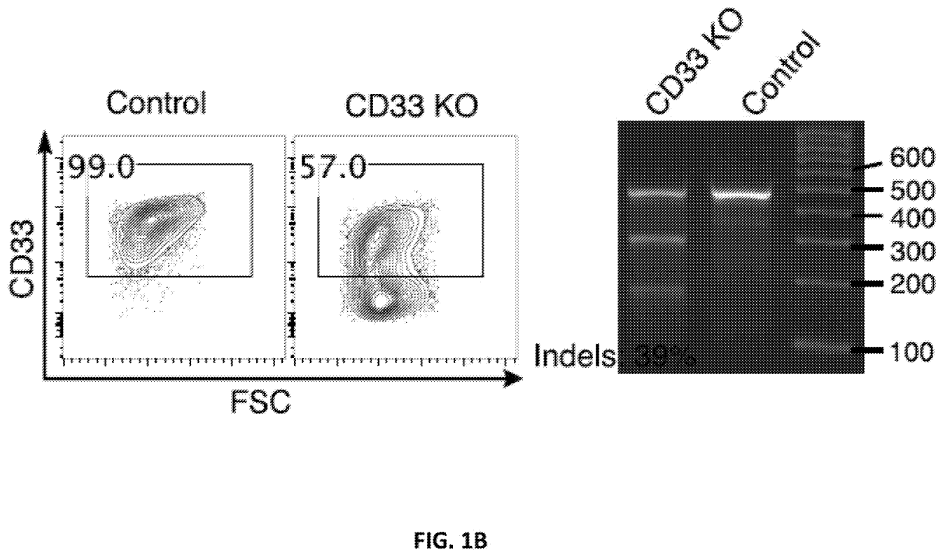

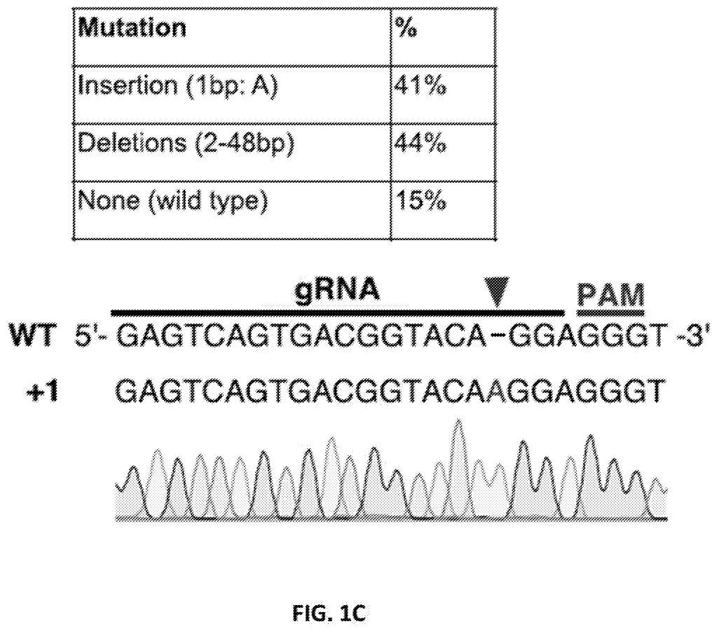

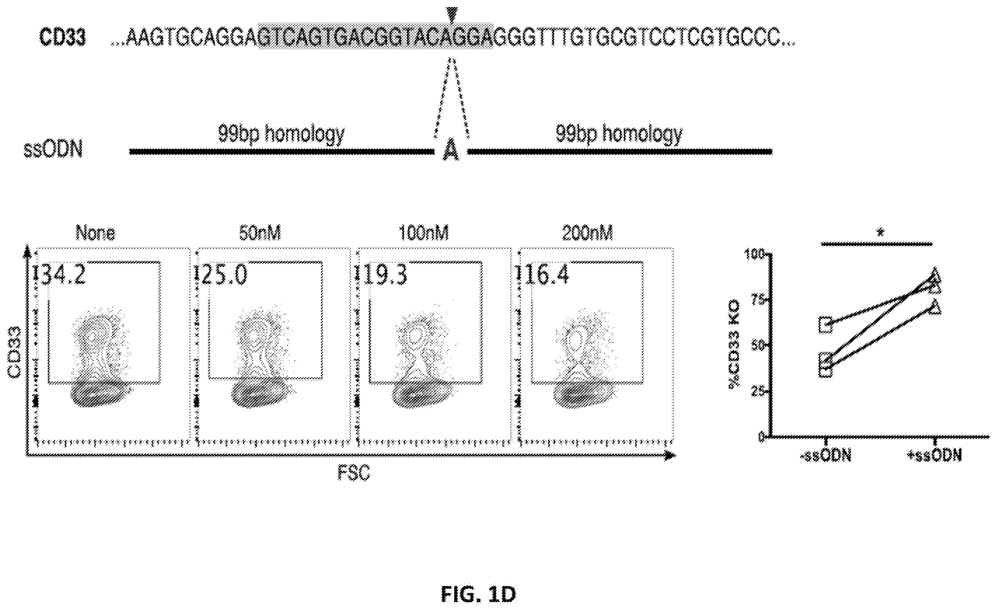

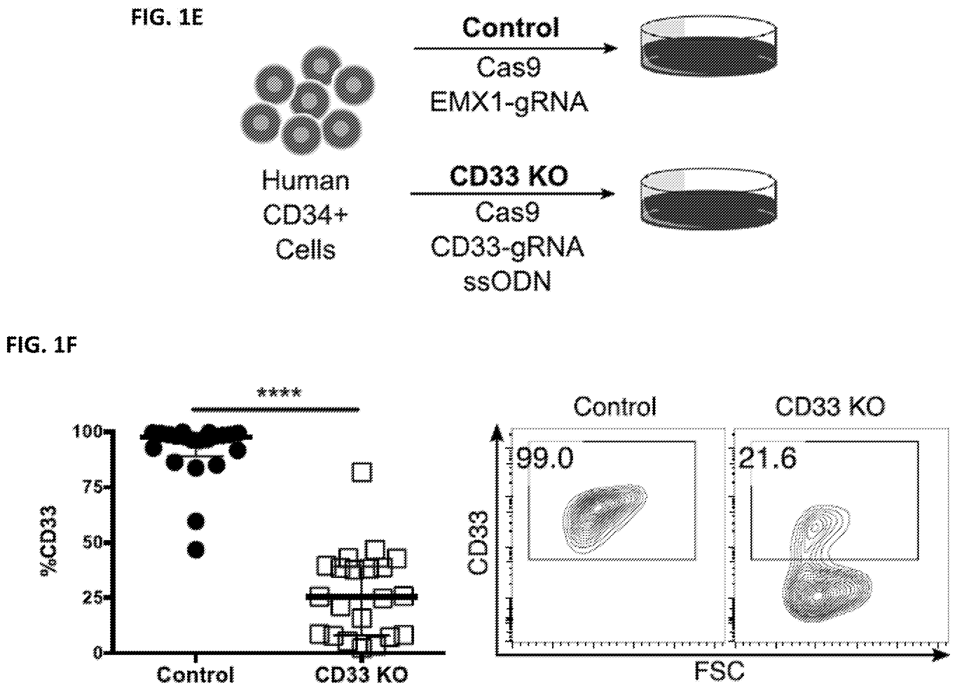

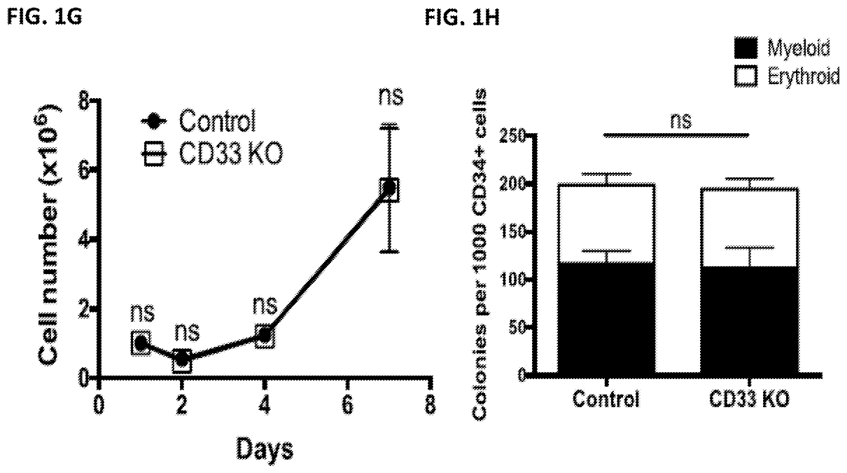

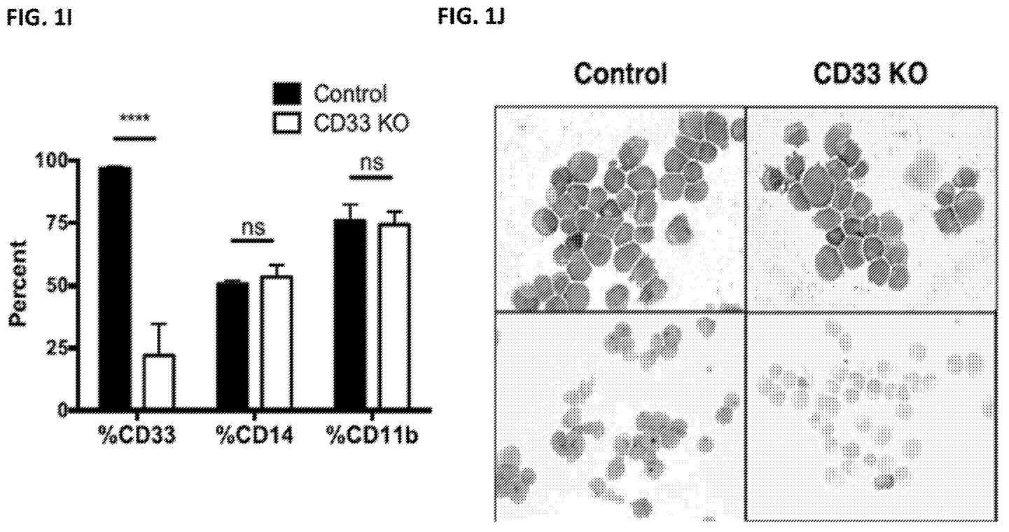

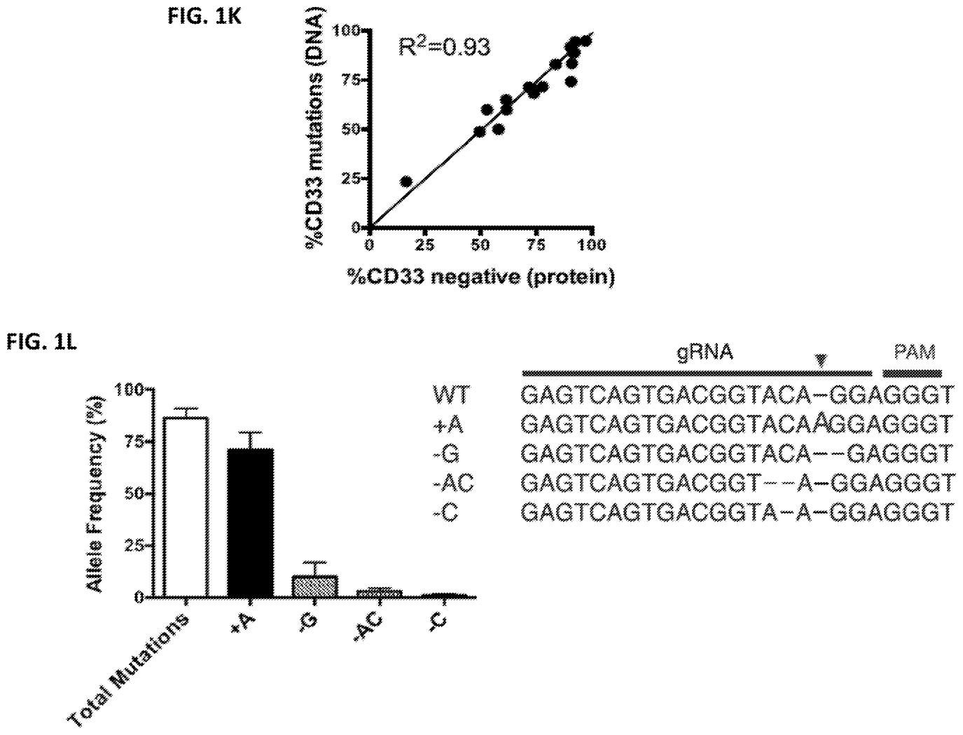

A proportion of modified cells may comprise a single adenine (A) nucleotide insertion (see, FIG. 1C). Accordingly, in certain embodiments, insertion and/or deletion in an endogenous gene locus encoding for CD33 may be enhanced by adding a single-stranded oligodeoxynucleotide (ssODN) template containing an adenine insertion. In certain embodiments, addition of the ssODN may result in a dose-dependent increase in the frequency of target locus modifications (e.g., leading to knock out of the target).

Accordingly, in certain embodiments, also mediating the insertion and/or deletion in an endogenous gene locus encoding for CD33 is a single-stranded oligonucleotide donor (ssODN) sequence.

In certain embodiments, the ssODN sequence comprises the nucleotide sequence GCAGGAGTCAGTGACGGTACAAGGAGGGTTTGTGCGTCC (SEQ ID NO:3). In certain embodiments, the ssODN comprises at least 10, at least 20, at least 30, or at least 35 contiguous nucleotides of SEQ ID NO: 3. In certain embodiments, the ssODN sequence comprises the nucleotide sequence GGTTGTCGGGCTGGGCCGAGCTGACCCTCGTTTCCCCACAGGGG CCCTGGCTATGGATCCAAATTTCTGGCTGCAAGTGCAGGAGTCAGTGACGGTACAAG GAGGGTTTGTGCGTCCTCGTGCCCTGCACTTTCTTCCATCCCATACCCTACTACGACA AGAACTCCCCAGTTCATGGTTACTGGTTCCGGGAAGGAGC (SEQ ID NO: 4). In certain embodiments, the ssODN comprises at least 30, at least 40, at least 50, at least 60, at least 70, at least 80, at least 90, at least 100, at least 110, at least 120, at least 130, at least 140, at least 150, at least 160, at least 170, at least 180, at least 190, at least 195, or at least 198 contiguous nucleotides of SEQ ID NO: 4. In certain embodiments, the ssODN sequence consists of the nucleotide sequence of SEQ ID NO: 4. The ssODN may encompass any and all variations in the length of the nucleotide sequence corresponding to SEQ ID NO: 4. The ssODN can comprises any stretch of nucleotides that are complementary to a target sequence (e.g. endogenous gene locus encoding for CD33) to the extent that homology directed repair (HDR) is possible.

Also provided is a population of modified HSPCs comprising an insertion and/or deletion in an endogenous gene locus encoding for CD33, wherein the insertion and/or deletion is mediated by a chemically modified CD33-targeting guide RNA. In certain embodiments, the guide RNA comprises a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprises one or more modified nucleotides, and the insertion and/or deletion is capable of downregulating gene expression of CD33.

In certain embodiments, the invention provides a population of modified HSPCs comprising an insertion and/or deletion in an endogenous gene locus encoding for CD33, wherein the insertion and/or deletion is mediated by a chemically modified CD33-targeting guide RNA. In certain embodiments, the guide RNA comprises a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprises one or more modified nucleotides, and the insertion and/or deletion is capable of downregulating gene expression of CD33.

In another aspect, the invention provides a population of HSPCs comprising an insertion and/or deletion in an endogenous gene locus encoding for CD33. In certain embodiments, the insertion and/or deletion is mediated by a chemically modified CD33-targeting guide RNA and a single-stranded oligonucleotide donor (ssODN) sequence. In certain embodiments, the guide RNA comprises a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprise one or more modified nucleotides, and the insertion and/or deletion is capable of downregulating gene expression of CD33. In certain embodiments, the ssODN sequence comprises the nucleotide sequence GCAGGAGTCAGTGACGGTACAAGGA GGGTTTGTGCGTCC (SEQ ID NO:3). In certain embodiments, the ssODN comprises at least 10, at least 20, at least 30, or at least 35 contiguous nucleotides of SEQ ID NO: 3. In certain embodiments, the ssODN sequence comprises the nucleotide sequence GGTTGTCGGGCTGGGCCGAGCTGACCCTCGTTTCCCCACAGGGG CCCTGGCTATGGATCCAAATTTCTGGCTGCAAGTGCAGGAGTCAGTGACGGTACAAG GAGGGTTTGTGCGTCCTCGTGCCCTGCACTTTCTTCCATCCCATACCCTACTACGACA AGAACTCCCCAGTTCATGGTTACTGGTTCCGGGAAGGAGC (SEQ ID NO: 4). In certain embodiments, the ssODN comprises at least 30, at least 40, at least 50, at least 60, at least 70, at least 80, at least 90, at least 100, at least 110, at least 120, at least 130, at least 140, at least 150, at least 160, at least 170, at least 180, at least 190, at least 195, or at least 198 contiguous nucleotides of SEQ ID NO: 4. In certain embodiments, the ssODN sequence consists of the nucleotide sequence of SEQ ID NO: 4.

In another aspect, the invention provides a population of HSPCs comprising an insertion and/or deletion in an endogenous gene locus encoding for CD33. In certain embodiments, the insertion and/or deletion is mediated by a chemically modified CD33-targeting guide RNA and a single-stranded oligonucleotide donor (ssODN) sequence. In certain embodiments, the guide RNA comprises a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprise one or more modified nucleotides, and the insertion and/or deletion is capable of downregulating gene expression of CD33. In certain embodiments, the ssODN sequence comprises the nucleotide sequence GCAGGAGTCAGTGACGGTACAAGGAGGGTTTGTGCGTCC (SEQ ID NO:3). In certain embodiments, the ssODN comprises at least 10, at least 20, at least 30, or at least 35 contiguous nucleotides of SEQ ID NO: 3. In certain embodiments, the ssODN sequence comprises the nucleotide sequence GGTTGTCGGGCTGGGCCGAGCTGACCCTCGTTTCCCCACAGGGG CCCTGGCTATGGATCCAAATTTCTGGCTGCAAGTGCAGGAGTCAGTGACGGTACAAG GAGGGTTTGTGCGTCCTCGTGCCCTGCACTTTCTTCCATCCCATACCCTACTACGACA AGAACTCCCCAGTTCATGGTTACTGGTTCCGGGAAGGAGC (SEQ ID NO: 4). In certain embodiments, the ssODN comprises at least 30, at least 40, at least 50, at least 60, at least 70, at least 80, at least 90, at least 100, at least 110, at least 120, at least 130, at least 140, at least 150, at least 160, at least 170, at least 180, at least 190, at least 195, or at least 198 contiguous nucleotides of SEQ ID NO: 4. In certain embodiments, the ssODN sequence consists of the nucleotide sequence of SEQ ID NO: 4.

In certain embodiments, the population of modified HSPCs comprises at least about 20%, 25%, 30%, 35%, 40%, 45%, 50%, 55%, 60%, 65%, 70%, 75%, 80%, 85%, 90%, or 95% modified HSPCs comprising the insertion and/or deletion in the endogenous gene locus encoding for CD33.

### CRISPR/Cas

In some embodiments, the invention employs the use of RNA-guided nucleases. Certain embodiments of the invention include cells that have been modified by a CRISPR/Cas system. CRISPR/Cas systems include, but are not limited to, the CRISPR/Cas9 system and the CRISPR/Cpf1 system. In certain embodiments, the invention includes cells that have been modified using the CRISPR/Cas9 system. In certain embodiments, the modifications include knocking-out or mutating an endogenous gene, e.g. CD33.

RNA-guided nucleases that may be employed by the invention include, without limitation, naturally-occurring Type II CRISPR nucleases such as Cas9, as well as other nucleases derived or obtained therefrom. Exemplary Cas9 nucleases that may be used in the present invention include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9). In certain embodiments, the RNA-guided nuclease is a Cas9 nuclease. In certain embodiments, the RNA-guided nuclease is an S. pyogenes Cas9 nuclease or an S. aureus Cas9 nuclease. In certain embodiments, the RNA-guided nuclease is a variant S. pyogenes Cas9 nuclease.

In certain embodiments, the RNA-guided nuclease is a variant S. pyogenes Cas9 nuclease comprising reduced off-target activity, and maintained or increased on-target activity relative to a wild-type Cas9 nuclease (e.g., a SpyFi™ Cas9 nuclease or a Alt-R HiFi Cas9 nuclease (e.g., a GMP-grade Alt-R HiFi Cas9 nuclease)). Such a variant S. pyogenes Cas9 nuclease exhibits improved specificity based on reduced off-target effects, while preserving high on-target activity. In certain embodiments, the RNA-guided nuclease is a variant Cas9 nuclease comprising reduced off-target activity, and maintained or increased on-target activity relative to a wild-type Cas9 nuclease, having an amino acid sequence set forth in SEQ ID NOs: 7-88 disclosed in PCT Publication No. WO2018/068053, the disclosure of which is herein incorporated by reference in its entirety. In certain embodiments, the RNA-guided nuclease is a variant Cas9 nuclease comprising reduced off-target activity, and maintained or increased on-target activity relative to a wild-type Cas9 nuclease, having an amino acid sequence set forth in SEQ ID NOs: 141-168 disclosed in PCT Publication No. WO2019/074542, the disclosure of which is herein incorporated by reference in its entirety. In certain embodiments, the RNA-guided nuclease is a SpyFi™ Cas9 nuclease. In certain embodiments, the RNA-guided nuclease is an Alt-R HiFi Cas9 nuclease. In certain embodiments, the RNA-guided nuclease is a GMP-grade Alt-R HiFi Cas9 nuclease.

In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus) or variation (e.g., full-length vs. truncated or split; naturally-occurring PAM specificity vs. engineered PAM specificity).

Various RNA-guided nucleases may require different sequential relationships between PAMs and protospacers. In general, Cas9s recognize PAM sequences that are 5′ of the protospacer as visualized relative to the top or complementary strand.

In addition to recognizing specific sequential orientations of PAMs and protospacers, RNA-guided nucleases generally recognize specific PAM sequences. S. aureus Cas9, for example, recognizes a PAM sequence of NNGRRT, wherein the N sequences are immediately 3′ of the region recognized by the gRNA targeting domain. S. pyogenes Cas9 recognizes NGG PAM sequences. It should also be noted that engineered RNA-guided nucleases can have PAM specificities that differ from the PAM specificities of similar nucleases (such as the naturally occurring variant from which an RNA-guided nuclease is derived, or the naturally occurring variant having the greatest amino acid sequence homology to an engineered RNA-guided nuclease).

In certain embodiments, the invention employs the use of the CRISPR/Cas9 system. The CRISPR/Cas9 system is a facile and efficient system for inducing targeted genetic alterations. Target recognition by the Cas9 protein requires a ‘seed’ sequence within the guide RNA (gRNA or sgRNA) and a conserved tri-nucleotide containing protospacer adjacent motif (PAM) sequence upstream of the gRNA-binding region. The CRISPR/Cas9 system can thereby be engineered to cleave virtually any DNA sequence by redesigning the gRNA for use in cell lines (such as 293T cells), primary cells, and CAR T cells. The CRISPR/Cas system can simultaneously target multiple genomic loci by co-expressing a single Cas9 protein with two or more gRNAs, making this system uniquely suited for multiple gene editing or synergistic activation of target genes.

One example of a CRISPR/Cas system used to inhibit gene expression, CRISPRi, is described in U.S. Publication No. US2014/0068797, which is incorporated herein by reference in its entirety. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR system comprises an expression vector, such as, but not limited to, an pAd5F35-CRISPR vector. In other embodiments, the Cas expression vector induces expression of Cas9 endonuclease. Other endonucleases may also be used, including but not limited to Cpf1, T7, Cas3, Cas8a, Cas8b, Cas10d, Cse1, Csy1, Csn2, Cas4, Cas10, Csm2, Cmr5, Fok1, other nucleases known in the art, and any combination thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

The guide nucleic acid sequence is specific for a gene and targets that gene for Cas endonuclease-induced double strand breaks. The sequence of the guide nucleic acid sequence may be within a loci of the gene. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length. In certain embodiments, the guide nucleic acid is 18 nucleotides in length. In certain embodiments, the guide nucleic acid is 20 nucleotides in length.

The guide nucleic acid sequence may be specific for any gene, e.g. CD33. The guide nucleic acid sequence includes a RNA sequence, a DNA sequence, a combination thereof (a RNA-DNA combination sequence), or a sequence with synthetic nucleotides. The guide nucleic acid sequence can be a single molecule or a double molecule. In one embodiment, the guide nucleic acid sequence comprises a single guide RNA.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional. In certain embodiments, the tracr sequence has at least 50%, 60%, 70%, 80%, 90%, 95% or 99% of sequence complementarity along the length of the tracr mate sequence when optimally aligned.

In other embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in US20110059502, incorporated herein by reference. In certain embodiments, a tagged CRISPR enzyme is used to identify the location of a target sequence.

Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Another delivery mode for the CRISPR/Cas9 comprises a combination of RNA and purified Cas9 protein in the form of a Cas9-guide RNA ribonucleoprotein (RNP) complex. (Lin et al., 2014, ELife 3:e04766). Viral vector delivery systems include DNA and RNA viruses, which have either episomal or integrated genomes after delivery to the cell (Anderson, 1992, Science 256:808-813; and Yu et al., 1994, Gene Therapy 1:13-26).

In certain embodiments, the CRISPR/Cas is derived from a type II CRISPR/Cas system. In other embodiments, the CRISPR/Cas system is derived from a Cas9 protein. The Cas9 protein can be from Streptococcus pyogenes, Streptococcus thermophilus, or other species. In certain embodiments, Cas9 can include: spCas9, Cpf1, CasY, CasX, or saCas9.

In general, CRISPR/Cas proteins comprise at least one RNA recognition and/or RNA binding domain. RNA recognition and/or RNA binding domains interact with the guiding RNA. CRISPR/Cas proteins can also comprise nuclease domains (i.e., DNase or RNase domains), DNA binding domains, helicase domains, RNAse domains, protein-protein interaction domains, dimerization domains, as well as other domains. The CRISPR/Cas proteins can be modified to increase nucleic acid binding affinity and/or specificity, alter an enzymatic activity, and/or change another property of the protein. In certain embodiments, the CRISPR/Cas-like protein of the fusion protein can be derived from a wild type Cas9 protein or fragment thereof. In other embodiments, the CRISPR/Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

### Gene Editing Systems

Certain aspects of the invention include gene editing systems for implementing the methods described herein. As used herein, the term “gene editing system” refers to any system that comprises RNA-guided gene editing activity (i.e., RNA-guided genome editing activity; RNA-guided DNA editing activity).

In certain embodiments, the gene editing system comprises: (a) any guide RNA as described herein, comprising a crRNA portion and a tracrRNA portion, wherein (i) the crRNA portion comprises a guide sequence capable of hybridizing to a target endogenous gene locus, and a repeat sequence; and (ii) the tracrRNA portion comprises an anti-repeat sequence that is complementary to the repeat sequence; and (b) any RNA-guided nuclease as described herein.

In certain embodiments, the gene editing system comprises: (a) a chemically modified CD33-targeting guide RNA as described herein, comprising a crRNA portion and a tracrRNA portion, wherein (i) the crRNA portion comprises a guide sequence capable of hybridizing to a target endogenous gene locus encoding for CD33, and a repeat sequence; and (ii) the tracrRNA portion comprises an anti-repeat sequence that is complementary to the repeat sequence; and (b) any RNA-guided nuclease as described herein.

In certain embodiments, the crRNA portion and/or the tracrRNA portion comprises one or more modified nucleotides.

In certain embodiments, the RNA-guided nuclease is a Cas9 nuclease. In certain embodiments, the Cas9 nuclease is an S. pyogenes Cas9 nuclease or an S. aureus Cas9 nuclease. In certain embodiments, the Cas9 nuclease is a variant S. pyogenes Cas9 nuclease. In certain embodiments, the variant Cas9 nuclease comprises reduced off-target activity, and maintained or increased on-target activity relative to a wild-type Cas9 nuclease (e.g., a SpyFi™ Cas9 nuclease or a Alt-R HiFi Cas9 nuclease (e.g., a GMP-grade Alt-R HiFi Cas9 nuclease)). In certain embodiments, the RNA-guided nuclease is a variant Cas9 nuclease comprising reduced off-target activity, and maintained or increased on-target activity relative to a wild-type Cas9 nuclease, having an amino acid sequence set forth in SEQ ID NOs: 7-88 disclosed in PCT Publication No. WO2018/068053, the disclosure of which is herein incorporated by reference in its entirety. In certain embodiments, the RNA-guided nuclease is a variant Cas9 nuclease comprising reduced off-target activity, and maintained or increased on-target activity relative to a wild-type Cas9 nuclease, having an amino acid sequence set forth in SEQ ID NOs: 141-168 disclosed in PCT Publication No. WO2019/074542, the disclosure of which is herein incorporated by reference in its entirety. In certain embodiments, the RNA-guided nuclease is a SpyFi™ Cas9 nuclease. In certain embodiments, the RNA-guided nuclease is an Alt-R HiFi Cas9 nuclease. In certain embodiments, the RNA-guided nuclease is a GMP-grade Alt-R HiFi Cas9 nuclease.

### Methods

Certain aspects of the invention include methods for generating a population of modified hematopoietic stem and progenitor cells (HSPCs). In certain embodiments, the method comprises introducing into the HSPCs a chemically modified CD33-targeting guide RNA, wherein the chemically modified guide RNA produces an insertion and/or deletion capable of downregulating gene expression of CD33.

Another aspect of the invention includes a method for generating a population of modified HSPCs comprising introducing into the HSPCs a CD33-targeting guide RNA comprising a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence.

In another aspect, the invention provides a method for generating a population of modified HSPCs comprising introducing into the HSPCs a chemically modified CD33-targeting guide RNA comprising: a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprises one or more modified nucleotides, and the chemically modified guide RNA produces an insertion and/or deletion capable of downregulating gene expression of CD33.

In another aspect, the invention provides a method for generating a population of modified HSPCs comprising introducing into the HSPCs a CD33-targeting guide RNA comprising a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence.

In another aspect, the invention provides a method for generating a population of modified HSPCs comprising introducing into the HSPCs a chemically modified CD33-targeting guide RNA comprising: a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprises one or more modified nucleotides, and the chemically modified guide RNA produces an insertion and/or deletion capable of downregulating gene expression of CD33.

Any one of the methods of the present invention may further comprise introducing into the HSPCs a homology-directed repair (HDR) template comprising a single-stranded oligonucleotide donor (ssODN) sequence. In certain embodiments, the ssODN sequence comprises (i) the nucleotide sequence GCAGGAGTCAGTGACGGTACAGGAGGGTTTGTGCGTCC (SEQ ID NO: 18) with an extra adenine inserted within the sequence, or (ii) the nucleotide sequence GCAGGAGTCAGTGACGGTACAAGGAGGGTTTGTGCGTCC (SEQ ID NO:3). In certain embodiments, the ssODN sequence consists of (i) the nucleotide sequence GCAGGAGTCAGTGACGGTACAGGAGGGTTTGTGCGTCC (SEQ ID NO: 18) with an extra adenine inserted within the sequence, or (ii) the nucleotide sequence GCAGGAGTCA GTGACGGTACAAGGAGGGTTTGTGCGTCC (SEQ ID NO:3). In certain embodiments, the ssODN comprises at least 10, at least 20, at least 30, or at least 35 contiguous nucleotides of SEQ ID NO: 3. In certain embodiments, the ssODN sequence comprises the nucleotide sequence GGTTGTCGGGCTGGGCCGAGCTGACCCTCGTTTCCCCACAGGGGCCCTGGCTATGGA TCCAAATTTCTGGCTGCAAGTGCAGGAGTCAGTGACGGTACAAGGAGGGTTTGTGC GTCCTCGTGCCCTGCACTTTCTTCCATCCCATACCCTACTACGACAAGAACTCCCCAG TTCATGGTTACTGGTTCCGGGAAGGAGC (SEQ ID NO: 4). In certain embodiments, the ssODN comprises at least 30, at least 40, at least 50, at least 60, at least 70, at least 80, at least 90, at least 100, at least 110, at least 120, at least 130, at least 140, at least 150, at least 160, at least 170, at least 180, at least 190, at least 195 or at least 198 contiguous nucleotides of SEQ ID NO: 4. In certain embodiments, the ssODN sequence consists of the nucleotide sequence of SEQ ID NO: 4.

Another aspect of the invention includes a method for generating a population of modified HSPCs comprising introducing into the HSPCs a CD33-targeting guide RNA comprising: a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. In certain embodiments, a single-stranded oligonucleotide donor (ssODN) is introduced into the HSPCs. In certain embodiments, the ssODN sequence comprises the nucleotide sequence GCAGGAGTCAGTGACGGTACAAGGAGGGTTTGTGCGTCC (SEQ ID NO:3). In certain embodiments, the ssODN comprises at least 10, at least 20, at least 30, or at least 35 contiguous nucleotides of SEQ ID NO: 3. In certain embodiments, the ssODN sequence comprises the nucleotide sequence of SEQ ID NO: 4. In certain embodiments, the ssODN comprises at least 30, at least 40, at least 50, at least 60, at least 70, at least 80, at least 90, at least 100, at least 110, at least 120, at least 130, at least 140, at least 150, at least 160, at least 170, at least 180, at least 190, at least 195 or at least 198 contiguous nucleotides of SEQ ID NO: 4. In certain embodiments, the ssODN sequence consists of the nucleotide sequence of SEQ ID NO: 4.

Yet another aspect of the invention includes a method for generating a population of modified HSPCs comprising introducing into the HSPCs a chemically modified CD33-targeting guide RNA. In certain embodiments, the guide RNA comprises a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprises one or more modified nucleotides. In certain embodiments, the chemically modified guide RNA produces an insertion and/or deletion capable of downregulating gene expression of CD33. In certain embodiments, a single-stranded oligonucleotide donor (ssODN) sequence comprising the nucleotide sequence GCAGGAGTCAGTGACGGTACAAGGAGGGTTTGTGCGTCC (SEQ ID NO:3) may be introduced into the HSPCs.

Still another aspect of the invention includes a method for generating a population of modified HSPCs comprising introducing into the HSPCs a chemically modified CD33-targeting guide RNA comprising: a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprises one or more modified nucleotides, and the chemically modified guide RNA produces an insertion and/or deletion capable of downregulating gene expression of CD33.

In another aspect, the invention provides a method for generating a population of modified HSPCs comprising introducing into the HSPCs a CD33-targeting guide RNA comprising: a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. In certain embodiments, a single-stranded oligonucleotide donor (ssODN) is introduced into the HSPCs. In certain embodiments, the ssODN sequence comprises the nucleotide sequence GCAGGAGTCAGTGACGGTACAAGGAGGGTTTGTGCGTCC (SEQ ID NO:3). In certain embodiments, the ssODN comprises at least 10, at least 20, at least 30, or at least 35 contiguous nucleotides of SEQ ID NO: 3. In certain embodiments, the ssODN sequence comprises the nucleotide sequence of SEQ ID NO: 4. In certain embodiments, the ssODN comprises at least 30, at least 40, at least 50, at least 60, at least 70, at least 80, at least 90, at least 100, at least 110, at least 120, at least 130, at least 140, at least 150, at least 160, at least 170, at least 180, at least 190, at least 195 or at least 198 contiguous nucleotides of SEQ ID NO: 4. In certain embodiments, the ssODN sequence consists of the nucleotide sequence of SEQ ID NO: 4.

In another aspect, the invention provides a method for generating a population of modified HSPCs comprising introducing into the HSPCs a chemically modified CD33-targeting guide RNA. The guide RNA comprises a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprises one or more modified nucleotides. In certain embodiments, the chemically modified guide RNA produces an insertion and/or deletion capable of downregulating gene expression of CD33. In certain embodiments, a single-stranded oligonucleotide donor (ssODN) sequence comprising the nucleotide sequence GCAGGAGTCAGTGACGGTACAAGGAGGGTTTGTGC GTCC (SEQ ID NO:3) may be introduced into the HSPCs.

In another aspect, the invention provides a method for generating a population of modified HSPCs comprising introducing into the HSPCs a chemically modified CD33-targeting guide RNA comprising: a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence In certain embodiments, the crRNA portion and/or the tracrRNA portion comprises one or more modified nucleotides, and the chemically modified guide RNA produces an insertion and/or deletion capable of downregulating gene expression of CD33.

In certain embodiments, the population of modified HSPCs comprise at least about 20%, 25%, 30%, 35%, 40%, 45%, 50%, 55%, 60%, 65%, 70%, 75%, 80%, 85%, 90%, 95% modified HSPCs comprising the insertion and/or deletion capable of downregulating gene expression of CD33.

The HSPCs may comprise autologous cells. In certain embodiments, the autologous cells are obtained from a source selected from the group consisting of peripheral blood mononuclear cells, cord blood cells, bone marrow, lymph node, and spleen. In certain embodiments, the HSPCs are CD34+ HSPCs.

In certain embodiments, the HSPCs are resistant to a CD33-targeted cell therapy. CD33-targeted cell therapies include, but are not limited to, CD33 CAR-T therapy. Examples of CD33 CARs and CAR-T therapy can be found for example in WO2017079400A1, PCT/CN2014/082589, U.S. Pat. No. 9,777,061B2, and US20160096892A1, which are incorporated by reference in their entireties herein.

### Methods of Treatment

The present invention includes methods for treating cancer in a subject in need thereof. In certain embodiments, the method comprises administering to a subject a population of hematopoietic stem and progenitor cells (HSPCs) that have been modified to become resistant to a CD33-targeting therapy. In certain embodiments, the CD33-targeting therapy may be an antibody that targets CD33, or a chimeric antigen receptor that targets CD33. Accordingly, in certain embodiments the present invention provides a method for treating cancer in a subject in need thereof, comprising administering to the subject a population of HSPCs that have been modified to become resistant to a CD33-targeting therapy, and further comprising administering to the subject a CD33-targeting therapy.

In certain embodiments, the CD33-targeting therapy is an anti-CD33 antibody. In certain embodiments, the CD33-targeting therapy comprises a CD33 CAR-T cell (e.g., a modified T cell comprising a chimeric antigen receptor comprising a CD33 antigen binding domain, a transmembrane domain, one or more costimulatory domains, and an intracellular signaling domain). Accordingly, in certain embodiments, the method comprises administering to the subject a CD33-targeted cell therapy comprising a CD33 CAR-T cell; and a population of hematopoietic stem and progenitor cells (HSPCs) that are resistant to the CD33-targeted therapy. In such embodiments, rendering a population of HSPCs resistant to a CD33-targeted therapy augments the CD33-targeting cell therapy.

In certain embodiments, the population of modified hematopoietic stem and progenitor cells (HSPCs) that are resistant to the CD33-targeted therapy comprise an insertion and/or deletion in an endogenous gene locus encoding for CD33, wherein the insertion and/or deletion is mediated by a CD33-targeting guide RNA. In certain embodiments, the guide RNA comprises a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. In certain embodiments, the insertion and/or deletion is capable of downregulating gene expression of CD33, and the method thereby treats cancer in the subject.

In certain embodiments, the population of modified hematopoietic stem and progenitor cells (HSPCs) that are resistant to the CD33-targeted therapy comprise an insertion and/or deletion in an endogenous gene locus encoding for CD33, wherein the insertion and/or deletion is mediated by a CD33-targeting guide RNA. In certain embodiments, the guide RNA comprises a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. In certain embodiments, the insertion and/or deletion is capable of downregulating gene expression of CD33, and the method thereby treats cancer in the subject.

In certain aspects, the invention includes a method of treating a cancer in a subject in need thereof, comprising administering to the subject a CD33-targeted cell therapy comprising a CD33 CAR-T cell a population of modified HSPCs that are resistant to the CD33-targeted therapy. In certain embodiments, the HSPCs comprise an insertion and/or deletion in an endogenous gene locus encoding for CD33. In certain embodiments, the insertion and/or deletion is mediated by a CD33-targeting guide RNA and a single-stranded oligonucleotide donor (ssODN) sequence. In certain embodiments, the guide RNA comprises a crRNA portion comprising (i) a guide sequence consisting of the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. In certain embodiments, the ssODN sequence comprises the nucleotide sequence GCAGGAGTCAGTGACGGTACAA GGAGGGTTTGTGCGTCC (SEQ ID NO:3). In certain embodiments, the insertion and/or deletion is capable of downregulating gene expression of CD33, and the method thereby treats cancer in the subject.

In another aspect, the invention includes a method of treating a cancer in a subject in need thereof, comprising administering to the subject a CD33-targeted cell therapy comprising a CD33 CAR-T cell and a population of modified HSPCs that are resistant to the CD33-targeted therapy. In certain embodiments, the HSPCs comprise an insertion and/or deletion in an endogenous gene locus encoding for CD33, wherein the insertion and/or deletion is mediated by a chemically modified CD33-targeting guide RNA. In certain embodiments, the guide RNA comprises a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprises one or more modified nucleotides. In certain embodiments, the insertion and/or deletion is capable of downregulating gene expression of CD33, and the method thereby treats cancer in the subject.

In yet another aspect, the invention includes a method of treating a cancer in a subject in need thereof, comprising administering to the subject a CD33-targeted cell therapy comprising a CD33 CAR-T cell and a population of modified HSPCs that are resistant to the CD33-targeted therapy. In certain embodiments, the HSPCs comprise an insertion and/or deletion in an endogenous gene locus encoding for CD33. In certain embodiments, the insertion and/or deletion is mediated by a chemically modified CD33-targeting guide RNA and a single-stranded oligonucleotide donor (ssODN) sequence. In certain embodiments, the guide RNA comprises a crRNA portion comprising (i) a guide sequence comprising the nucleic acid sequence GUCAGUGACGGUACAGGA (SEQ ID NO:1) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprises one or more modified nucleotides. In certain embodiments, the ssODN sequence comprises the nucleotide sequence GCAGGAGTCAGTGACGGTACAAGGAGGGTTTGTGCGTCC (SEQ ID NO:3). In certain embodiments, the insertion and/or deletion is capable of downregulating gene expression of CD33, thereby treating cancer in the subject. In certain embodiments, the ssODN comprises at least 10, at least 20, at least 30, or at least 35 sequential nucleotides of SEQ ID NO: 3. In certain embodiments, the ssODN sequence comprises the nucleotide sequence of SEQ ID NO: 4. In certain embodiments, the ssODN comprises at least 30, at least 40, at least 50, at least 60, at least 70, at least 80, at least 90, at least 100, at least 110, at least 120, at least 130, at least 140, at least 150, at least 160, at least 170, at least 180, at least 190, at least 195 or at least 198 sequential nucleotides of SEQ ID NO: 4. In certain embodiments, the ssODN sequence consists of the nucleotide sequence of SEQ ID NO: 4.

In another aspect, the invention includes a method of treating a cancer in a subject in need thereof, comprising administering to the subject a CD33-targeted cell therapy comprising a CD33 CAR-T cell a population of modified HSPCs that are resistant to the CD33-targeted therapy. In certain embodiments, the HSPCs comprise an insertion and/or deletion in an endogenous gene locus encoding for CD33. In certain embodiments, the insertion and/or deletion is mediated by a CD33-targeting guide RNA and a single-stranded oligonucleotide donor (ssODN) sequence. In certain embodiments, the guide RNA comprises a crRNA portion comprising (i) a guide sequence consisting of the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. In certain embodiments, the ssODN sequence comprises the nucleotide sequence GCAGGAGTCAGTGACGGTACAAGGAGGGTTTGTGCGTCC (SEQ ID NO:3). In certain embodiments, the insertion and/or deletion is capable of downregulating gene expression of CD33, and the method thereby treats cancer in the subject.

In another aspect, the invention includes a method of treating a cancer in a subject in need thereof, comprising administering to the subject a CD33-targeted cell therapy comprising a CD33 CAR-T cell and a population of modified HSPCs that are resistant to the CD33-targeted therapy. In certain embodiments, the HSPCs comprise an insertion and/or deletion in an endogenous gene locus encoding for CD33, wherein the insertion and/or deletion is mediated by a chemically modified CD33-targeting guide RNA. In certain embodiments, the guide RNA comprises a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprises one or more modified nucleotides. In certain embodiments, the insertion and/or deletion is capable of downregulating gene expression of CD33, and the method thereby treats cancer in the subject.

In another aspect, the invention includes a method of treating a cancer in a subject in need thereof, comprising administering to the subject a CD33-targeted cell therapy comprising a CD33 CAR-T cell and a population of modified HSPCs that are resistant to the CD33-targeted therapy. In certain embodiments, the HSPCs comprise an insertion and/or deletion in an endogenous gene locus encoding for CD33. In certain embodiments, the insertion and/or deletion is mediated by a chemically modified CD33-targeting guide RNA and a single-stranded oligonucleotide donor (ssODN) sequence. In certain embodiments, the guide RNA comprises a crRNA portion comprising (i) a guide sequence comprising the nucleic acid sequence GAGUCAGUGACGGUACAGGA (SEQ ID NO:16) capable of hybridizing to a target endogenous gene locus encoding for CD33, and (ii) a repeat sequence; and a tracrRNA portion comprising an anti-repeat sequence that is complementary to the repeat sequence. In certain embodiments, the crRNA portion and/or the tracrRNA portion comprises one or more modified nucleotides. In certain embodiments, the ssODN sequence comprises the nucleotide sequence GCAGGAGTCAGTGACGGTACAAGGAGGGTTTGTGCGTCC (SEQ ID NO:3). In certain embodiments, the insertion and/or deletion is capable of downregulating gene expression of CD33, thereby treating cancer in the subject. In certain embodiments, the ssODN comprises at least 10, at least 20, at least 30, or at least 35 sequential nucleotides of SEQ ID NO: 3. In certain embodiments, the ssODN sequence comprises the nucleotide sequence of SEQ ID NO: 4. In certain embodiments, the ssODN comprises at least 30, at least 40, at least 50, at least 60, at least 70, at least 80, at least 90, at least 100, at least 110, at least 120, at least 130, at least 140, at least 150, at least 160, at least 170, at least 180, at least 190, at least 195 or at least 198 sequential nucleotides of SEQ ID NO: 4. In certain embodiments, the ssODN sequence consists of the nucleotide sequence of SEQ ID NO: 4.

In certain embodiments, the HSPCs are administered to the subject prior to the CD33− targeted therapy. In certain embodiments, the HSPCs are administered to the subject after the CD33-targeted therapy. In certain embodiments, the HSPCs are administered to the subject at the same time as the CD33-targeted therapy.

In certain embodiments, the population of modified HSPCs comprise at least about 20%, 25%, 30%, 35%, 40%, 45%, 50%, 55%, 60%, 65%, 70%, 75%, 80%, 85%, 90%, 95% modified HSPCs comprising the insertion and/or deletion capable of downregulating gene expression of CD33.

The modified cells described herein may be included in a composition for therapy/treatment. The composition may include a pharmaceutical composition and further include a pharmaceutically acceptable carrier. A therapeutically effective amount of the pharmaceutical composition comprising the modified cells may be administered.

The modified cells described herein can be administered to a subject, preferably a mammal, even more preferably a human. In one embodiment, the modified cell differentiates into at least one blood cell type in the subject. In another embodiment, the modified cell is capable of self-renewal after administration into the subject.

In one embodiment, the condition is a cancer. Examples of various cancers include but are not limited to breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is a leukemia, such as acute myeloid leukemia (AML).

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

The cells of the invention to be administered may be autologous, allogeneic or xenogeneic with respect to the subject undergoing therapy. In certain embodiments, the HSPCs are autologous cells obtained from a source selected from the group consisting of peripheral blood mononuclear cells, cord blood cells, bone marrow, lymph node, and spleen. In certain embodiments, the HSPCs are CD34+ HSPCs.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

Once the cells are administered to the subject (e.g., human), the biological activity of the modified cells, in some embodiments, is measured by any of a number of known methods. Parameters to assess include specific binding of a modified or engineered or natural T cell or other immune cell to antigen, in vivo, e.g., by imaging, or ex vivo, e.g., by ELISA or flow cytometry. In certain embodiments, the ability of the modified cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107α, IFNγ, IL-2, and TNF. In some embodiments, the biological activity is measured by assessing clinical outcome, such as reduction in tumor burden or load. In some embodiments, toxic outcomes, persistence and/or expansion of the cells, and/or presence or absence of a host immune response, are assessed.

In some embodiments, the methods provided herein include designs to adapt the treatment to reduce and/or manage the risk of toxicity. In some embodiments, designs are included to reduce and/or manage the risk and/or development of a non-target mediated toxicity (e.g., cytokine release syndrome (CRS) or CAR-related encephalopathy syndrome (CRES)).

In certain embodiments, the methods provided herein further comprise monitoring the development of cytokine release syndrome resulting from the administration of a first dose of engineered cells (e.g., CAR-T cells). It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS) (grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the present disclosure provides for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

A subset of patients with CRS may manifest symptoms similar to macrophage activation syndrome (MAS) or hemophagocytic lymphohistiocytosis (HLH). Features consistent with macrophage activation syndrome (MAS) or hemophagocytic lymphohistiocytosis (HLH) have been observed in patients treated with CAR-T therapy, coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity. See, e.g., Namuduri and Brentjens, Expert Rev. Hematol. (2016) 9(6): 511-513, hereby incorporated by reference in its entirety.

In certain embodiments, the methods provided herein further comprise monitoring the development of immune cell-associated neurological toxicities resulting from the administration of a first dose of modified cells.

In some embodiments, immune cell-associated neurological toxicity includes CAR-related encephalopathy syndrome (CRES). Accordingly, the present disclosure provides for, following the diagnosis of CRES, appropriate CRES management strategies to mitigate the physiological symptoms of CRES. CRES management strategies are known in the art. For example, benzodiazepines (e.g., lorazepam) may be administered to control seizures that may arise from severe CRES-related impairment. Immune effector cell-associated neurotoxicity syndrome (ICANS) may manifest as delirium, encephalopathy, aphasia, lethargy, difficulty concentrating, agitation, tremor, seizures, and, rarely, cerebral edema. In addition, headache is very common and might not represent neurotoxicity per se. Previously considered in aggregate with CRS, neurotoxicity is now treated as a separate entity owing to its distinct timing and response to intervention. Neurologic symptoms may occur during or more commonly after CRS symptoms, vary among patients, and have an unclear pathophysiology, distinct from CRS. See, e.g., Lee et al. Biology of Blood and Marrow Transplantation (2019) 25(4): 625-538, hereby incorporated by reference in its entirety.

Mild to moderate cases of CRES are generally managed with supportive care (e.g., I.V. hydration and limiting oral intake), neurology evaluation and consultation (e.g., EEG, fundoscopic exam, brain/spine MRI), and tocilizumab and early corticosteroid therapy may be considered (e.g., 8 mg/kg I.V. maximum dose 800 mg Q8h of tocilizumab; and/or dexamethasone 10 mg I.V. Q6h). For severe CRES impairment, additional treatment with methylprednisolone (e.g., 1 mg/kg I.V. Q12h), and status epilepticus management may be required. For non-convulsive status epilepticus, lorazepam may be administered to control evidence of seizures (e.g., 0.5 mg I.V., with increased dosage by 0.5 mg increment to 2 mg I.V. total). For convulsive status epilepticus, 2 mg I.V. lorazepam may be administered to control seizures, with additional 2 mg I.V. as required. Maintenance dosing of levetiracetam, lorazepam and/or phenobarbital, and EEG may be considered for all status epilepticus.

### Introduction of Nucleic Acids

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the expanded cells, transfecting the expanded cells, and electroporating the expanded cells. One nucleic acid may be introduced by one method and another nucleic acid may be introduced into the cell by a different method.

RNA

In one embodiment, the nucleic acids introduced into the cell are RNA. In another embodiment, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a chimeric membrane protein. By way of example, the template encodes an antibody, a fragment of an antibody or a portion of an antibody. By way of another example, the template comprises an extracellular domain comprising a single chain variable domain of an antibody, such as anti-CD3, and an intracellular domain of a co-stimulatory molecule. In one embodiment, the template for the RNA chimeric membrane protein encodes a chimeric membrane protein comprising an extracellular domain comprising an antigen binding domain derived from an antibody to a co-stimulatory molecule, and an intracellular domain derived from a portion of an intracellular domain of CD28 and 4-1BB.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In some embodiments, the RNA is electroporated into the cells, such as in vitro transcribed RNA.

The disclosed methods can be applied to the modulation of T cell activity in basic research and therapy, in the fields of cancer, stem cells, acute and chronic infections, and autoimmune diseases, including the assessment of the ability of the genetically modified cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the invention is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Sources of HSCs or Progenitor Cells

Prior to expansion, a source of the cells is obtained from a subject. Non-limiting examples of subjects include humans, dogs, cats, mice, rats, non-human primates, swine and transgenic species thereof. Preferably, the subject is a human. The cells can be obtained from a number of sources, including peripheral blood mononuclear cells, bone marrow, cord blood, lymph node tissue, spleen tissue, umbilical cord, and tumors. In certain embodiments, a HSC or progenitor cell line available in the art, may be used. In certain embodiments, the cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, the cells are isolated from peripheral blood by lysing the red blood cells and depleting the monocytes, for example, by centrifugation through a PERCOLL™ gradient. Alternatively, the cells can be isolated from umbilical cord. In any event, a specific subpopulation of HSC or progenitor cells can be further isolated by positive or negative selection techniques.

The cord blood mononuclear cells so isolated can be depleted of cells expressing certain antigens, including, but not limited to, CD4, CD5, CD8, CD11b, CD14, CD19, CD24, CD45, CD56, and CD66b. Depletion of these cells can be accomplished using an isolated antibody, a biological sample comprising an antibody, such as ascites, an antibody bound to a physical support, and a cell bound antibody.

Enrichment of a cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD34+ cells by negative selection, a monoclonal antibody cocktail typically includes antibodies to CD4, CD5, CD8, CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

The cells can also be frozen after isolation, e.g. the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, the HSPCs are autologous cells. In certain embodiments, the cells are obtained from a source selected from the group consisting of peripheral blood mononuclear cells, cord blood cells, bone marrow, lymph nodes, and spleen. In certain embodiments, the cells are CD34+.

### Expansion of HSC or Progenitor Cells

The present invention includes a population of cells comprising the modified cell described herein. In one embodiment, the method for generating the modified cell described herein also includes expanding the cell or the modified cell. In one embodiment, the expansion is prior to the step of introducing the nucleic acid. In yet another embodiment, the expansion is prior to the step of introducing the nucleic acid. In some embodiments, the cells disclosed herein can be expanded by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the cells are expanded in the range of about 20 fold to about 50 fold.

The cells can be incubated in cell medium in a culture apparatus for a period of time or until the cells reach high cell density for optimal passage before passing the cells to another culture apparatus. The culturing apparatus can be of any culture apparatus commonly used for culturing cells in vitro. The cell medium may be replaced during the culture of the cells at any time. Preferably, the cell medium is replaced about every 2 to 3 days. The cells are then harvested from the culture apparatus whereupon the cells can be used immediately or cryopreserved to be stored for use at a later time. In one embodiment, the invention includes cryopreserving the expanded cells. The cryopreserved cells are thawed prior to introducing nucleic acids into the cell.

In another embodiment, the method further comprises isolating the cell and expanding the cell. In another embodiment, the invention further comprises cryopreserving the cell prior to expansion. In yet another embodiment, the invention further comprises thawing the cryopreserved cell prior to introducing the nucleic acids.

The culturing step as described herein (contact with agents as described herein) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore, the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for HSC or progenitor cell culture include an appropriate media (e.g., Minimal Essential Media or RPMI Media 1640 or, X-vivo 15, (Lonza)) that may contain factors necessary for proliferation and viability, such as but not limited to, serum (e.g., fetal bovine or human serum), GM-CSF, insulin, IFN-gamma, interleukin-1 (IL-1), IL-3, IL-4, IL-6, IL-7, IL-10, IL-12, IL-15, SCF, TGF-beta, TNF-α and TPO. or any other additives for the growth of cells known to the skilled artisan. In one embodiment, the cell culture includes IL-3, IL-6, GM-CSF, SCF and TPO. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, StemLine, StemSpan, with added amino acids, sodium pyruvate, and vitamins, either serum-free or supplemented with an appropriate amount of serum (or plasma) or a defined set of hormones, and/or an amount of cytokine(s) sufficient for the growth and expansion of HSC or progenitor cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). The medium used to culture the cells may include an agent that can stimulate the modified cells to expand.

### Pharmaceutical Compositions

Pharmaceutical compositions of the present invention may comprise the modified cell as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The cells of the invention to be administered may be autologous, allogeneic or xenogeneic with respect to the subject undergoing therapy.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It can generally be stated that a pharmaceutical composition comprising the modified cells described herein may be administered at a dosage of 10⁴ to 10⁹ cells/kg body weight, in some instances 10⁵ to 10⁶ cells/kg body weight, including all integer values within those ranges. Cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the modified cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

Cell lines: The Molm14 cell line was obtained from Deutsche Sammlung von Mikroorganismen und Zellkulturen (DSMZ); the cell line was established from a 20 year old male. Cells were cultured in RPMI media with 10% fetal calf serum, penicillin and streptomycin. Molm14 cells were lentivirally transduced with luciferase/GFP under the control of the EFla promoter and sorted to 100% GFP+ for mouse experiments.

The LCL8664 rhesus macaque cell line was obtained from the American Type Culture Collection (ATCC); it was derived from a 5 year old male. Cells were cultured in RPMI media with 10% fetal calf serum, penicillin and streptomycin.

Primary human CD34+ cells: G-CSF mobilized peripheral blood samples were obtained from left-over clinical specimens according to a University of Pennsylvania IRB-approved consent form for clinical hematopoietic stem/progenitor cell donation. Human subjects were de-identified and thus age and sex information are not available. CD34+ selection was performed using the CD34 Microbead Kit (Miltenyi Biotec, 130-046-702), and purity was confirmed by flow cytometry to be >95%. Cells were cultured in StemSpan SFEM (Stem Cell Technologies, 09650) supplemented with human cytokines (SCF 100 ng/ul, Flt3 ligand 100 ng/ul, TPO 50 ng/ul, IL-6 50 ng/ul, all purchased from Peprotech).

Primary human T cells: Autologous peripheral blood mononuclear cells remaining from the G-CSF mobilized peripheral blood donors after CD34+ cell selection were expanded in vitro using anti-CD3/CD28 Dynabeads (Life Technologies) in X-Vivo 15 media with 5% human serum, penicillin, streptomycin and glutamax. T cells were transduced with lentivirus containing the CAR33 construct as previously described (Kenderian et al., (2015) Leukemia 29, 1637-1647). The human CD33 scFv was derived from the gemtuzumab ozogamicin antibody (clone P67.6), and the light and heavy chains were cloned into the murine CART19 plasmid vector (Milone et al., (2009) Mol. Ther. 17, 1453-1464), a third generation lentiviral vector in which the CAR is expressed under control of the EF-1 alpha promoter. The CAR construct uses a light-to-heavy orientation of the scFv, followed by the human CD8 hinge and transmembrane, 4-1BB costimulatory domain and CD3zeta intracellular signaling domain. Lentivirus was generated by transient transfection of 293T cells using Lipofectamine 2000 (ThermoFisher Scientific®, Cat #11668500). T cells were activated with CD3/CD28 Dynabeads (ThermoFisher Scientific®, Cat #11132D) at a 1:3 ratio, followed by transduction with lentivirus 1 day after stimulation at a multiplicity of infection of 3. T cells were grown for 10-14 days prior to cryopreservation. Prior to all experiments T cells were thawed and rested at 37° C. for 4-16 hours.

Xenogeneic mouse transplantation model: Male and female 8-12 week old NOD-SCID-IL2rγ−/− (NSG) mice were purchased from Jackson laboratories or bred in-house. Mice were maintained in dedicated BSL-2 animal barrier spaces. Age and sex-matched animals were randomly assigned to experimental groups; no gender-specific influences were detected in the experimental results.

1-5×10⁵ control or gene-edited human CD34+ cells were infused by tail vein injection into the mice after prior conditioning with busulfan 30 mg/kg intraperitoneally. Mice were evaluated by serial retro-orbital bleeding. Mice were also injected with Molm14 (1×10⁶ cells) and/or autologous CAR T cells (5×10⁶ cells) after CD34+ cell engraftment was confirmed (range: 4-12 weeks post-CD34+ cell injection). After Molm14 injection, mice underwent weekly bioluminescent imaging using a Xenogen IVIS-200 Spectrum camera. Images were acquired and analyzed using Living Image version 4.4 (Caliper LifeSciences, PerkinElmer).

Guide RNA design and production: Human codon optimized Cas9 expressed under the T7 promoter was provided, and Cas9 mRNA was in vitro transcribed using the mMessage mMachine T7 Ultra kit (Ambion, AM1345). Cas9 protein was purchased from PNA Bio (CP02). The human CD33-targeting gRNA was obtained by screening the top 5 gRNAs previously reported to have high KO efficiency for CD33 (Doench et al., (2014) Nat. Biotechnol. 32, 1262-1267.). Full length and truncated gRNA sequences are depicted in Table 1.

TABLE 1

| | Full length | | Truncated | |
|---|---|---|---|---|
| gRNA1 | tggggtgattatgagcaccg | (SEQ ID NO: 5) | ggtgattatgagcaccg | (SEQ ID NO: 6) |
| gRNA2 | tgagcatcgtagacgccagg | (SEQ ID NO: 7) | gcatcgtagacgccagg | (SEQ ID NO: 8) |
| gRNA3 | atccctggcactctagaacc | (SEQ ID NO: 9) | gcctggcactctagaacc | (SEQ ID NO: 10) |
| gRNA4 | gagtcagtgacggtacagga | (SEQ ID NO: 11) | gtcagtgacggtacagga | (SEQ ID NO: 12) |
| gRNA5 | tgtcacatgcacagagagct | (SEQ ID NO: 13) | gcacatgcacagagagct | (SEQ ID NO: 14) |

The control EMX1-targeting gRNA was previously reported (Cong et al., (2013) Science 339, 819-823); sequence is as follows: 5′-GAGTCCGAGCAGAAGAAGAA-3′ (SEQ ID NO: 15). All gRNAs were generated using the pUC57-sgRNA expression vector (Addgene plasmid #51132) using overlapping DNA oligonucleotides to generate the gRNA insert; cloning was performed using standard molecular biology techniques. The gRNAs were in vitro transcribed using the TranscriptAid T7 High Yield Transcription Kit (Thermo Fisher Scientific®, #K0441). RNA was purified using the RNeasy Mini Kit (QIAGEN, 74104). Truncated gRNA4 was found to be most efficacious and was used for all subsequent experiments.

Single-stranded oligodeoxynucleotide (ssODN) design: A ssODN was added to the electroporation to increase the frequency of knockout mutations. The ssODN was designed to have 99 base pair homology arms around the Cas9 cut site of the CD33 gene, and an A nucleotide insertion that generates a frameshift mutation and premature truncation of the CD33 protein. The ssODN was ordered from IDT (Integrated DNA Technologies) as an 4 nmole Ultramer DNA Oligo. Lyophilized oligo was resuspended in 40 ul TE buffer and 1 ul was used for each electroporation of 2×10⁶ cells in 500 ul OptiMEM.

Human CD33 locus DNA analysis: Genomic DNA was extracted from the CD34+ cells using the High Pure PCR Template Preparation Kit (Roche, 11796828001). Surveyor Mutation Detection kit (Transgenomics) was used to detect the presence of mutations and band intensities were analyzed using ImageJ software. PCR amplicons were analyzed by Sanger sequencing and allele modification frequency was calculated using TIDE (Tracking of Indels by Decomposition) software available at https://tide.nki.nl.

Rhesus macaque CD33 locus DNA analysis: Genomic DNA from hematopoietic cells was extracted using the DNeasy Blood & Tissue kit (QIAGEN, Germantown, Md.). Deletions between gRNAs E2 and E3 were detected by PCR amplification using F1 and R2, then quantified with a digital droplet PCR (ddPCR) (Bio-Rad) following the manufacturer's instructions. Briefly, genomic DNA from neutrophils was digested by HindIII (NEB) and diluted to 5 ng/ul. Each reaction included 10 mL of 2×ddPCR Supermix for probes (nodUTP), 1 mL of CD33 deletion primer/probe mix (FAM), 1 mL of CD33 distal primer/probe mix (HEX), and 30-100 ng of genomic DNA. The samples were processed using the QX200 Droplet Digital PCR system, and the CD33 deletion rate was calculated with the QuantaSoft software (Bio-Rad).

Cell line electroporation: An AMLcell line, Molm1, was used for human gRNA screening. Cells were washed once and resuspended in Opti-MEM. Cas9 mRNA 10 ug was added to cell suspension and electroporated with the BTX ECM 830 Square Wave Electroporation System (Harvard Apparatus) using a single pulse of 400V and 5 msec. Cells were incubated at 32° C. overnight and re-electroporated with gRNA 5 ug the next day using the same machine and settings. Cells were kept at 32° C. until the following day, after which they were cultured at 37° C. until analysis.

Human CD34+ cell electroporation: Cas9 protein and gRNA were mixed and incubated at room temperature for 10 minutes prior to electroporation. CD34+ cells were washed and resuspended in Opti-MEM and electroporated with the Cas9 ribonucleoprotein complex using the BTX ECM 830 Square Wave Electroporation System (Harvard Apparatus) with a single pulse of 400V and 5 msec. 10 ug of Cas9 and 5 ug of gRNA was used for 2×10⁶ CD34+ cells in 750 ul total volume. After electroporation, cells were incubated at 32° C. until the following day, after which they were cultured at 37° C. until analysis.

In vitro human CD34+ cell differentiation: CD34+ cells were cultured in IMDM+10% FBS and the following human cytokines: SCF 100 ng/ul, Flt3 ligand 100 ng/ul, TPO 50 ng/ul, IL-6 50 ng/ul, GM-CSF 100 ng/ul, IL-3 10 ng/ul (all purchased from Peprotech). Cells were maintained at 0.5-1×10⁶ cells/ml for 5-7 days prior to analysis.

Morphological analysis: Cytospin specimens of in vitro differentiated control or CD33 KO human CD34+ cells were prepared and immunocytochemistry for CD33 was performed using an anti-CD33 antibody (Novocastra NCL-L-CD33). Staining was done on a Leica Bond-III™ instrument using the Bond Polymer Refine Detection System (Leica Microsystems DS9800). Heat-induced epitope retrieval was done for 20 minutes with ER1 solution (Leica Microsystems AR9961). Incubation with the anti-CD33 antibody was performed at 1:150 dilution for 15 min followed by 8 min post-primary step and 8 min incubation with polymer HRP. Endogenous peroxidase was blocked for 5 min, followed by 10 min incubation with DAB (3,3′-diaminobenzidine). All procedures were performed at room temperature. Slides are washed three times between each step with bond wash buffer or water.

Human colony forming cell (CFC) assay: One day after electroporation, 1000 CD34+ cells were plated in 1.1 ml of methylcellulose (MethoCult H4435 Enriched, Stem Cell Technologies) on 6 well plates in duplicate and cultured for two weeks, after which colonies were counted and scored. Individual colonies were picked and heat lysed in 40 ul of lysis buffer containing 50 mM NaOH and 0.2 mM EDTA. Samples were heated to 95° C. for 20 minutes then cooled down, after which 1 ul of 1M TrisCl was added. 2 ul of reaction was used for PCR with AccuPrime Pfx SuperMix (Invitrogen, 12344-040) as per manufacturer's instructions. Alternatively, MethoCult wells were solubilized with RPMI media overnight and flow cytometry was performed on single-cell suspensions.

Flow cytometry: In vitro differentiated CD34+ cells were analyzed by flow cytometry 5-7 days after electroporation using the following antibodies: CD11b-FITC (BioLegend, 301329), CD14-APC (BD, 340436), CD33-PE (Thermo Fisher Scientific®, 12-0339-41), CD45-BV421 (BioLegend, 304032), and Live/Dead Fixable Aqua (Life Technologies, L34957).

For in vivo studies assessing human hematopoietic engraftment, the following panel was used: mouse CD45-APC/Cy7 (BioLegend, 103116), human CD45-BV421 (BioLegend, 304032), Live/Dead Fixable Aqua (Life Technologies, L34957), CD19-PE/Cy7 (BioLegend, 115520), CD3-BV605 (BioLegend, 317322), CD11b-FITC (BioLegend, 301329), CD14-APC (BD, 340436), CD33− PE (Thermo Fisher Scientific®, 12-0339-41). For the in vivo studies using Molm14-GFP/luciferase, CD11b-FITC was removed from the panel. For the in vivo studies looking at the effects of G-CSF stimulation, CD66b-PerCP/Cy5.5 (BioLegend, 305107) and CD56-BV711 (BioLegend, 318335) were added. Countbright beads (Invitrogen) were used to quantify cell numbers.

Analysis of bone marrow human HSPC subsets was performed using the following panel: mouse CD45-APC/Cy7 (BioLegend, 103116), human CD45-BV421 (BioLegend, 304032), Live/Dead Fixable Aqua (Life Technologies, L34957), lineage-FITC (Thermo Fisher Scientific®, 22-7778-72), CD34-APC (BioLegend, 343608), CD38-BV711 (BioLegend, 303528), CD33-PE (Thermo Fisher Scientific®, 12-0339-41). All flow cytometry was performed on a BD Fortessa, and analysis was performed using FlowJo X10.

Intracellular cytokine assay of human myeloid cells: Cells were incubated with either monensin (BioLegend, 420701) or monensin and LPS for 4 hours at 37° C. Subsequently, cells were harvested and surface staining with CD33-PE was performed, followed by fixation with Invitrogen Fixation Medium A (GAS001S5) for 15 minutes. Cells were then incubated for 20 minutes with Permeabilization Medium B (GAS002S5) containing the following antibodies: MIP1b-PE/Cy7 (BD PharMingen, 560687), IL-8-AF488 (BioLegend, 511412), and TNFa-AF700 (BioLegend, 502928).

Phagocytosis assay: In vitro differentiated control or CD33 KO human myeloid cells were incubated with pHrodo green E. coli bioparticles (Thermo Fisher Scientific®, P343666) for 1 hour at 37° C. Cells were then washed once with Flow Buffer (PBS with 1% FBS and 0.1% sodium azide) and stained with CD33-PE (Thermo Fisher Scientific®, 12-0339-41) and Live/Dead Fixable Aqua (Life Technologies®, L34957) for 15 minutes at room temperature. Cells were then washed again with Flow Buffer and acquired by flow cytometry to quantify phagocytosis.

Cellular reactive oxygen species (ROS) analysis: In vitro differentiated control or CD33 KO human myeloid cells were stained with CellROX Green Flow Cytometry Assay Kit and evaluated for ROS by flow cytometry according to the manufacturer's protocol (Thermo Fisher Scientific®, C10492). In brief, cells were incubated with PMA (5 ng/ul) for 15 minutes at 37° C., after which the CellROX reagent was added at a final concentration of 500 nM. Cells were further incubated for 30 minutes at 37° C., then washed once with Flow Buffer and stained with CD33-PE (Thermo Fisher Scientific®, 12-0339-41) and Live/Dead Fixable Aqua (Life Technologies, L34957) for 15 minutes at room temperature. Cells were then washed again with Flow Buffer, and flow cytometry was performed to quantify ROS.

In vivo functional studies of xenografted human HSPC: NSG mice engrafted with human CD34+ cells were injected with G-CSF 300 ug/kg intraperitoneally twice daily for 2 days at 8 weeks post-transplantation. Frequency of human CD66b+ cells (neutrophils) and CD14+ cells (monocytes) were analyzed before and after G-CSF treatment. At 12 weeks post-transplantation, mice were given 10 ug of LPS intraperitoneally, and serum was collected at 0 and 3 hours after injection. Levels of human inflammatory cytokines were measured by BD cytometric bead array, Human Inflammatory Cytokine Kit (BD Biosciences, 551811).

Cytogenetics of human hematopoietic cells: Primary human CD34+ cells electroporated with Cas9 and either control or CD33-targeted gRNA and cultured for 7 days in IMDM+10% FBS with the following human cytokines: SCF 100 ng/ul, Flt3 ligand 100 ng/ul, TPO 50 ng/ul, IL-6 50 ng/ul, GM-CSF 100 ng/ul, IL-3 10 ng/ul. Samples were then treated with colcemid (Life Technologies, 15210-040) and hypotonic solution (homemade solution of EGTA 0.4 g (Sigma #4378), HEPES buffer powder 9.6 g (Life Technologies, 11344-025), KCl 6.0 g in 2 L of distilled water; pH adjusted to 7.4 with 10 mM NaOH) for 55 minutes, and fixed with a 3:1 methanol/acetic acid solution. Slides were prepared and G-banding was performed using trypsin-Wright staining (Trypsin-EDTA: Life Technologies, 15400-054; Wright stain stock solution: add 3 g wright stain powder (Sigma, WO625-25 g) to 1 L methanol; Wright stain working solution: add 1 ml stock solution to 3 ml pHydrion 6.8 buffer working solution). Metaphases were analyzed using Cytovision chromosome analysis software (Leica Bio-systems) and the results were described according to the International System for Human Cytogenetic Nomenclature (ISCN 2016). A clone is defined by conventional cytogenetics as the presence of at least two cells with the same chromosome gain or structural rearrangement, or at least three cells with the same chromosome loss.

Mass cytometry analysis of human hematopoietic cells: In vitro differentiated control or CD33 KO human HSPC were generated from three normal donors and cultured in cytokine-deplete media (1:32 dilution of differentiation media with cytokine-free IMDM) overnight prior to the assay. Cells were incubated with cisplatin at 50 mM concentration for 1 minute at room temperature for viability staining, then washed and resuspended in cytokine-deplete media. Cells were divided into 10 groups and following cytokines were added to each: none (basal), TPO (50 ng/ml), G-CSF (20 ng/ml), GM-CSF (20 ng/ml), IFNa (5000 U/ml), IFNg (20 ng/ml), IL-4 (20 ng/ml), IL-6 (50 ng/ml), PMA/ionomycin (50 nM and 1 ug/ml each), LPS (100 ng/ml). Cells were incubated at 37° C. for 15 minutes after which they were immediately fixed by addition of 1/10th volume of 16% paraformaldehyde (PFA) for 10 minutes at room temperature. Cells were then snap frozen in PBS with 0.5% FBS, 0.02% sodium azide and 10% DMSO and stored at −80° C.

Cells were thawed at 4° C. and then washed once with cell staining media (CSM; 1×PBS with 0.5% BSA and 0.02% sodium azide). Cells were then barcoded in groups of 20 cell samples from each donor (2 million cells from each of the 10 stimulation conditions for the control and CD33 KO cells) using a previously described protocol (Zunder et al., (2015) Nat. Protoc 10, 316-333). Briefly, cells were washed twice in PBS at 4° C. followed by a 15 minute incubation with palladium barcoding reagent (Fluidigm); the barcoding incubation (and all subsequent steps unless otherwise noted) were performed at room temperature. This barcoding protocol permanently labels each of the 20 samples with a unique combination of 3 of the 6 stable Pd isotopes which allows the cells from each sample to be identified. All 20 samples from each donor were then washed three times in CSM and combined into a single tube for antibody staining and mass cytometry analysis. Mass cytometry staining of the 40 million combined cells was performed in a staining volume of 2 mL for 50 minutes at room temperature with gentle agitation using separate staining steps for the surface antigens and intracellular antigens. Antibodies were either purchased from Fluidigm with metal tags attached, or were metal tagged using the Fluidigm MaxParX8 polymer kit according to the manufacturer's protocol. After each staining step, cells were washed twice in CSM. After surface staining, cells were fixed with 1.5% PFA and then permeabilized with 100% methanol for 15 minutes at −20° C. prior to the intracellular stain. After completion of intracellular staining cells were washed twice in CSM and then incubated in Irridium intercalator solution (PBS with a 1:5,000 dilution of the iridium intercalator pentamethylcyclopentadienyl-Ir(III)-dipyridophenazine [Fluidigm] and 1.5% PFA) at 4° C. for 16-72 hours prior to analysis.

Prior to analysis excess intercalator solution was removed with one CSM wash and two washes in pure water. Cells were then resuspended in pure water at a concentration of approximately 1 million per mL and mixed with mass cytometry measurement standard beads (Fluidigm). Cell events were acquired on a Helios mass cytometer (Fluidigm) at an event rate of 100 to 300 events per second with instrument-calibrated dual-count detection (Ornatsky et al., (2008) J. Anal. At. Spectrom. 23, 463-469). Instrument setting were as follows: noise reduction was on, event duration of 8 to 150, lower convolution threshold of 600, sigma=3, dual count start=1. After data acquisition, the mass bead signal was used to correct short-term signal fluctuation during the course of each experiment, and bead events were removed (Finck et al., (2013) Cytometry A 83, 483-494). An average of approximately 800,000 cell events were collected for each sample (range 568,274-1,029,292 events).

Mass cytometry analysis was performed using Cytobank; (Kotecha et al., (2010) Curr. Protoc. Cytom. Chapter 10, Unit10.17). SPADE analysis (Qiu et al., (2011) Nat. Biotechnol. 29, 886-891) was performed using Cytobank on a sample of approximately 40% of total events (to facilitate computational analysis). Down-sampling was performed to a target 8% of total events, clustering was performed to a target of 100 nodes, and fold change was calculated relative to the average of the basal condition of the control cells from the three donors. Approximate immunophenotypic annotations are indicated on each SPADE tree and assignment was performed manually based on a best approximation of the cell immunophenotype and signaling responses; given the artificial nature of the in vitro differentiation, the annotations represent a best estimation and may not be perfectly accurate. Mass cytometry data are plotted on an arcsinh transformed scale.

Rhesus macaque HSPC electroporation and autologous transplantation: Guide RNAs and Cas9 protein were mixed and incubated for 10 minutes before electroporation. Rhesus macaque CD34+ cells were washed with phosphate buffered saline (PBS) and resuspended in Opti-MEM. Subsequently, the cell suspension was mixed with the Cas9 RNP, then electroporated using the BTX ECM 830 Square Wave Electroporation System (Harvard Apparatus) with a single pulse of 400V for 5 msec. 30-60 ug of Cas9 protein and 15-30 ug of gRNA were used for electroporation of aliquots of 3-5×10⁶ CD34+ cells in a total volume of 750 ul. After electroporation, cells were pooled and incubated in X-VIVO™ 10 (Lonza, BE04-743Q) supplemented with 1% HSA (Baxter, 2G0012) and cytokines (SCF 100 ng/mL, FLT3L 100 ng/mL and TPO 100 ng/mL; all purchased from PeproTech) at 32° C. until the following day, when they were reinfused into the autologous macaque following total body irradiation (500 rad/day for 2 days).

Rhesus macaque peripheral blood flow cytometric analysis: Rhesus macaque PB was processed via centrifugation over Lymphocyte Separation Medium (MP Biomedicals, 0850494X). PBMNC (peripheral blood mononuclear cells) and neutrophils were separated and treated with ACKlysing buffer (Quality Biological, 118-156101). Cells were stained with the following antibodies: CD45-BV605 (BD Biosciences®, 564098), CD45-APC (BD Biosciences®, 561290), CD11b-FITC (BioLegend®, 301329), CD14-pacific blue (Thermo Fisher Scientific®, MHCD1428), CD3-BV786 (BD Biosciences, 563918), CD20-APC-cy7 (BD Biosciences, 335794), CD56-PE (BD Biosciences®, 555516), CD16-APC (BioLegend®, 302012), and CD33-PE (Miltenyi Biotec®, 130-091-732). Flow cytometric analysis and/or sorting were performed on a FACSARIA-II flow sorter (BD), and data were analyzed using FlowJo software (Tree-Star®).

Rhesus macaque colony forming unit (CFU) assay: Single CD34+ cell-derived CFUs were grown for 14 days in MethoCult H4435 Enriched (STEMCELL Technologies). For infusion product CFUs, CD34+ cells electroporated with Cas9 RNP were incubated for 24 hours at 32° C., then plated with 1.1 ml of MethoCult H4435 in 35-mm CFU dishes at a density of 2000 cells/dish. For ZL33's 4.5 m BM CFUs, 20 mL of BM aspirate was collected, and CD34+ cells were immunoselected as previously described (Donahue et al., (2005) Curr. Protoc. Immunol Chapter 22, Unit 22A.1; Wu et al., (2014) Cell Stem Cell 14, 486-499). BM-derived CD34+ cells were plated with 1.1 ml of MethoCult H4435 in 35-mm CFU dishes at a density of 2000 cells/dish. The remaining cells were used for targeted deep sequencing. Single CFU colonies were picked, and DNA was extracted with the Maxwell 16 Cell LEV DNA Purification Kit (Promega, AS1140) on day 14 of plating.

Rhesus bone marrow immunohistochemistry: Rhesus macaque bone marrow trephine biopsies were fixed in B-Plus fixative, decalcified, embedded in paraffin, and processed for morphologic evaluation using standard procedures. Bone marrow sections were stained with hematoxylin and eosin (H&E), and immunohistochemistry for MPO (Ventana Medical Systems, 760-2659) was performed using the Ventana Benchmark Ultra platform (Ventana Medical Systems). Images were using an Olympus BX-41 microscope (Olympus America) equipped with a DPlan 10/50 numeric aperture objectives and captured using an Olympus DP70 digital camera system.

Rhesus macaque neutrophil apoptosis and necrosis analysis: RM neutrophils were stained with Annexin V-PE and evaluated for apoptosis by flow cytometry according to the manufacturer's protocol (BD, 559763). Briefly, cells were washed with PBS and stained with 5 mL of Annexin V-PE in 1× binding buffer for 15 minutes at room temperature in the dark. For the ViViD assay, 1×10⁶ cells were washed with PBS and resuspended in 1 ml of PBS. 1 mL of reconstituted fluorescent reactive dye (Thermo Fisher Scientific®, L34964) was added to the cell suspension, and the cells were incubated on ice for 30 minutes in the dark. Cells were then washed with PBS, and apoptotic/necrotic cells were determined using a FACSARIA-II flow sorter (BD).

Rhesus macaque neutrophil chemotaxis assay: Neutrophil chemotaxis assay was performed on freshly isolated CD33+ and CD33− cells using the EZ-TAXIScan (Effector Cell Institute, Tokyo, Japan). In brief, 5×10³ neutrophils were added to the “Cell” well of the EZ-TAXIScan and 1 mL of either buffer or fMLF (5×10-8 M) was added to the opposing “Chemoattractant” well. Images of cellular migration were captured every 30 s for 60 min at 37° C. and ten randomly selected cells were tracked digitally using the ImageJ plug-in, MTrackJ. The paths of the migrating cells were plotted with the position of each cell at t=0 anchored at the origin. Using the coordinates of the individual cells in each image, the distance that each cell migrated and the average velocity were calculated using the distance formula.

Rhesus macaque phagocytosis assay: CD33 KO HSPC-transplanted rhesus macaques peripheral blood cells were incubated with pHrodo green E. coli bioparticles (Thermo Fisher Scientific®, P343666) for 1 hour at 37° C. Cells were washed once and stained with CD33-PE for 15 minutes at room temperature. Cells were then washed again and acquired by flow cytometry to quantify phagocytosis.

RNA-sequencing of human hematopoietic cells: RNA was isolated from 5 matched samples of in vitro differentiated control/CD33 KO human HSPC using Ambion RiboPure RNA purification kit (Thermo Fisher Scientific®, AM1924). RNA-seq libraries were generated using TruSeq RNA library prep kit (Illumina®, RS-122-2001/2) per the manufacturer's instructions. The libraries were sequenced as 100 bp single end using a HiSeq 2500 sequencer (Illumina®). Sequencing reads were aligned to the human genome (hg19) using RUM, and differential expression was analyzed using edgeR.

Off-target evaluation: In silico sites: Off-targets sites were selected based on two web tools: crispr.mit.edu and CCTop (https://crispr.cos.uni-heidelberg.de/). The top 5 coding sites and the top 3 non-coding sites from each web tool that were predicted to have off-target activity based on sequence homology to the CD33-targeting gRNA were selected. These off-target loci were PCR amplified from genomic DNA extracted from CD34+ cells treated with CRISPR/Cas9, using 5 matched control and CD33 KO donors. PCR products were designed to be 385+24 bp around the gRNA cut site. PCR products from each donor were pooled and libraries were generated using NEBnext. Sequencing was performed on the MiSeq 500 (Illumina) with 250 bp paired end reads. Point mutations and INDELs were identified through the in-house pipeline. Raw reads (fastq) were mapped to the human reference genome (hg19) using Burrows-Wheeler Aligner (BWA). Low complexity regions (LCRs) were removed from the mapped reads using bedtools and then realigned for the INDEL region by GATK IndelRealigner. GATK BaseRecalibrator was then use to recalibrate the filtered reads based on quality. Point mutations and INDELs were called by the variant caller, VarScan2 limited to the mutations that had an allele frequency >0.1%, a minimum read depth >8 and existence only in CD33 KO samples. The variants were then annotated by the ANNOVAR. Sequences with indels of >1 bp located within 5 bp of the predicted Cas9 cut site on either side were considered CRISPR/Cas9 induced genome modifications.

CIRCLE-seq: Genomic DNA was isolated using Gentra Puregene Kit (QIAGEN) according to manufacturer's instructions. CIRCLE-seq was performed as previously described (Tsai et al., (2017) Nat. Methods 14, 607-614). Briefly, purified genomic DNA was sheared with a Covaris E200 instrument to an average length of 300 bp. The fragmented DNA was end repaired, A tailed and ligated to a uracil-containing stem-loop adaptor, using KAPA HTP Library Preparation Kit PCR Free (KAPA Biosystems). Adaptor ligated DNA was treated with Lambda Exonuclease (NEB) and E. coli Exonuclease I (NEB) and then with USER enzyme (NEB) and T4 polynucleotide kinase (NEB). Intramolecular circularization of the DNA was performed with T4 DNA ligase (NEB) and residual linear DNA was degraded by Plasmid-Safe ATP-dependent DNase (Epicenter). In vitro cleavage reactions were performed with 125 ng of Plasmid-Safe-treated circularized DNA, 90 nM of SpCas9 protein (NEB), Cas9 nuclease buffer (NEB) and 90 nM of in vitro transcribed gRNA, in a 50 mL volume. Cleaved products were A tailed, ligated with a hairpin adaptor (NEB), treated with USER enzyme (NEB) and amplified by PCR with barcoded universal primers NEBNext Multiplex Oligos for Illumina (NEB), using Kapa HiFi Polymerase (KAPA Biosystems). Libraries were quantified by droplet digital PCR (Bio-Rad) and sequenced with 150 bp paired-end reads on an Illumina MiSeq instrument. CIRCLE-seq data analyses were performed using open-source CIRCLE-seq analysis software (https://github.com/tsailabSJ/circleseq).

Off-target evaluation: CIRCLE-seq sites: The top 7 sites with the highest CIRCLE-seq read count (c10orf20, DGKG, EPS8, ALDH3B1, RN7SL601P, DAPK2, TSPAN2) and all sites with 3 or less mismatches to the on-target site were selected to validate in primary CD33 KO cell samples. PCR amplification, sequencing and analysis was performed in a similar fashion to the in silico predicted off-target evaluation.

Quantification and statistical analysis: All statistical analyses were performed using GraphPad Prism 6. Unpaired Student's t test was performed for pairwise comparisons and one-way analysis of variance (ANOVA) with Bonferroni's multiple comparison post-test for three or more groups, as indicated.

The results of the experiments are now described.

### Example 1: Human CD33 KO HSPC Remain Functional

The CD33 gene was disrupted in primary human CD34+ cells using truncated guide RNAs (gRNAs). The truncated gRNAs increased the efficacy of CD33 knock-out (KO) compared to their full-length counterparts (FIG. 1A). Initially, around 40% KO in primary CD34+ cells was achieved (FIG. 1), of which a large proportion of mutations consisted of a single adenine (A) nucleotide insertion (FIG. 1C). Editing was further enhanced by adding a single-stranded oligodeoxynucleotide (ssODN) template containing the A insertion, leading to dose-dependent increase in frequency of KO (FIG. 1D). Subsequent experiments were performed via transfection of Cas9 protein complexed with a truncated gRNA targeting CD33 and the ssODN template to generate CD33 KO HSPCs, while control HSPCs were treated with Cas9 and a gRNA targeting the irrelevant gene EMX1 (FIG. 1E). In vitro differentiated CD33 KO HSPCs had markedly reduced levels of surface CD33 protein expression (27%±4%) compared to controls (92%±3%) (FIG. 1F) without impairment of growth or differentiation (FIGS. 1G-1J). High frequency of mutations in the CD33 gene was confirmed by tracking of indels by decomposition (TIDE) and next-generation sequencing (FIGS. 1K-1L).

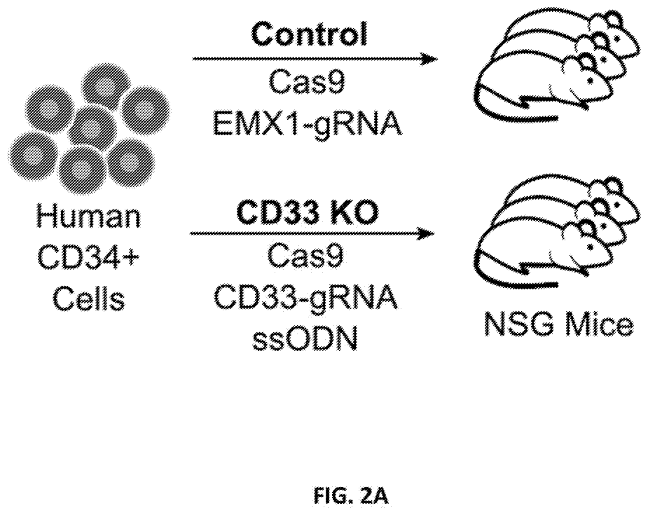

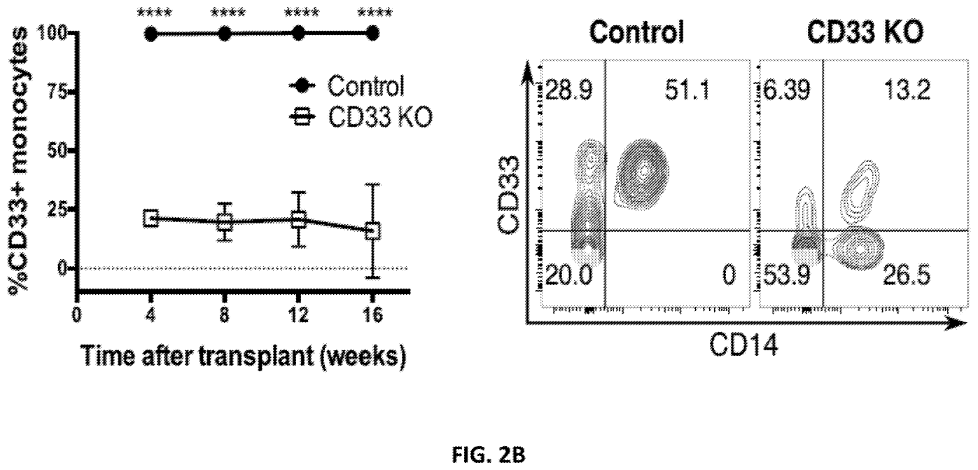

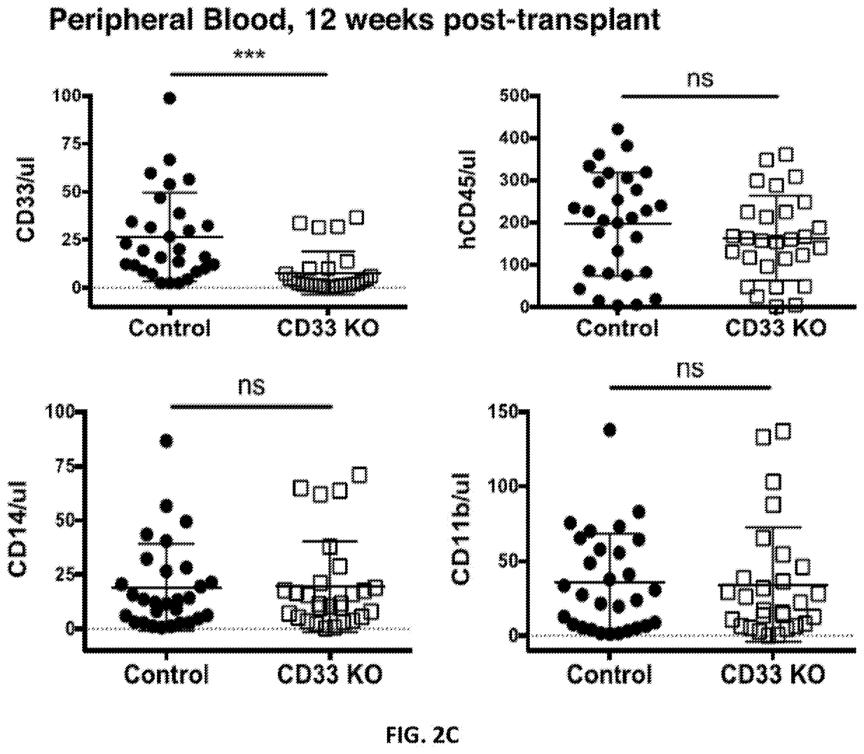

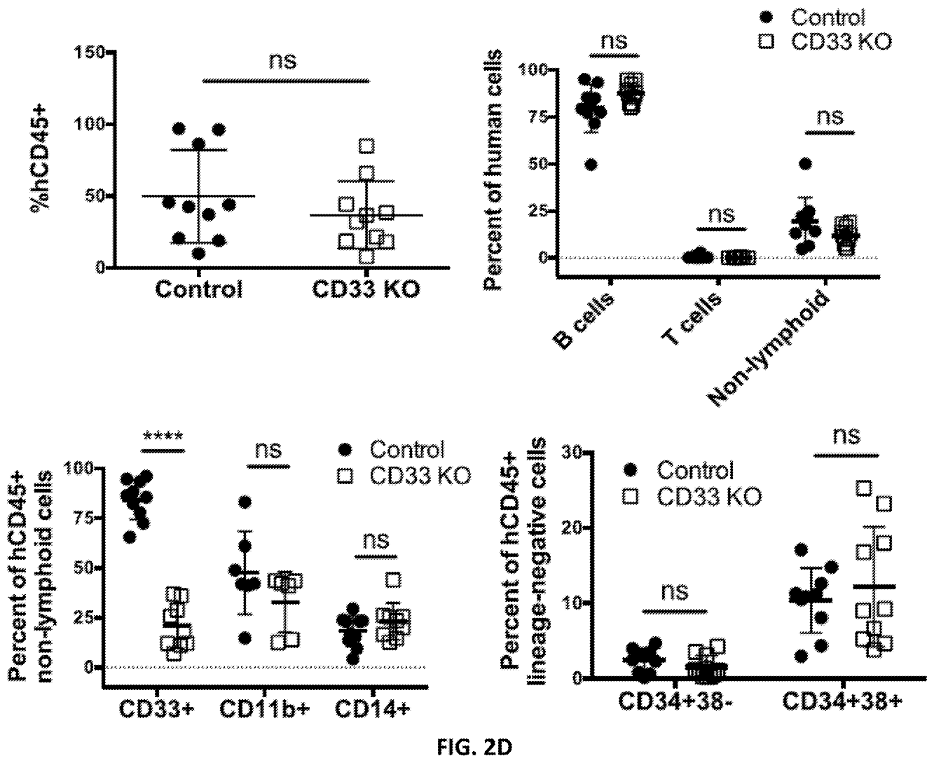

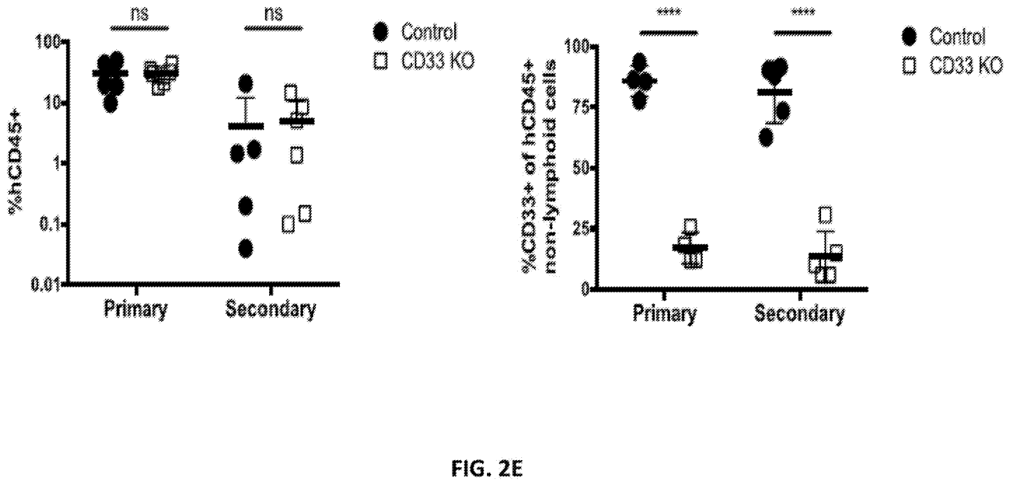

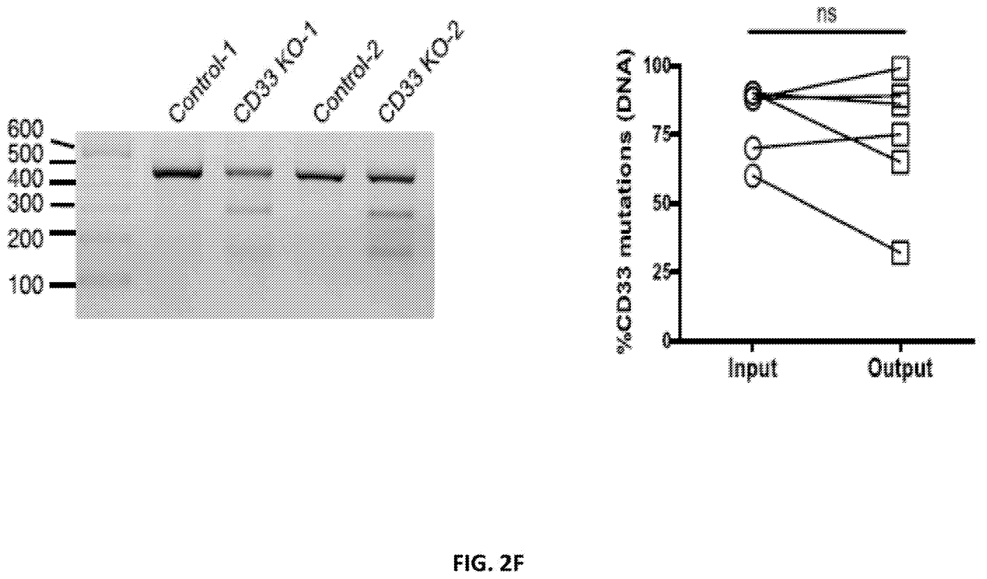

NOD/SCID/IL2rγ^{null} (NSG) mice were injected with control (Cas9/EMX1-gRNA) or CD33 KO (Cas9/CD33-gRNA/ssODN) HSPCs and human cell engraftment was followed in the peripheral blood over time (FIG. 2A). Human monocytes in the peripheral blood of mice engrafted with CD33 KO HSPC had diminished expression of CD33 that was sustained over time (FIG. 2B). Total human CD33+ cells were significantly decreased in the CD33 KO HSPC-engrafted mice compared to controls, while numbers of total human CD45+ cells, as well as myeloid subsets (CD11b+ and CD14+ cells), were identical between the two groups (FIG. 2C). Bone marrow analysis confirmed similar levels of human engraftment and differentiation between control and CD33 KO HSPC-engrafted mice, while decreased expression of CD33 was observed in myeloid cells. Levels of the most primitive (CD34+38−) and more mature (CD34+38+) human HSPCs in the marrow were not significantly different between the two groups (FIG. 2D). To document that CD33 KO HSPCs retain long-term repopulating function, bone marrow cells were harvested from mice engrafted with control or CD33 KO HSPCs after 16 weeks and these cells were then transferred into secondary recipients. Bone marrow analysis of the secondary recipients after 12 additional weeks showed sustained human engraftment, while CD33 expression remained diminished in CD33 KO HSPC recipients (FIG. 2E), and PCR confirmed the presence of mutations in CD33 at similar levels to the initial infusion product (FIG. 2F). These results demonstrated that CD33 KO occurred in cells capable of long-term and serial engraftment.

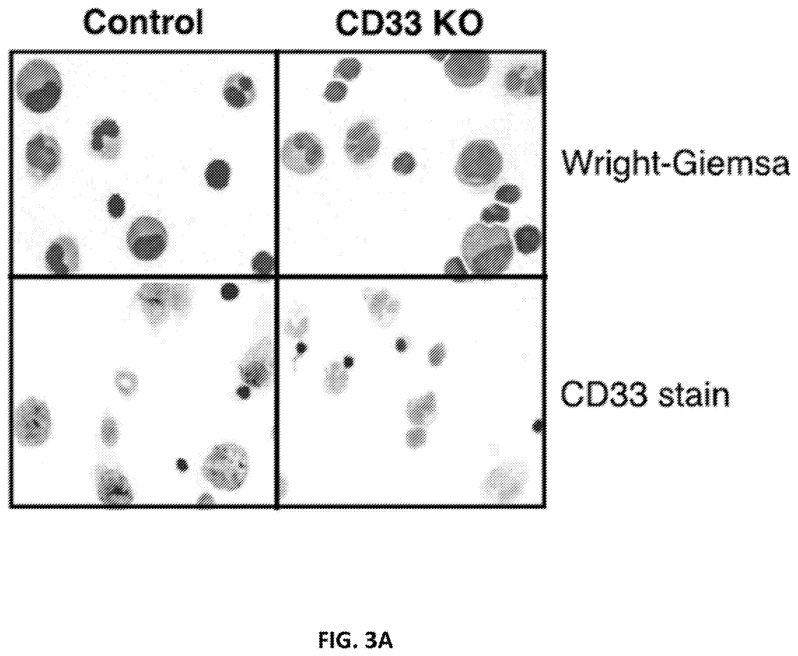

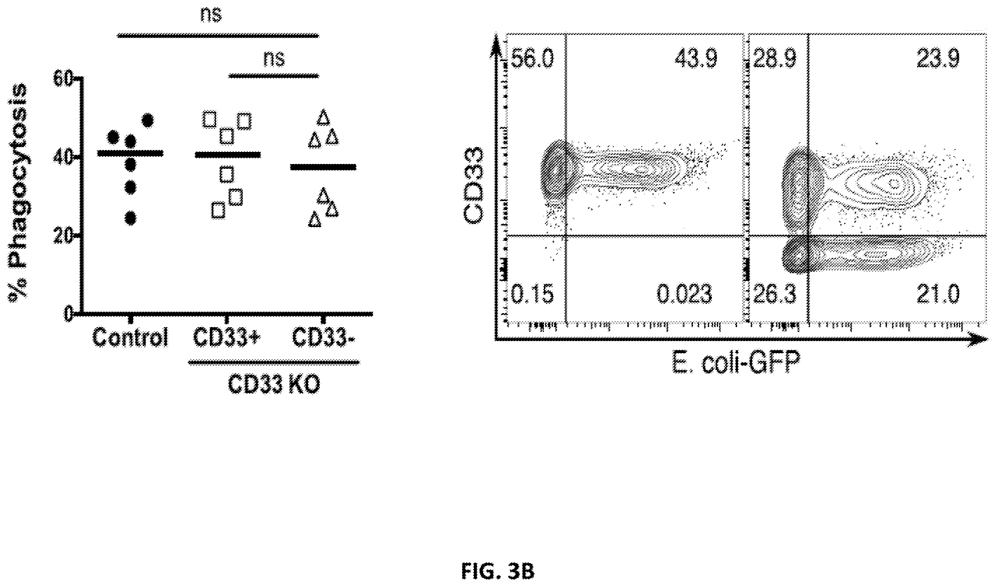

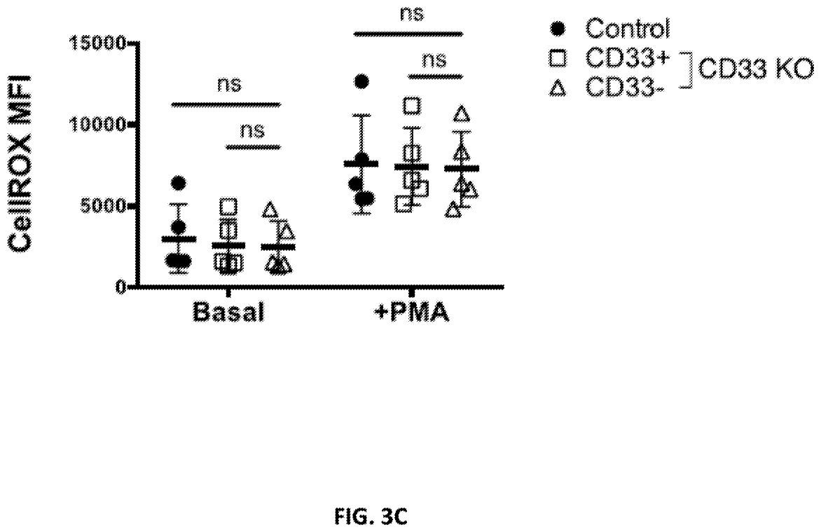

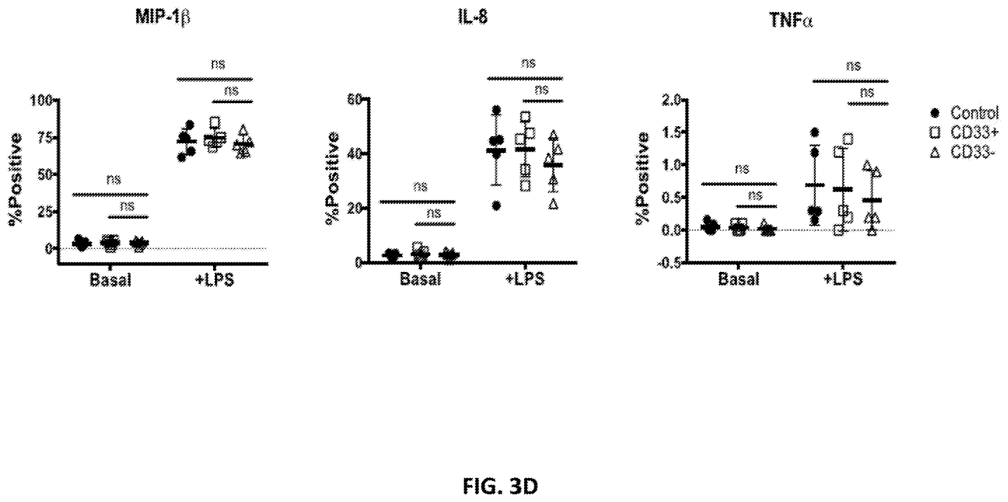

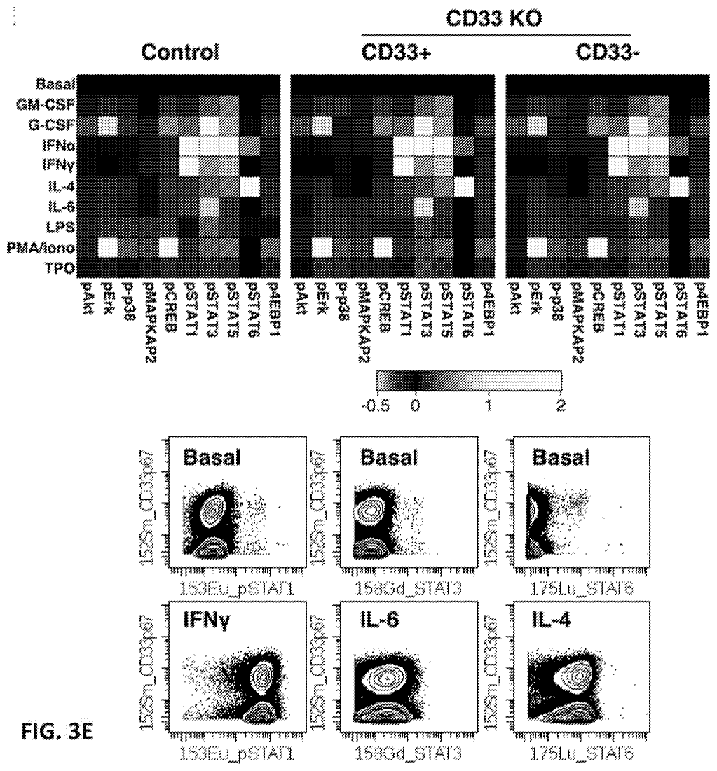

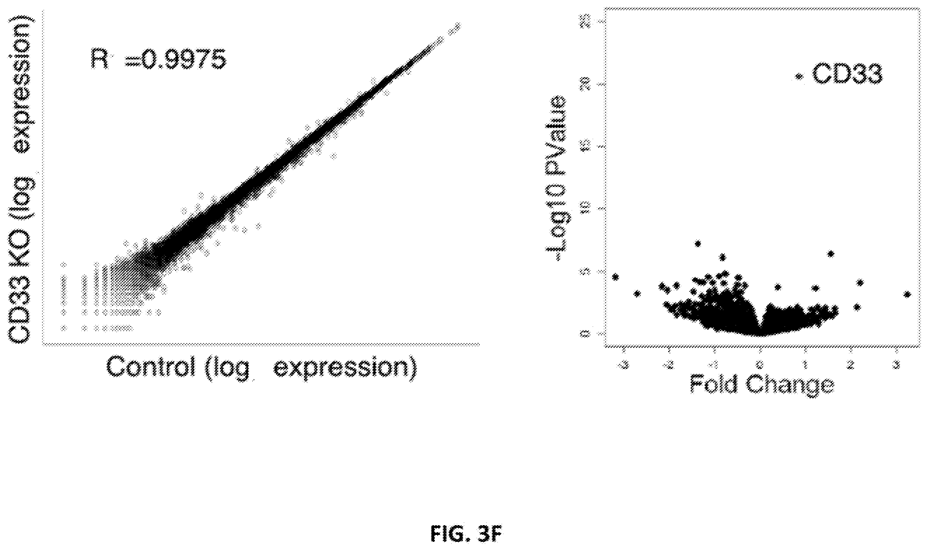

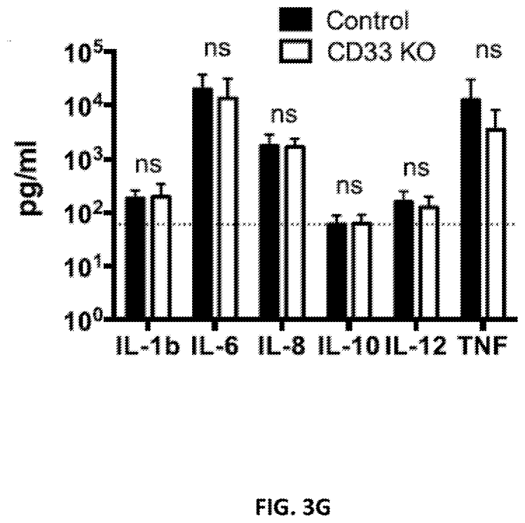

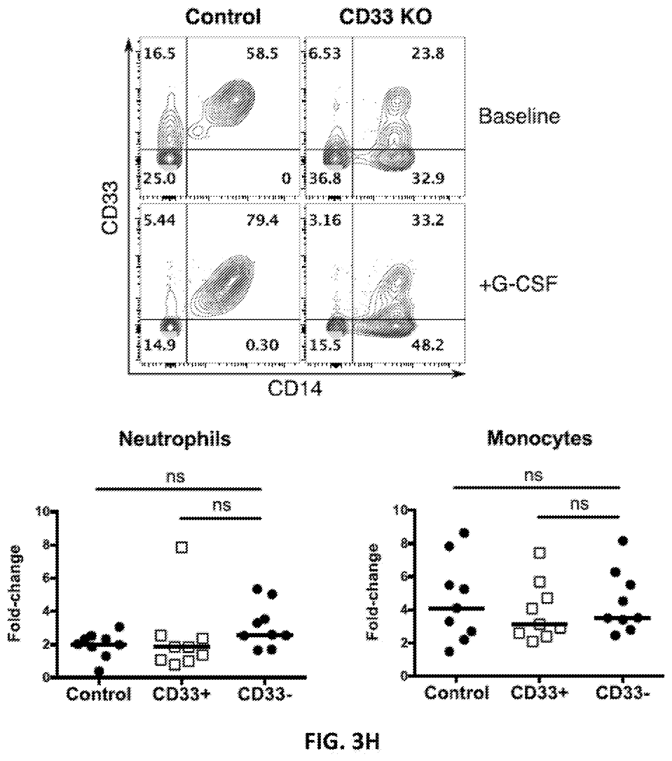

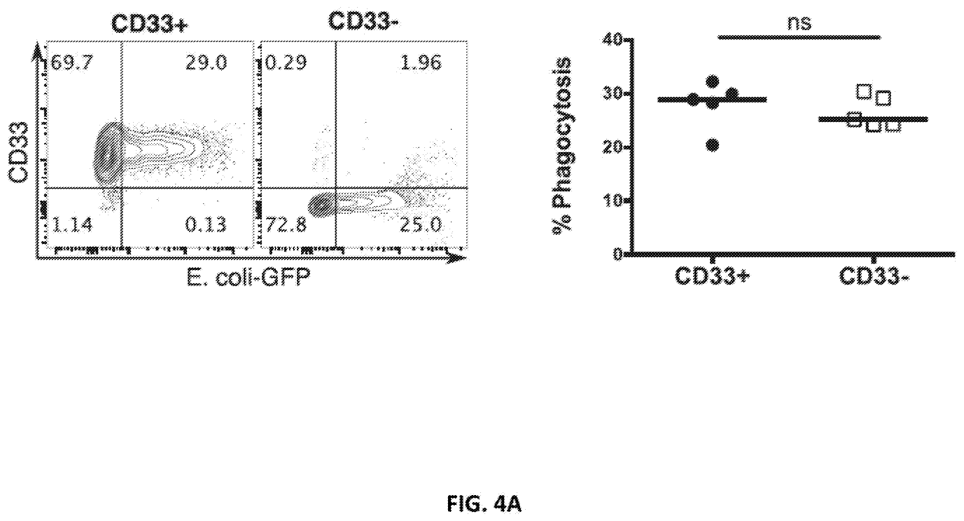

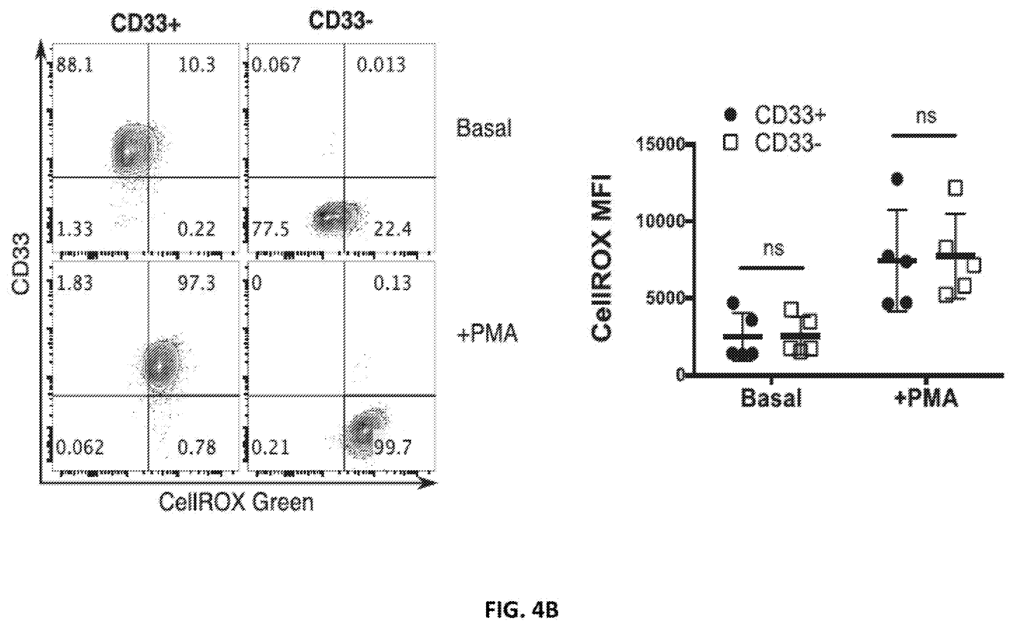

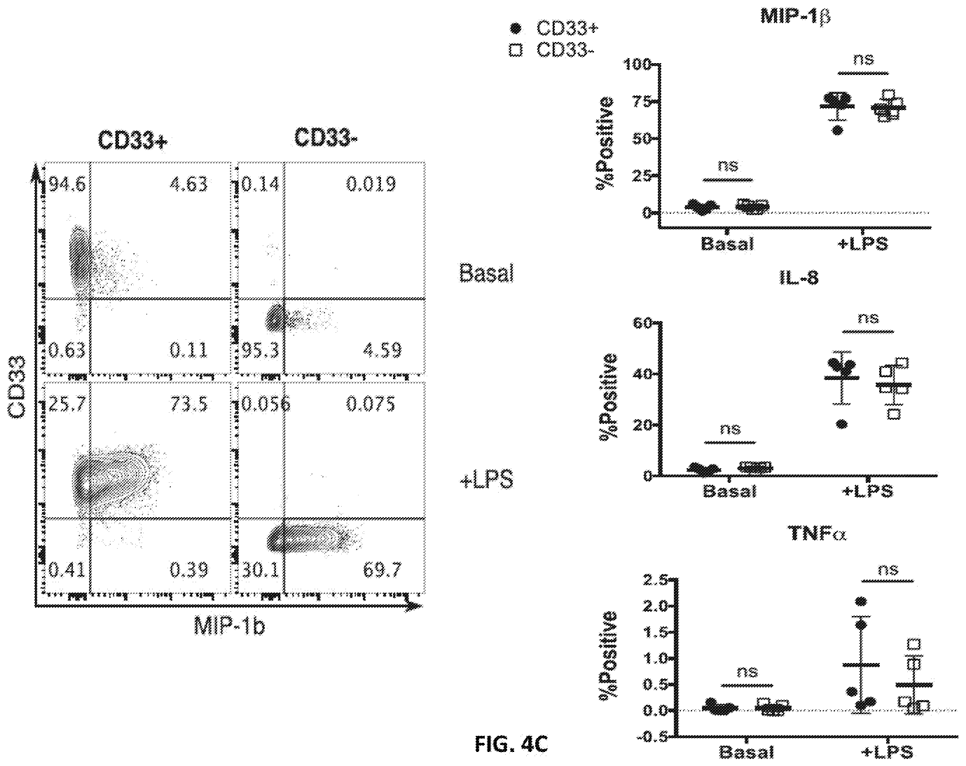

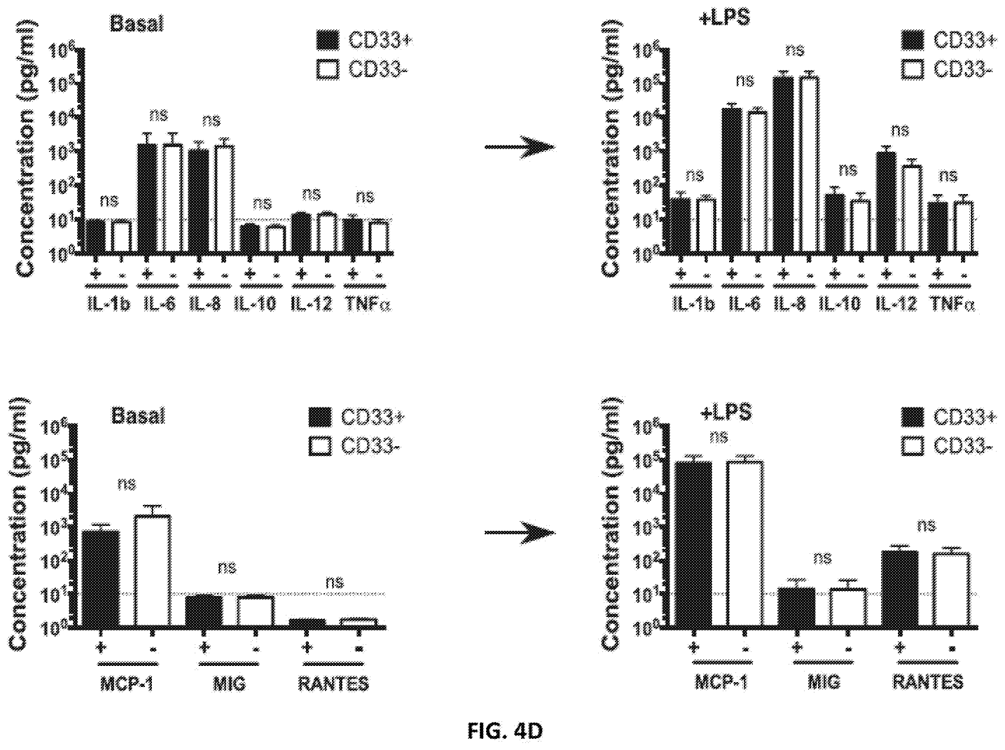

Several studies were performed to probe the function of CD33 KO myeloid cells. Human cells in the marrow of mice engrafted with CD33 KO HSPCs were morphologically normal, and absence of CD33 was confirmed by immunocytochemistry (FIG. 3A). The ability of in vitro differentiated CD33 KO myeloid cells to perform phagocytosis, generate reactive oxygen species, and secrete inflammatory cytokines/chemokines was interrogated (FIGS. 3B-3D). HSPCs that had approximately 50% CD33 KO were used and the CD33+ and CD33− cells were gated on separately. CD33+ cells were used as an internal control while also compared to control HSPCs. These studies were also performed on cells sorted for CD33+/− expression to exclude any non-cell autonomous function of CD33 (FIGS. 4A-4D). No significant difference was found in any of the studies when comparing the function of CD33− myeloid cells to control cells that were uniformly CD33+.

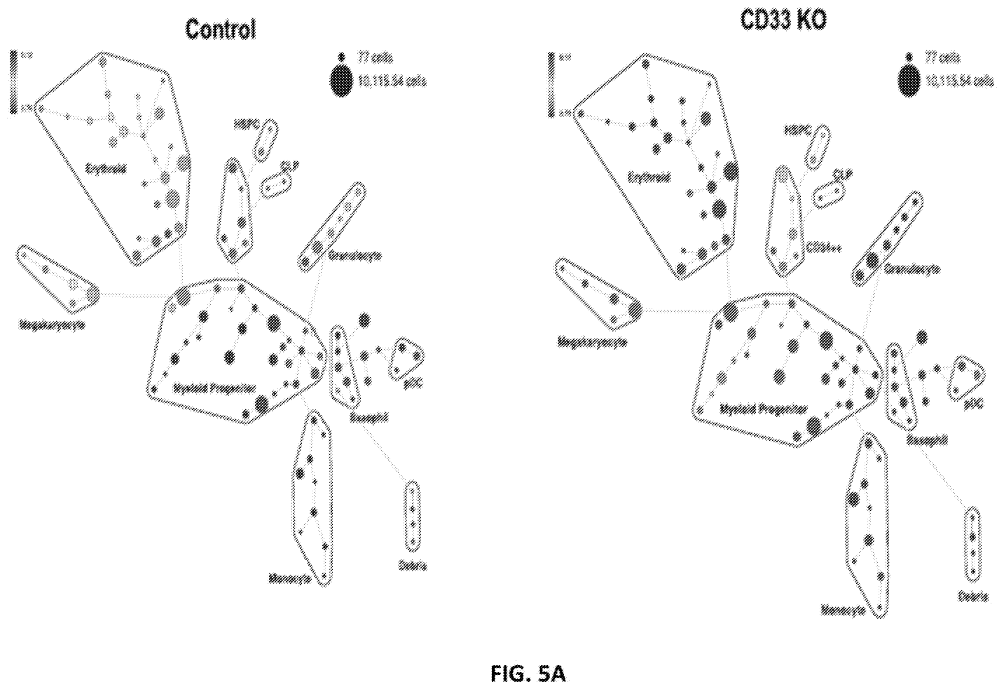

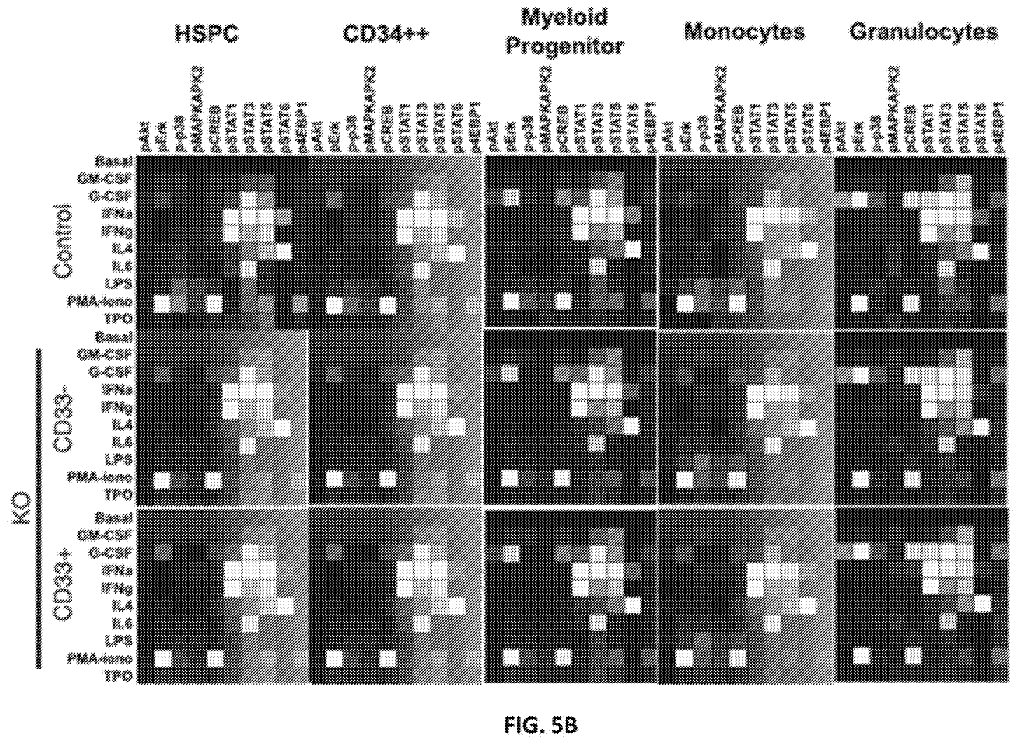

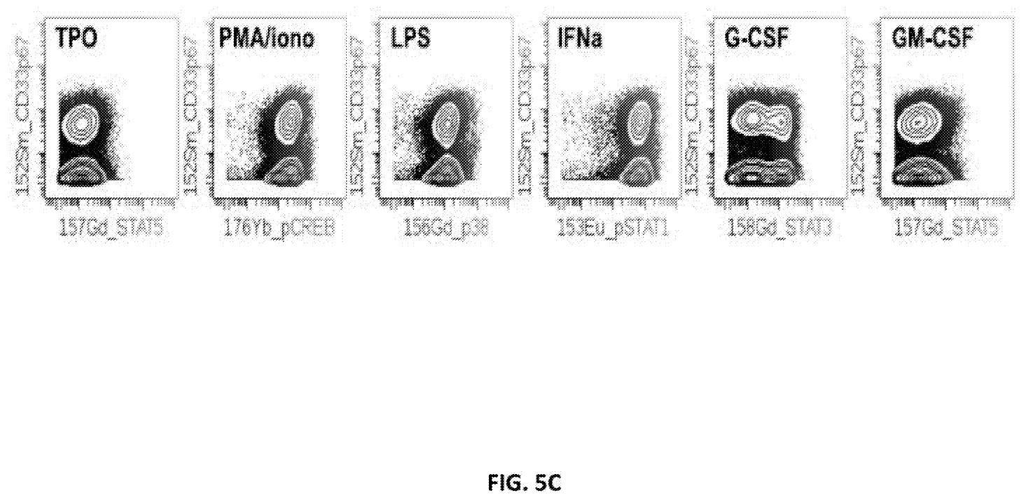

High-dimensional functional profiling of control and CD33 KO HSPCs was performed using mass cytometry. Other than the expected differences in CD33 expression, no differences were found in the differentiation profile between control and CD33 KO cells by SPADE analysis (FIG. 5A). Cells were exposed to relevant ex vivo perturbations (granulocyte-macrophage colony stimulator factor [GM-CSF], G-CSF, interferon [IFN]a, IFNg, interleukin [IL]-4, IL-6, lipopolysaccharide [LPS], phorbol myristate acetate [PMA]/ionomycin, thrombopoietin [TPO]) and activation of intracellular signaling pathways in relevant hematopoietic subpopulations was quantified. Remarkably, control and CD33 KO cells responded identically to external stimuli, and within the CD33 KO cell population, both the CD33+ and CD33− cells responded to the same degree (FIGS. 3E and 5B-5C).

To exclude a major impact of CD33 loss on downstream gene expression, RNA sequencing (RNA-seq) of CD33 KO myeloid cells and controls was performed, which showed a highly concordant gene expression profile. Paired analysis of matched control and CD33 KO samples showed that CD33 was the most significantly differentially expressed gene (FIG. 3F).

Studies in mice engrafted with CD33 KO or control human hematopoiesis were performed to evaluate their functional properties in vivo. To model gram-negative sepsis, mice were injected with lipopolysaccharide, which induced high levels of human inflammatory cytokines in the serum with no difference detected between control and CD33 KO (FIG. 3G). The mice were also challenged with G-CSF, which induced an equivalent increase of human neutrophils (CD66b+) and monocytes (CD14+) in the peripheral circulation, while in the CD33 KO HSPC-engrafted mice, both CD33+ and CD33− myeloid cells increased to the same degree (FIG. 3H). These observations confirmed the hypothesis that human myeloid cells would tolerate loss of CD33.

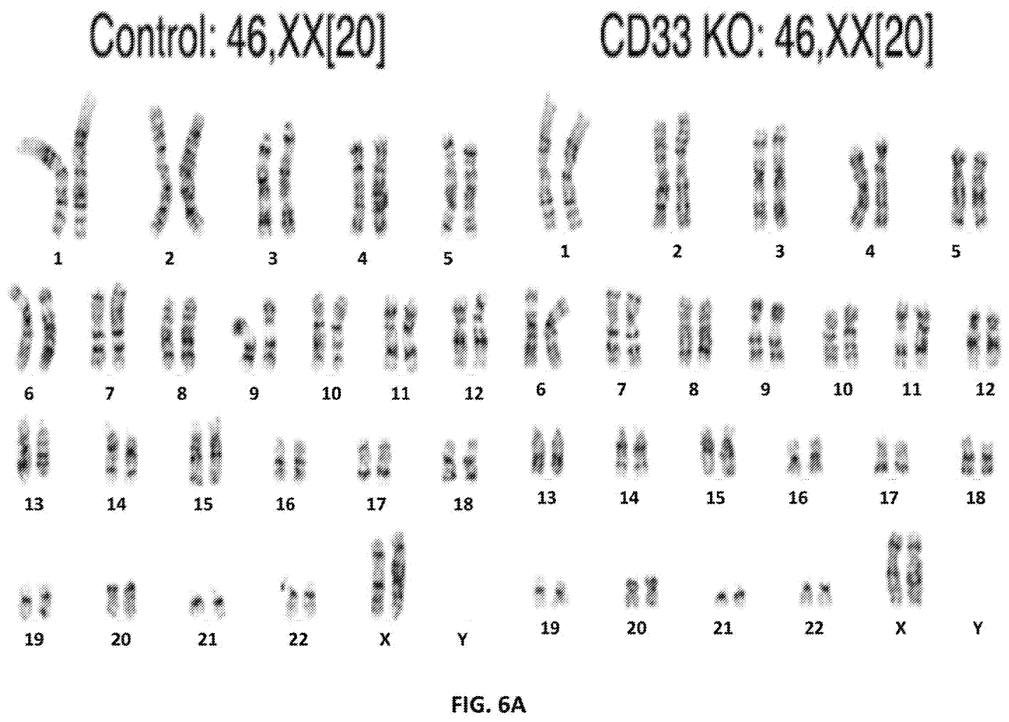

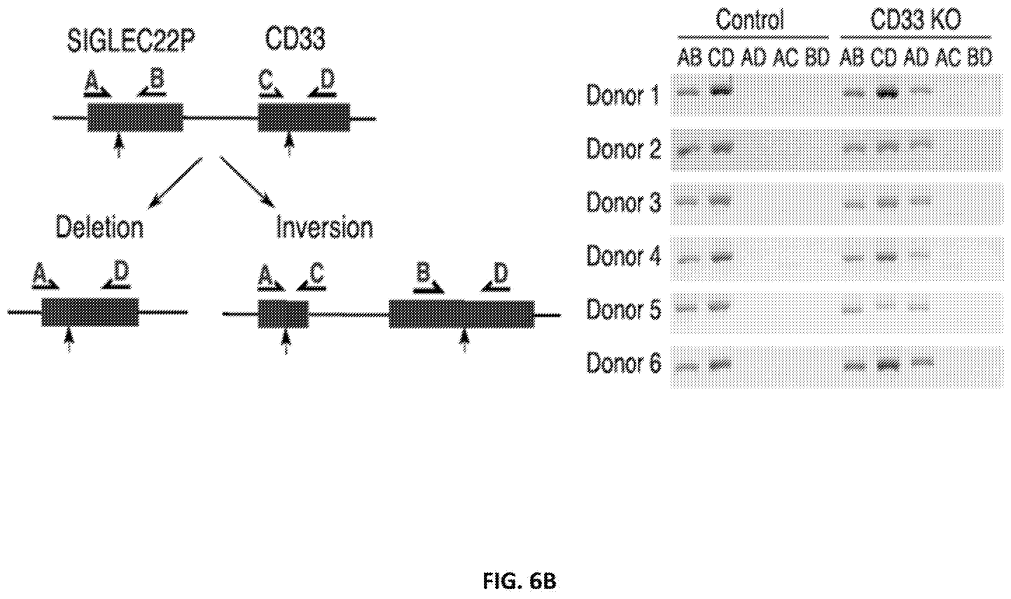

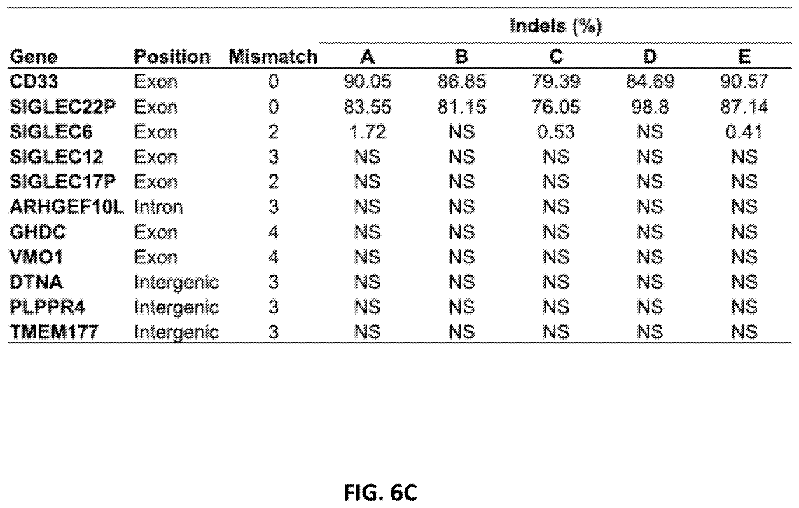

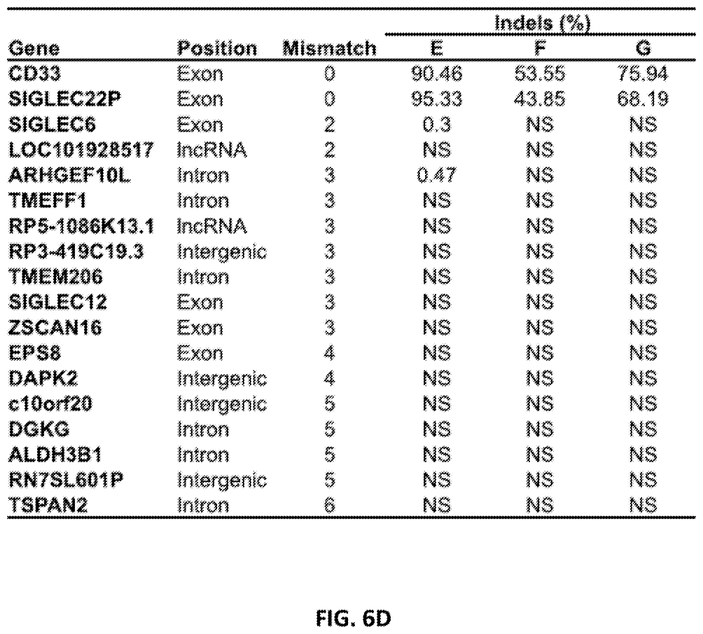

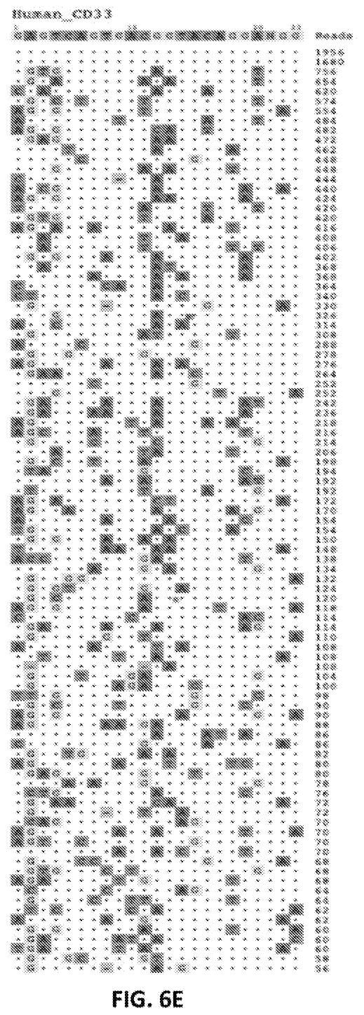

For additional evidence that CD33 may be redundant in human health, several databases of naturally occurring homozygous loss-of-function mutations in large population cohorts were examined (Lek et al., (2016) Nature 536, 285-291; Narasimhan et al., (2016) Science 352, 474-477; Saleheen et al., (2017) Nature 544, 235-239; Sulem et al., (2015) Nat. Genet. 47, 448-452). Fifty-one individuals were reported to have homozygous loss-of-function mutations in the CD33 gene, the majority (86%) of which consisted of a deletion of four base pairs in exon 3 (rs201074739), resulting in a frameshift mutation in all protein-coding transcripts of CD33. In the Iceland cohort (Sulem et al., (2015) Nat. Genet. 47, 448-452), four of these individuals were reported to have reached 64 to 93 years of age. To evaluate whether the CRISPR/Cas9-based CD33 KO system would cause any off-target mutagenesis, cytogenetic analysis was performed on multiple control/CD33 KO primary samples for gross chromosomal rearrangements. No off target events were detected (FIG. 6A). Six donor samples were further analyzed by PCR and all showed evidence of deletion of the 14-kb fragment between CD33 and SIGLEC22P, a pseudogene that has 100% homology with CD33 at the gRNA binding site (FIG. 6B). These two genes are directly adjacent to each other, with no known genetic element in the intervening sequence. Targeted deep sequencing of the top 10 predicted off-target sites in primary CD34+ cells after CD33 KO was performed and compared with control cells from the same donor (FIG. 6C). In all samples, the on-target site, CD33, had high levels of mutations (86%±4%), and SIGLEC22P harbored a similar extent of mutations as the target gene (85%±9%). Only one additional site, SIGLEC6, was found to have low-frequency mutations (0.41%-1.72%) in three of the five donors evaluated. As a more comprehensive screen for all potential off-target mutation sites in a genome-wide unbiased manner, circularization for in vitro reporting of cleavage effects by sequencing (CIRCLE-seq) was performed on genomic DNA from human primary cells and 95 sites cleaved by the Cas9/CD33-gRNA complex in vitro were identified (FIGS. 6D-6E). Targeted deep sequencing was then performed on primary CD34+ cells in 16 sites that had a high read count on CIRCLEseq and/or had a three-or-less nucleotide mismatch to the on target sequence. Most of the sites had no detectable mutations in primary cells.

### Example 2: CD33 KO HSPCs are Resistant to CD33-Targeted Therapy

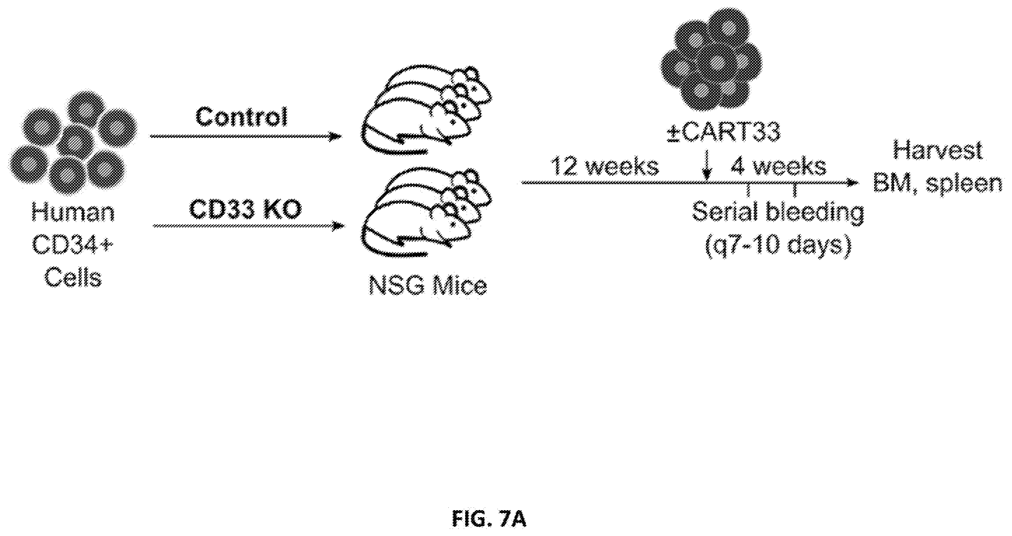

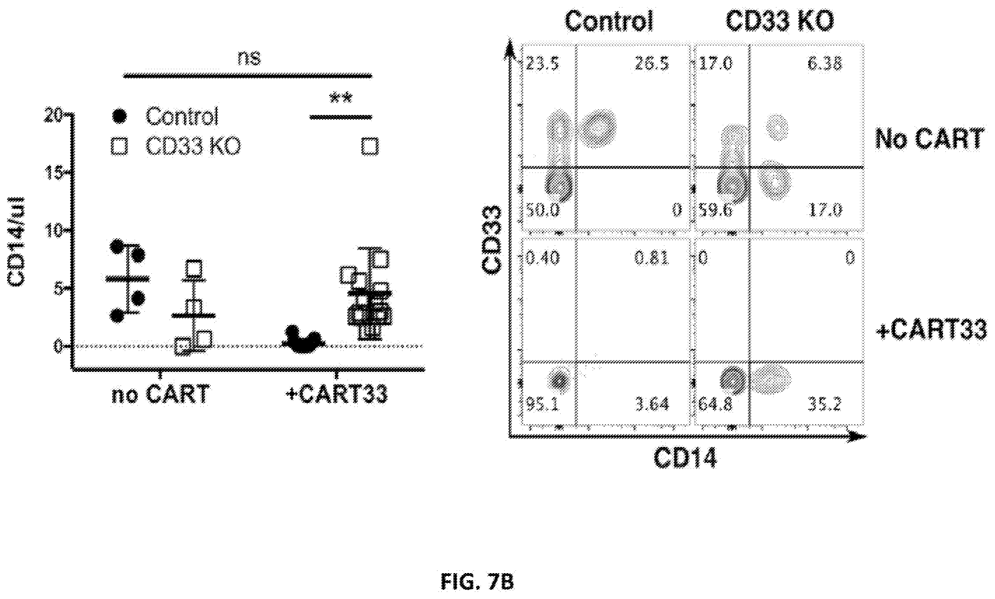

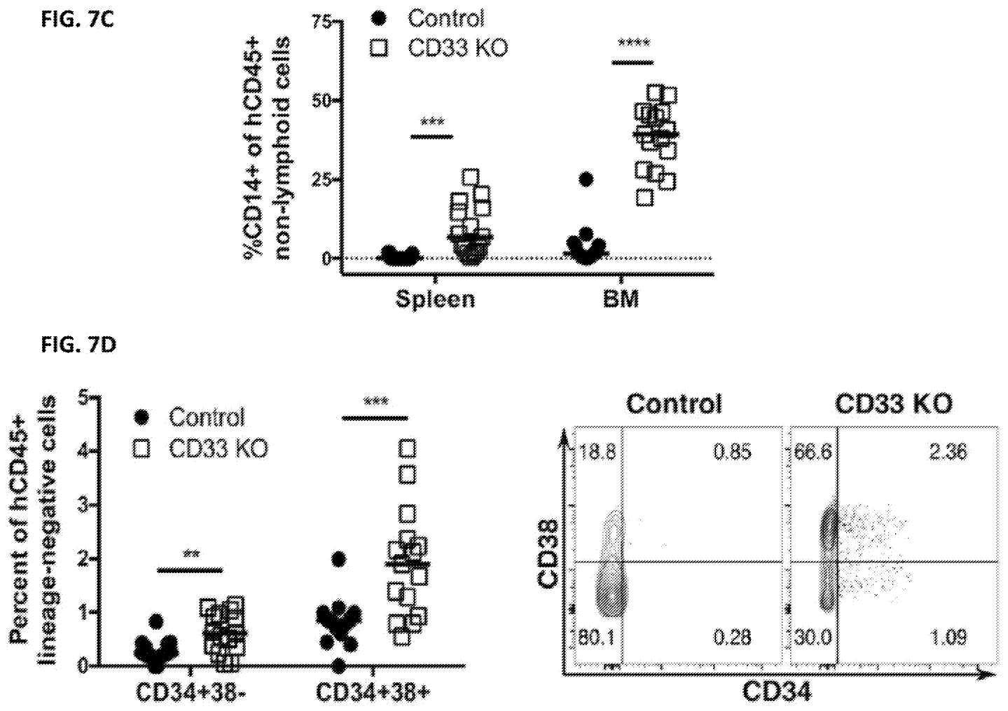

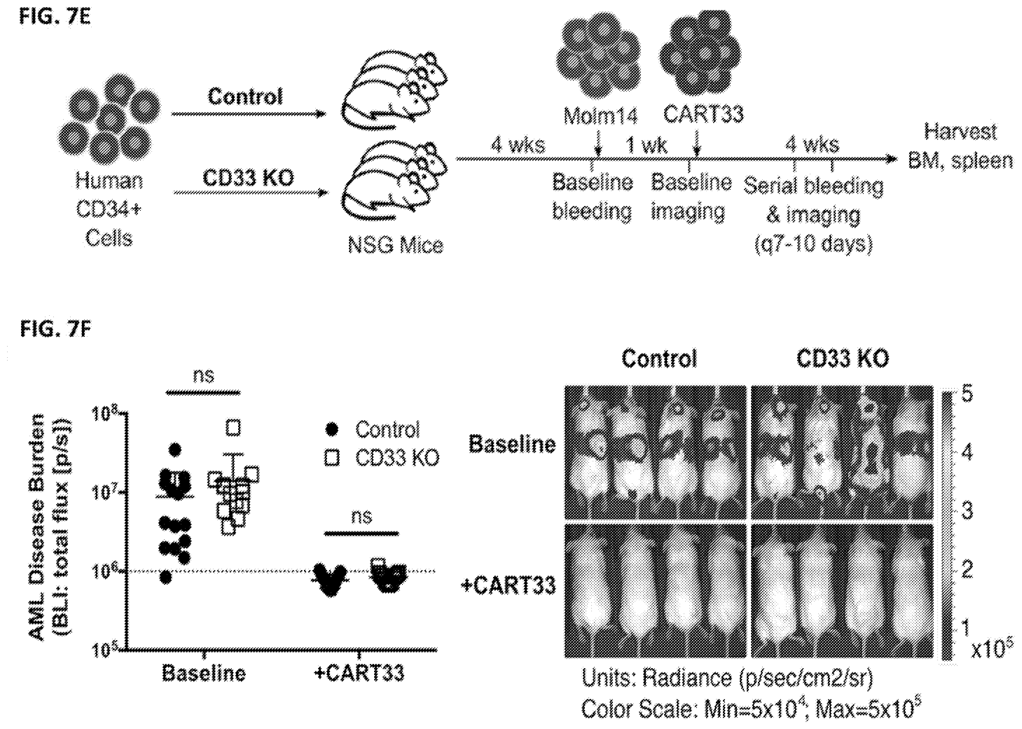

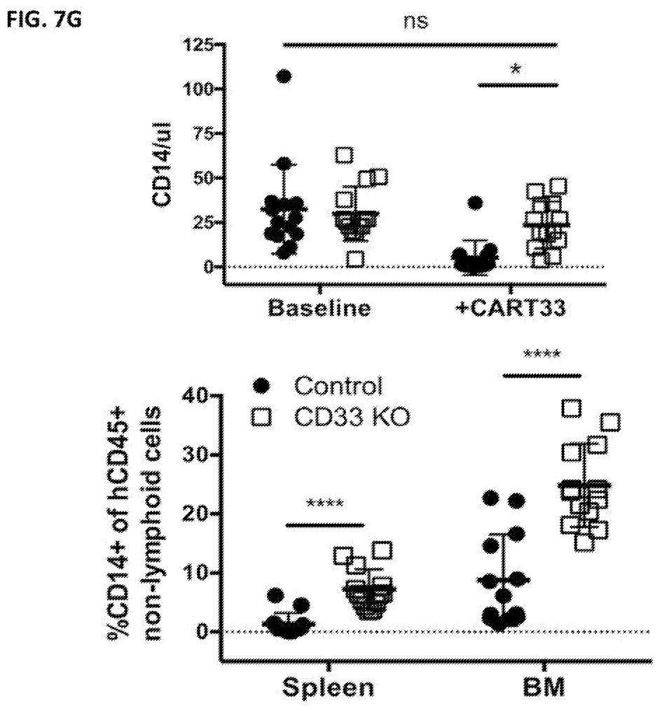

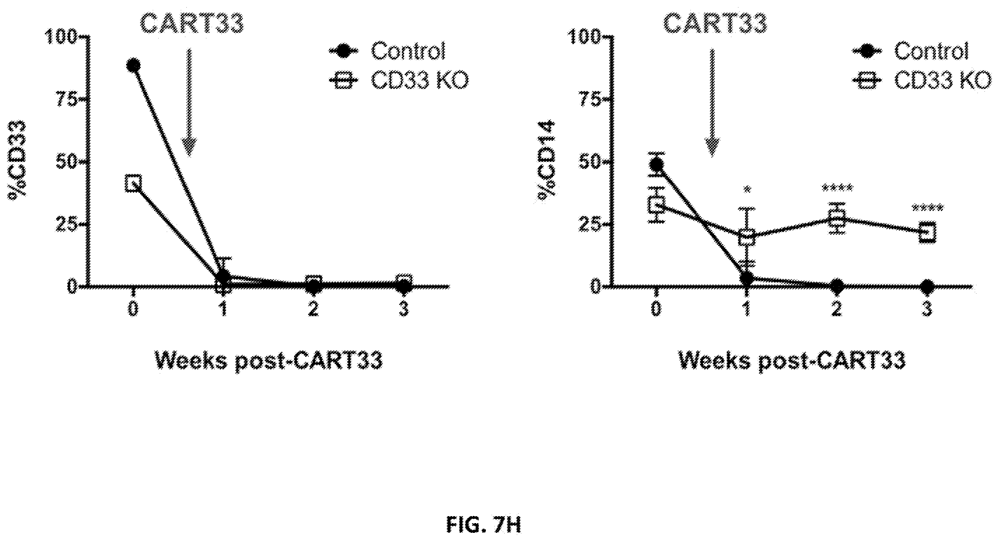

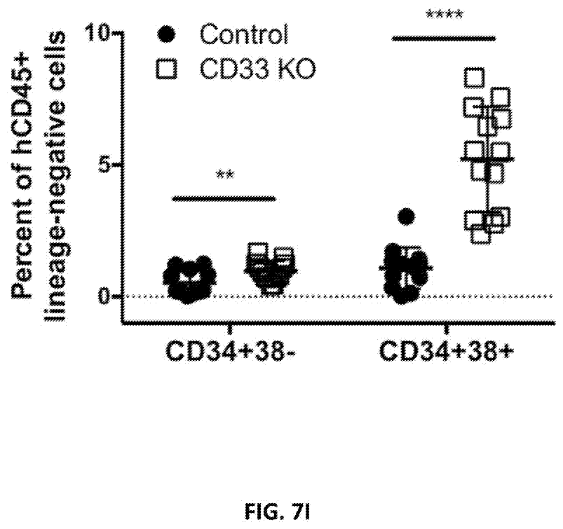

To demonstrate resistance of CD33 KO HSPCs to CD33-targeted therapy, human xenograft models were used. CART cells targeting CD33 (CART33) have been shown to eradicate AML while also eliminating normal CD33+ myeloid cells. Mice engrafted with control or CD33 KO HSPCs were treated with autologous T cells transduced with lentivirus encoding an anti-CD33-41BB-CD3z CAR as previously reported (Kenderian et al., (2015) Leukemia 29, 1637-1647) (FIG. 7A). All CD33+ cells were eliminated in both groups, leading to complete disappearance of CD14+ human monocytes in the control mice (FIG. 7B). Crucially, in the mice engrafted with CD33 KO HSPCs, CD14+ cells were retained in the peripheral blood, spleen, and marrow (FIGS. 7B-7C). Sparing of human hematopoietic stem cells (CD34+38−) and progenitors (CD34+38+) were also found in the marrow of mice engrafted with CD33 KO HSPCs compared to controls, showing that the CD33 KO HSPCs and their myeloid progeny are indeed resistant to CD33-targeted therapy (FIG. 7D).

The presence of CD33+ AML has the potential to stimulate CART33 proliferation and activation and thus cause bystander myelotoxicity. Therefore, the resistance of CD33 KO HSPCs was further interrogated by injecting mice engrafted with control or CD33 KO HSPCs with Molm14, a CD33+ AML cell line. Engraftment of Molm14 was confirmed by bioluminescent imaging and then the mice were treated with CART33 (FIG. 7E). Leukemia responded to CART33 treatment in all mice (FIG. 7F). Importantly, rapid clearance of CD33+ cells leading to myeloablation of non-leukemic human cells in the control HSPC mice was again found, while mice engrafted with CD33 KO HSPCs continued to sustain differentiated human myeloid cells and HSPCs despite clearance of leukemic cells (FIGS. 7G-7I). These studies showed that CD33 KO HSPCs can be used to circumvent the myeloid toxicity of potent CD33-targeted therapy while still permitting persistent anti-tumor efficacy.

### Example 3: Optimization of CRISPR/Cas9 Knock-Out of CD33 Human Hematopoietic Stem/Progenitor Cells (HSPCs) for Allogenic Transplantation in Adult Patients with Relapsed/Refractory Acute Myeloid Leukemia (AML)

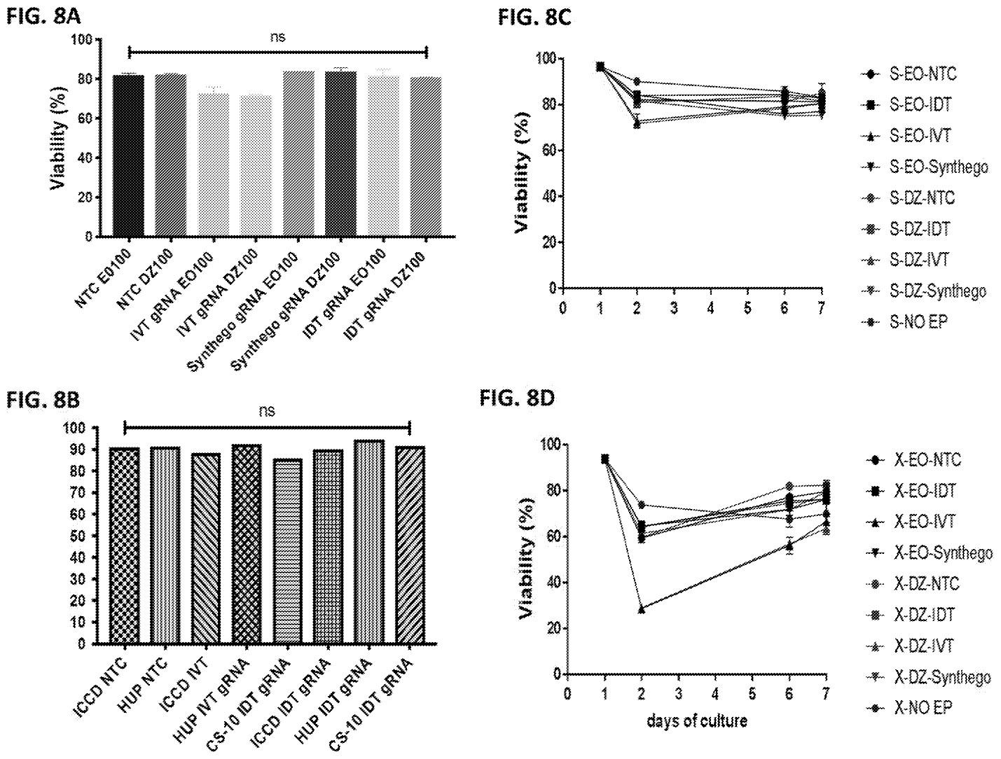

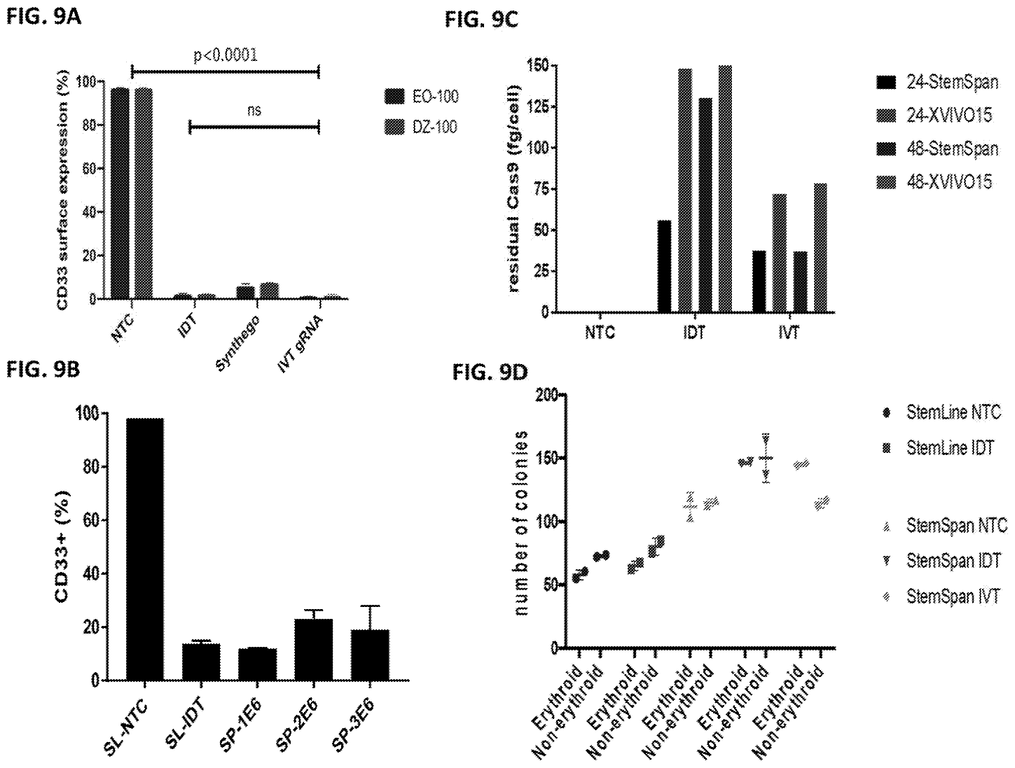

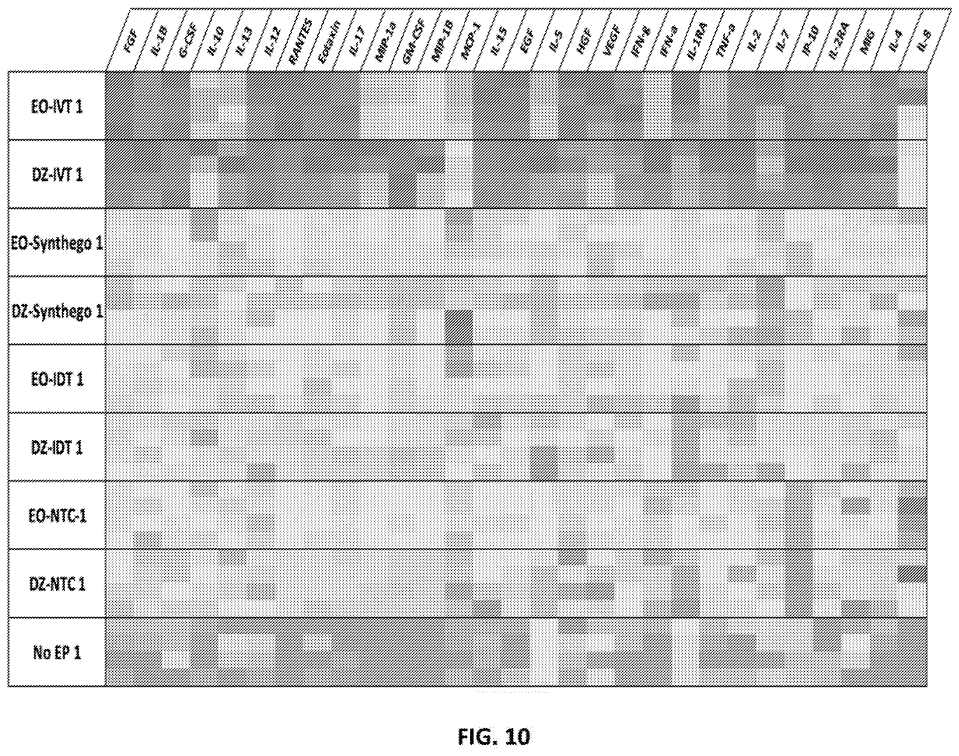

Human CD34+ cells were isolated from frozen G-CSF mobilized peripheral blood using Miltenyi Biotech CD34 human microbeads. For experiments described herein, the Lonza 4-D device was used at small and medium-scale (20 uL or 100 uL cuvettes, respectively). Two different pulse codes evaluated showed similar KO efficiency though with slightly improved cell viability with EO-100 (FIGS. 8A & 9A). In vitro transcribed (IVT) CD33-targeting guide RNA (gRNA) was compared with synthetic modified CD33-targeting gRNA manufactured by two different vendors (Synthego and Integrated DNA Technologies, IDT). IDT gRNA showed better KO efficiency compared to Synthego gRNA (FIG. 9A). IVT gRNA had higher KO efficiency compared with both synthetic gRNAs (FIG. 9A) however cell viability was improved with synthetic gRNA (FIG. 8A) and caused less cytokine perturbation (FIG. 10).

Three different media for liquid culture were evaluated, with better cell viability seen with StemSpan compared with X-Vivo (FIGS. 9C-9D) and higher colony forming units with StemSpan compared with StemLine (FIG. 9D). Residual Cas9 protein was detected by ELISA and levels were lower when IVT gRNA was used compared with IDT gRNA (FIG. 9C). Cells were frozen in three different media: ICCD (CVPF IC+CD freeze media=7.5% DMSO), HUP (7.5% DMSO), and CS-10 (10% DMSO). No difference was seen in post thaw viability of electroporated cells between the three different freeze media (FIG. 8B).

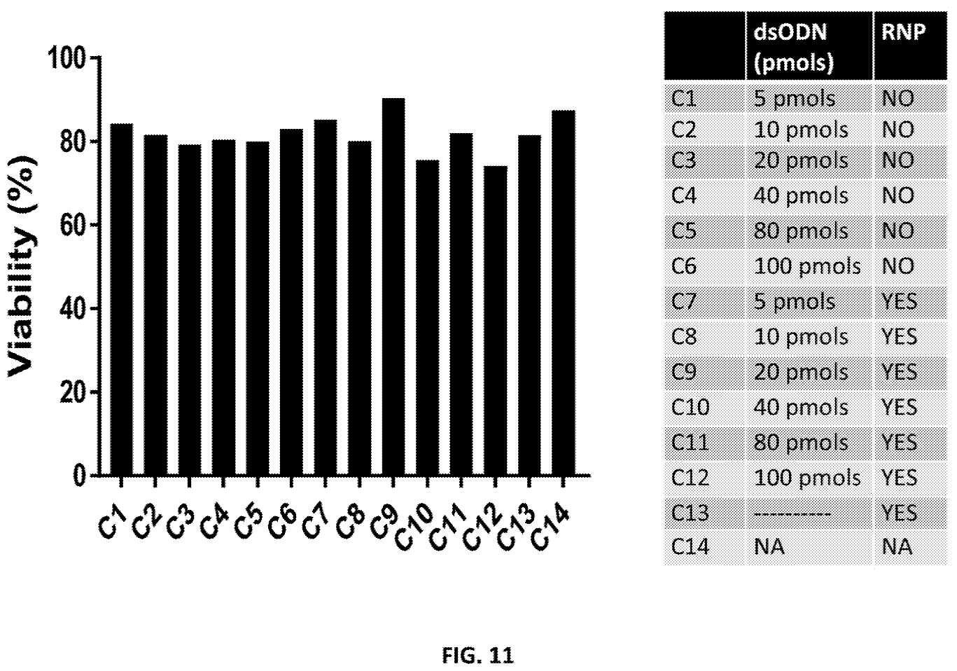

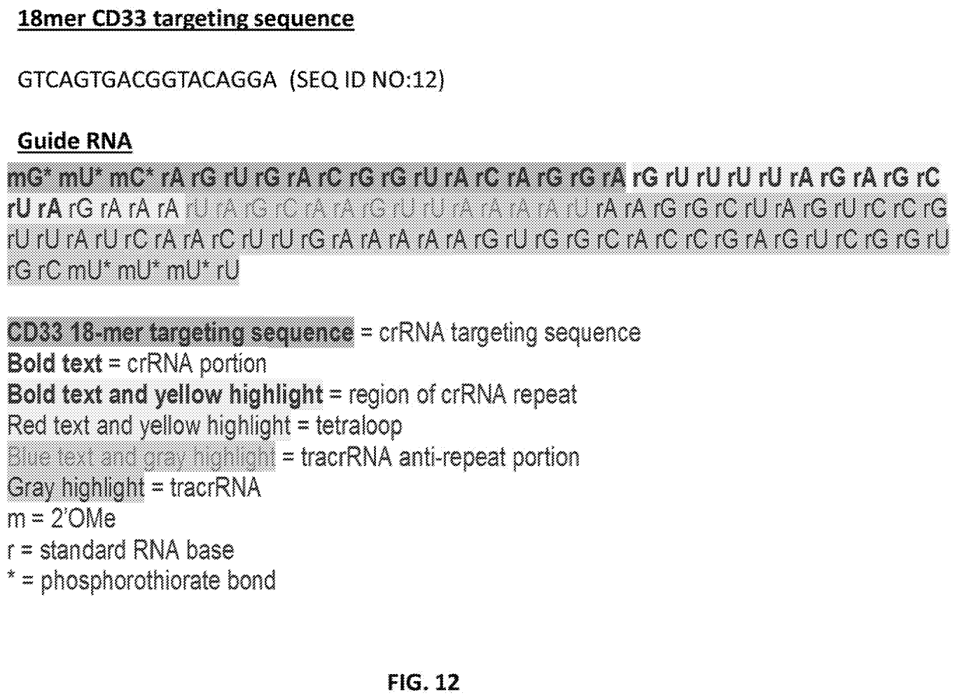

A double stranded oligodeoxynucleotide (dsODN) was generated and titrated from 100 pmols-5 pmols (FIG. 11). Cells were incubated with the various concentrations of dsODN, with or without RNP, and cell viablility measured. RNP− cells were included to detect background double stranded break frequency not related to CRISPR/Cas9 activity. No difference in cell viability was observed among the various concentrations of dsODN (FIG. 11).

Herein was optimized a GMP-compliant system for CRISPR-based editing of human HSPC that is poised for translation to clinical scale.

### Example 4: On-Target Efficiency and Off-Target Risk

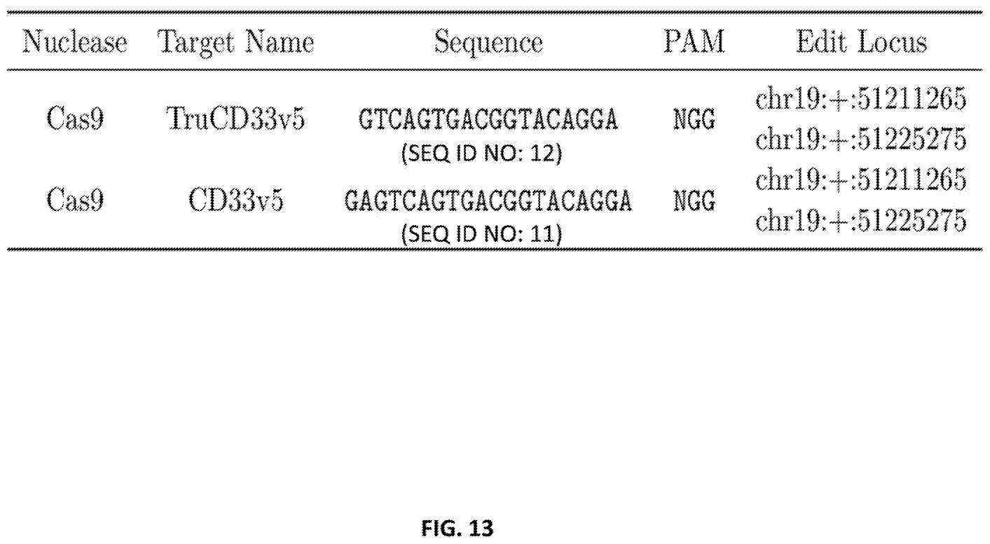

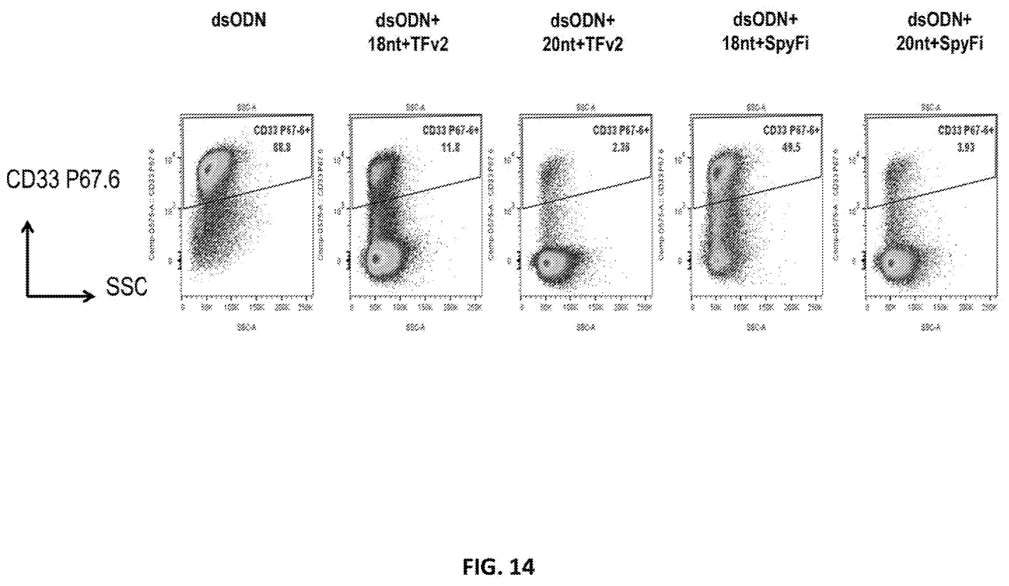

Two chemically modified gRNAs, differing by two nucleotides at the 5′ end of the targeting sequence (FIG. 13), were compared. The 18nt and 20 nucleotide gRNAs were combined with a dsODN and either ThermoFisher® version 2 Cas9 (TFv2) or SpyFi™ Cas9 from Aldevron® (FIGS. 14-16). On-target efficiency was measured by flow cytometry. Results showed that TFv2 Cas9 with 20nt gRNA and SpyFi™ Cas9 with 20nt gRNA were the most efficient, followed by TFv2 Cas9 with 18nt gRNA, and then SpyFi™ Cas9 with 18nt gRNA (FIG. 14). On-target efficiency and off-target risk were also analyzed by GUIDEseq. TFv2 Cas9 with 20nt gRNA and SpyFi™ Cas9 with 20nt gRNA demonstrated the best on-target efficiency, followed by TFv2 Cas9 with 18nt gRNA, and then SpyFi™ Cas9 with 18nt gRNA.

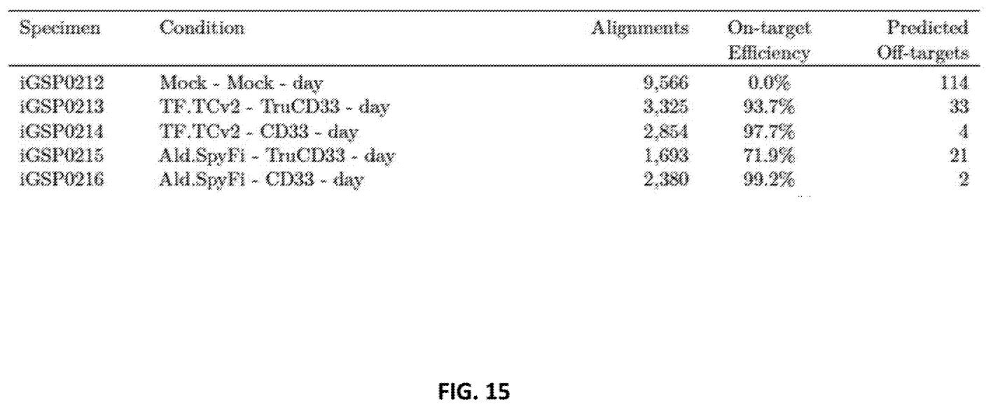

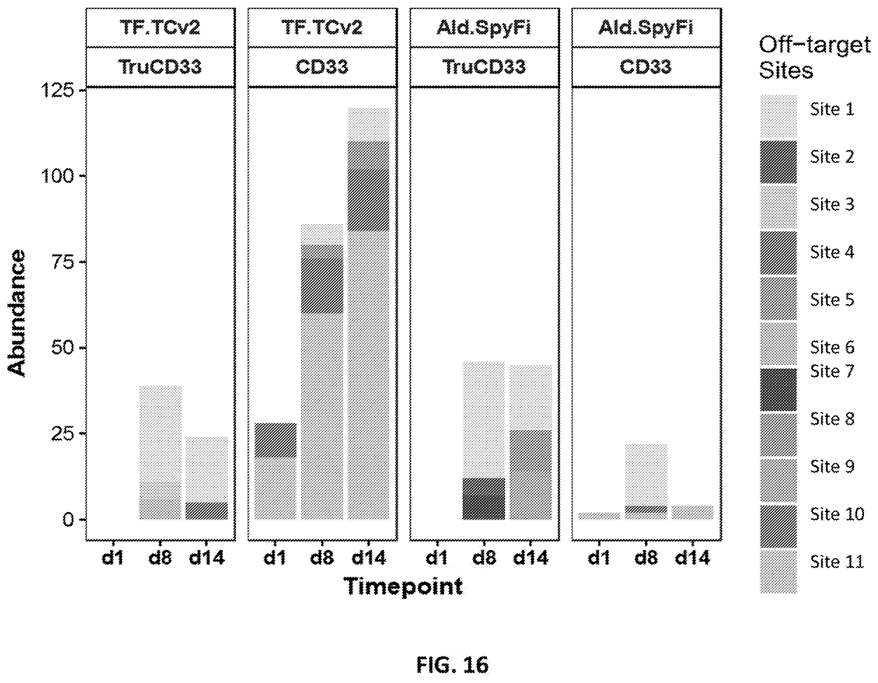

SpyFi™ Cas9 with 20nt gRNA had the lowest number of predicted off-target events followed by TFv2 Cas9 with 20nt gRNA, SpyFi™ Cas9 with 18nt gRNA, and then TFv2 Cas9 with 18nt gRNA (FIG. 15). Off-target detection (with correction) also demonstrated that the combination of SpyFi™ Cas9 with 20nt gRNA generated the least number of off-target sites (FIG. 16).

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A chemically modified CD33-targeting guide RNA comprising:
a crRNA portion comprising (i) a guide sequence capable of hybridizing to a target endogenous gene locus encoding for CD33, and (ii) a repeat sequence; and
a tracrRNA portion comprising an anti-repeat sequence that is complementary to the repeat sequence,
wherein the crRNA portion and/or the tracrRNA portion comprise(s) one or more modified nucleotides.

2. The chemically modified guide RNA of claim 1, wherein
(a) the crRNA portion comprises one or more modified nucleotides, and/or
(b) the tracrRNA portion comprises one or more modified nucleotides, and/or
(c) the crRNA portion and the tracrRNA portion independently comprise one or more modified nucleotides, and/or
(d) the one or more modified nucleotides independently comprise a modification of a ribose group, a phosphate group, or any combinations thereof and/or
(e) the one or more modified nucleotides independently comprise a modification of a ribose group selected from the group consisting of 2′-O-methyl, 2′-fluoro, 2′-deoxy, 2′-O-(2-methoxyethyl) (MOE), 2′-NH₂, or a bicyclic nucleotide such as locked nucleic acid (LNA), 2′-(S)-constrained ethyl (S-cEt), constrained MOE, and 2′-O,4′-C-aminomethylene bridged nucleic acid (2′,4′-BNA^{N}C), and/or
(f) the one or more modified nucleotides independently comprise a modification of a phosphate group selected from the group consisting of a phosphorothioate, phosphonoacetate (PACE), thiophosphonoacetate (thioPACE), amide, triazole, phosphonate, or phosphotriester modification
(g) nucleotides at positions 1-3 from the 5′ end of the crRNA portion comprise 2′-O-methyl modifications and/or
(h) nucleotides at positions 2-4 from the 3′ end of the tracrRNA portion comprise 2′-O-methyl modifications and/or
(i) nucleotides at positions 1-4 from the 5′ end of the crRNA portion comprise phosphorothioate modifications and/or
(j) nucleotides at positions 1-4 from the 3′ end of the tracrRNA portion comprise phosphorothioate modifications and/or
(k) the guide sequence comprises the nucleic acid sequence GUCAGUGACGGUACAGGA (SEQ ID NO:1) and/or
(l) the guide sequence consists of the nucleic acid sequence GUCAGUGACGGUACAGGA (SEQ ID NO:1) and/or
(m) the guide sequence comprises the nucleic acid sequence mG*mU*mC*AGUGACGGUACAGGA (SEQ ID NO: 2),
wherein m indicates a 2′-O-methyl modification, and
wherein * indicates a phosphorothioate modification, and/or
(n) the guide sequence consists of the nucleic acid sequence mG*mU*mC*AGUGACGGUACAGGA (SEQ ID NO: 2),
wherein m indicates a 2′-O-methyl modification, and
wherein * indicates a phosphorothioate modification, and/or
(o) the guide sequence comprises the nucleic acid sequence GAGUCAGUGACGGUACAGGA (SEQ ID NO:16), and/or
(p) the guide sequence consists of the nucleic acid sequence GAGUCAGUGACGGUACAGGA (SEQ ID NO:16), and/or
(q) the guide sequence comprises the nucleic acid sequence mG*mA*mG*UCAGUGACGGUACAGGA (SEQ ID NO: 17),
wherein m indicates a 2′-O-methyl modification, and
wherein * indicates a phosphorothioate modification, and/or
(r) the guide sequence consists of the nucleic acid sequence mG*mA*mG*UCAGUGACGGUACAGGA (SEQ ID NO: 17),
wherein m indicates a 2′-O-methyl modification, and
wherein * indicates a phosphorothioate modification.

3.-20. (canceled)

21. A CD33-targeting guide RNA comprising:
a crRNA portion comprising (i) a guide sequence consisting of the nucleic acid sequence GUCAGUGACGGUACAGGA (SEQ ID NO:1) or GAGUCAGUGACGGUACAGGA (SEQ ID NO:16), capable of hybridizing to a target endogenous gene locus encoding for CD33, and (ii) a repeat sequence; and
a tracrRNA portion comprising an anti-repeat sequence that is complementary to the repeat sequence.

22.-23. (canceled)

24. The guide RNA of claim 1, wherein
(a) the guide RNA is in a complex with a Cas9 nuclease, and/or
(b) the Cas9 nuclease is an S. pyogenes Cas9 nuclease or an S. aureus Cas9 nuclease, and/or
(c) the Cas9 nuclease is a variant S. pyogenes Cas9 nuclease, and/or
(d) the variant Cas9 nuclease comprises reduced off-target activity, and maintained or increased on-target activity relative to a wild-type Cas9, and/or
(e) the variant Cas9 nuclease is a SpyFi™ Cas9 nuclease.

25.-28. (canceled)

29. A method for generating a population of modified hematopoietic stem and progenitor cells (HSPCs), the method comprising introducing into the HSPCs the CD33-targeting guide RNA of claim 1, wherein the chemically modified guide RNA produces an insertion and/or deletion capable of downregulating gene expression of CD33.

30. The method of claim 29, wherein
(a) the HSPCs are resistant to a CD33-targeted cell therapy and/or a CD33 CAR-T therapy, and/or
(b) the HSPCs are autologous cells obtained from a source selected from the group consisting of peripheral blood mononuclear cells, cord blood cells, bone marrow, lymph node, and spleen, and/or
(c) the HSPCs are CD34+ HSPCs, and/or
(d) the guide sequence comprises the nucleic acid sequence GUCAGUGACGGUACAGGA (SEQ ID NO:1), and/or
(e) the guide sequence consists of the nucleic acid sequence GUCAGUGACGGUACAGGA (SEQ ID NO:1), and/or
(f) the guide sequence comprises the nucleic acid sequence GAGUCAGUGACGGUACAGGA (SEQ ID NO:16), and/or
(g) the guide sequence consists of the nucleic acid sequence GAGUCAGUGACGGUACAGGA (SEQ ID NO:16); and/or
(h) further comprising introducing into the HSPCs a homology-directed repair (HDR) template comprising a single-stranded oligonucleotide donor (ssODN) sequence.

31.-40. (canceled)

41. The method of claim 30 (h), wherein
(a) the ssODN sequence comprises (i) the nucleotide sequence GCAGGAGTCAGTGACGGTACAGGAGGGTTTGTGCGTCC (SEQ ID NO: 18) with an extra adenine inserted within the sequence, or (ii) the nucleotide sequence GCAGGAGTCAGTGACGGTACAAGGAGGGTTTGTGCGTCC (SEQ ID NO:3);_or
(b) the ssODN sequence consists of (i) the nucleotide sequence GCAGGAGTCAGTGACGGTACAGGAGGGTTTGTGCGTCC (SEQ ID NO: 18) with an extra adenine inserted within the sequence, or (ii) the nucleotide sequence GCAGGAGTCAGTGACGGTACAAGGAGGGTTTGTGCGTCC (SEQ ID NO:3): or
(c) the ssODN comprises the nucleotide sequence
(SEQ ID NO: 4)
GGTTGTCGGGCTGGGCCGAGCTGACCCTCGTTTCCCCACAGGGGCCCTGG
CTATGGATCCAAATTTCTGGCTGCAAGTGCAGGAGTCAGTGACGGTACAA
GGAGGGTTTGTGCGTCCTCGTGCCCTGCACTTTCTTCCATCCCATACCCT
ACTACGACAAGAACTCCCCAGTTCATGGTTACTGGTTCCGGGAAGGAGC;
or
(d) the ssODN consists of the nucleotide sequence
(SEQ ID NO: 4)
GGTTGTCGGGCTGGGCCGAGCTGACCCTCGTTTCCCCACAGGGGCCCTGG
CTATGGATCCAAATTTCTGGCTGCAAGTGCAGGAGTCAGTGACGGTACAA
GGAGGGTTTGTGCGTCCTCGTGCCCTGCACTTTCTTCCATCCCATACCCT
ACTACGACAAGAACTCCCCAGTTCATGGTTACTGGTTCCGGGAAGGAGC.

42.-52. (canceled)

53. The method of claim 29, wherein
(a) the population of modified HSPCs comprise at least about 20%, 25%, 30%, 35%, 40%, 45%, 50%, 55%, 60%, 65%, 70%, 75%, 80%, 85%, 90%, or 95% modified HSPCs comprising the insertion and/or deletion capable of downregulating gene expression of CD33, and/or
(b) the guide RNA is in a complex with a Cas9 nuclease, and/or
(c) the Cas9 nuclease is an S. pyogenes Cas9 nuclease or an S. aureus Cas9 nuclease, and/or
(d) the Cas9 nuclease is a variant S. pyogenes Cas9 nuclease, and/or
(e) the variant Cas9 nuclease comprises reduced off-target activity, and maintained or increased on-target activity relative to a wild-type Cas9, and/or
(f) the variant Cas9 nuclease is a SpyFi™ Cas9 nuclease.

54.-59. (canceled)

60. A population of modified hematopoietic stem and progenitor cells (HSPCs), wherein the HSPCs comprise an insertion and/or deletion in an endogenous gene locus encoding for CD33, wherein the insertion and/or deletion is mediated by a CD33-targeting guide RNA comprising:
a crRNA portion comprising (i) a guide sequence comprising the nucleic acid sequence GUCAGUGACGGUACAGGA (SEQ ID NO:1) or GAGUCAGUGACGGUACAGGA (SEQ ID NO:16), capable of hybridizing to a target endogenous gene locus encoding for CD33, and (ii) a repeat sequence; and
a tracrRNA portion comprising an anti-repeat sequence that is complementary to the repeat sequence.

61.-64. (canceled)

65. The population of modified hematopoietic stem and progenitor cells (HSPCs) of claim 60, wherein the guide RNA is a chemically modified CD33-targeting guide RNA
wherein the crRNA portion and/or the tracrRNA portion comprises one or more modified nucleotides, and wherein the insertion and/or deletion is capable of downregulating gene expression of CD33.

66. The population of modified hematopoietic stem and progenitor cells (HSPCs) of claim 65, further comprising wherein the insertion and/or deletion is mediated by
a single-stranded oligonucleotide donor (ssODN) sequence, wherein the ssODN sequence comprises the nucleotide sequence
(SEQ ID NO: 3)
GCAGGAGTCAGTGACGGTACAAGGAGGGTTTGTGCGTCC.

67.-68. (canceled)

69. The population of HSPCs of claim 66, wherein the ssODN sequence comprises the nucleotide sequence
(SEQ ID NO: 4)
GGTTGTCGGGCTGGGCCGAGCTGACCCTCGTTTCCCCACAGGGGCCCTGG
CTATGGATCCAAATTTCTGGCTGCAAGTGCAGGAGTCAGTGACGGTACAA
GGAGGGTTTGTGCGTCCTCGTGCCCTGCACTTTCTTCCATCCCATACCCT
ACTACGACAAGAACTCCCCAGTTCATGGTTACTGGTTCCGGGAAGGAGC.

70. The population of modified HSPCs of claim 65, wherein
(a) the population comprises at least about 20%, 25%, 30%, 35%, 40%, 45%, 50%, 55%, 60%, 65%, 70%, 75%, 80%, 85%, 90%, or 95% modified HSPCs comprising the insertion and/or deletion in the endogenous gene locus encoding for CD33, and/or
(b) the guide RNA is in a complex with a Cas9 nuclease, and/or
(c) the Cas9 nuclease is an S. pyogenes Cas9 nuclease or an S. aureus Cas9 nuclease, and/or
(d) the Cas9 nuclease is a variant S. pyogenes Cas9 nuclease, and/or
(e) the variant Cas9 nuclease comprises reduced off-target activity, and maintained or increased on-target activity relative to a wild-type Cas9, and/or
(f) the variant Cas9 nuclease is a SpyFi™ Cas9 nuclease.

71.-76. (canceled)

77. A method of treating a cancer in a subject in need thereof, the method comprising:
administering to the subject a CD33-targeted cell therapy comprising a CD33 CAR-T cell; and
administering to the subject a population of modified hematopoietic stem and progenitor cells (HSPCs) that are resistant to the CD33-targeted therapy,
wherein the HSPCs comprise an insertion and/or deletion in an endogenous gene locus encoding for CD33, wherein the insertion and/or deletion is mediated by a CD33− targeting guide RNA comprising:
a crRNA portion comprising (i) a guide sequence consisting of the nucleic acid sequence GUCAGUGACGGUACAGGA (SEQ ID NO:1) or GAGUCAGUGACGGUACAGGA (SEQ ID NO:16), capable of hybridizing to a target endogenous gene locus encoding for CD33, and (ii) a repeat sequence; and
a tracrRNA portion comprising an anti-repeat sequence that is complementary to the repeat sequence, and
wherein the insertion and/or deletion is capable of downregulating gene expression of CD33, thereby treating cancer in the subject.

78. The method of claim 77
further comprising wherein the insertion and/or deletion is mediated by
a single-stranded oligonucleotide donor (ssODN) sequence, wherein the ssODN sequence comprises the nucleotide sequence
(SEQ ID NO: 3)
GCAGGAGTCAGTGACGGTACAAGGAGGGTTTGTGCGTCC-,

79.-80. (canceled)

81. The method of claim 78, wherein the ssODN sequence comprises the nucleotide sequence
(SEQ ID NO: 4)
GGTTGTCGGGCTGGGCCGAGCTGACCCTCGTTTCCCCACAGGGGCCCTGG
CTATGGATCCAAATTTCTGGCTGCAAGTGCAGGAGTCAGTGACGGTACAA
GGAGGGTTTGTGCGTCCTCGTGCCCTGCACTTTCTTCCATCCCATACCCT
ACTACGACAAGAACTCCCCAGTTCATGGTTACTGGTTCCGGGAAGGAGC.

82. The method of claim 77,
wherein the guide RNA is a chemically modified CD33-targeting guide RNA,
wherein the crRNA portion and/or the tracrRNA portion comprises one or more modified nucleotides.

83.-86. (canceled)

87. The method of claim 77, wherein
(a) the HSPCs are administered to the subject prior to the CD33-targeted therapy, and/or
(b) the HSPCs are autologous cells obtained from a source selected from the group consisting of peripheral blood mononuclear cells, cord blood cells, bone marrow, lymph node, and spleen, and/or
(c) the HSPCs are CD34+ HSPCs, and/or
(d) the cancer is acute myeloid leukemia (AML), and/or
(e) the population of modified HSPCs comprise at least about 20%, 25%, 30%, 35%, 40%, 45%, 50%, 55%, 60%, 65%, 70%, 75%, 80%, 85%, 90%, or 95% modified HSPCs comprising the insertion and/or deletion capable of downregulating gene expression of CD33, and/or
(f) the guide RNA is in a complex with a Cas9 nuclease, and/or
(g) the Cas9 nuclease is an S. pyogenes Cas9 nuclease or an S. aureus Cas9 nuclease, and/or
(h) the Cas9 nuclease is a variant S. pyogenes Cas9 nuclease, and/or
(i) the variant Cas9 nuclease comprises reduced off-target activity, and maintained or increased on-target activity relative to a wild-type Cas9.

88.-95. (canceled)

96. A gene editing system comprising:
a) a chemically modified CD33-targeting guide RNA comprising a crRNA portion and a tracrRNA portion, wherein:
i) the crRNA portion comprises a guide sequence capable of hybridizing to a target endogenous gene locus encoding for CD33, and a repeat sequence; and
ii) the tracrRNA portion comprises an anti-repeat sequence that is complementary to the repeat sequence,
wherein the crRNA portion and/or the tracrRNA portion comprise(s) one or more modified nucleotides; and
b) an RNA-guided nuclease.

97. The gene editing system of claim 96, wherein
(a) the RNA-guided nuclease is a Cas9 nuclease, and/or
(b) the Cas9 nuclease is an S. pyogenes Cas9 nuclease or an S. aureus Cas9 nuclease, and/or
(c) the Cas9 nuclease is a variant S. pyogenes Cas9 nuclease, and/or
(d) the variant Cas9 nuclease comprises reduced off-target activity and maintained or increased on-target activity relative to a wild-type Cas9 nuclease, and/or
(e) the variant Cas9 nuclease is a SpyFi™ Cas9 nuclease.

98.-101. (canceled)


SEQUENCE LISTING (18 sequences)

SEQ ID NO: 1 - RNA, 18 nt, Artificial Sequence
  guide sequence
gucagugacgguacagga

SEQ ID NO: 2 - RNA, 18 nt, Artificial Sequence
  guide sequence
  modified_base (1)..(3): 2′-O-methyl modification and phosphorothioate modification
gucagugacgguacagga

SEQ ID NO: 3 - DNA, 39 bp, Artificial Sequence
  ssODN
gcaggagtcagtgacggtacaaggagggtttgtgcgtcc

SEQ ID NO: 4 - DNA, 199 bp, Artificial Sequence
  ssODN
ggttgtcgggctgggccgagctgaccctcgtttccccacaggggccctggctatggatccaaatttctggctgcaagtgcaggagtcagtgacggtacaaggagggtttgtgcgtcctcgtgccctgcactttcttccatcccataccctactacgacaagaactccccagttcatggttactggttccgggaaggagc

SEQ ID NO: 5 - DNA, 20 bp, Artificial Sequence
  guide sequence
tggggtgattatgagcaccg

SEQ ID NO: 6 - DNA, 17 bp, Artificial Sequence
  guide sequence
ggtgattatgagcaccg

SEQ ID NO: 7 - DNA, 20 bp, Artificial Sequence
  guide sequence
tgagcatcgtagacgccagg

SEQ ID NO: 8 - DNA, 17 bp, Artificial Sequence
  guide sequence
gcatcgtagacgccagg

SEQ ID NO: 9 - DNA, 20 bp, Artificial Sequence
  guide sequence
atccctggcactctagaacc

SEQ ID NO: 10 - DNA, 18 bp, Artificial Sequence
  guide sequence
gcctggcactctagaacc

SEQ ID NO: 11 - DNA, 20 bp, Artificial Sequence
  guide sequence
gagtcagtgacggtacagga

SEQ ID NO: 12 - DNA, 18 bp, Artificial Sequence
  guide sequence
gtcagtgacggtacagga

SEQ ID NO: 13 - DNA, 20 bp, Artificial Sequence
  guide sequence
tgtcacatgcacagagagct

SEQ ID NO: 14 - DNA, 18 bp, Artificial Sequence
  guide sequence
gcacatgcacagagagct

SEQ ID NO: 15 - DNA, 20 bp, Artificial Sequence
  guide sequence
gagtccgagcagaagaagaa

SEQ ID NO: 16 - RNA, 20 nt, Artificial Sequence
  guide sequence
gagucagugacgguacagga

SEQ ID NO: 17 - RNA, 20 nt, Artificial Sequence
  guide sequence
  modified_base (1)..(3): 2′-O-methyl modification and phosphorothioate modification
gagucagugacgguacagga

SEQ ID NO: 18 - DNA, 38 bp, Artificial Sequence
  ssODN
gcaggagtcagtgacggtacaggagggtttgtgcgtcc